(19) United States
(12) Patent Application Publication
(10) Pub. No.: US 2023/0310441 A1
(43) Pub. Date: Oct. 5, 2023

(54) TREATMENT FOR SCHIZOPHRENIA

(71) Applicant: TAKEDA PHARMACEUTICAL COMPANY, Osaka-shi, Osaka (JP)

(72) Inventors: Dimitrios ARKILO, Cambridge, MA (US); Joy ATIENZA, Lexington, MA (US); Antonio LAURENZA, Cambridge, MA (US); Hans SCHIFFER, San Diego, CA (US); Johannes TAUSCHER, Cambridge, MA (US); Wei YIN, Cambrodge, MA (US)

(21) Appl. No.: 18/245,518

(22) PCT Filed: Sep. 17, 2021
(86) PCT No.: PCT/IB2021/000628

§ 371 Date: Mar. 15, 2023

Related U.S. Application Data
(60) Provisional application No. 63/081,264, filed on Sep. 21, 2020.

(51) Int. Cl. A61K 31/53; A61K 45/06; A61P 25/18; A61K 9/00; A61K 9/28; A61K 9/20

(52) U.S. Cl. CPC A61K 31/53; A61K 45/06; A61P 25/18; A61K 9/0053; A61K 9/2866; A61K 9/2853; A61K 9/2813; A61K 9/2054; A61K 9/2018; A61K 9/2013

(57) ABSTRACT

Methods of treating schizophrenia comprising administering at least one compound chosen from Compound (I):

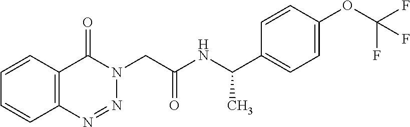 [SMILES: C[C@H](NC(=O)Cn1nnc2ccccc2c1=O)c1ccc(OC(F)(F)F)cc1]

and pharmaceutically acceptable salts thereof are disclosed. Pharmaceutical compositions comprising at least one compound chosen from Compound (I) and pharmaceutically acceptable salts thereof are also disclosed.

This application claims the benefit of priority of U.S. Provisional Application No. 63/081,264, filed Sep. 21, 2020, the contents of which are incorporated by reference herein in their entirety.

Disclosed herein are methods of treating schizophrenia. Modulators of G-protein-coupled receptor 139 (GPR139) and pharmaceutical compositions comprising the modulators are also disclosed.

Schizophrenia is a severe mental disorder that affects approximately 1% of the population, with lifetime prevalence estimates ranging from 5.6 to 11.9 per 1000 persons (Sahu et al., 2016; Coyle, 2017; McGrath et al., 2008). Schizophrenia is characterized by psychosis, cognitive impairments, and social and motivational deficits. For example, schizophrenia may be characterized by positive symptoms (e.g., hallucinations or delusions), negative symptoms (e.g., anhedonia, avolition, blunted affect, reduced spontaneous speech, and social withdrawal), and cognitive impairment associated with schizophrenia (Owen et al., 2016). Cognitive symptoms of schizophrenia affect a wide range of domains, including, but not limited to, attention, working memory, and executive functions. While positive symptoms of schizophrenia tend to relapse and remit, negative and cognitive symptoms of schizophrenia are often chronic and impact social functioning for those afflicted.

The dopamine D2 receptor is the primary direct therapeutic target of current medications for schizophrenia. While these therapies are generally efficacious for treating positive symptoms of schizophrenia, many patients fail to respond adequately (Lally et al., 2016). In addition, treatments targeting the dopamine D2 receptor appear to have little significant clinical impact on negative symptoms of schizophrenia (such as, e.g., anhedonia, loss of motivation, and reduced interest in social interaction) and unclear efficacy in treating the cognitive deficits frequently present in schizophrenia patients (Fusar-Poli et al., 2015; Sakurai et al., 2013).

Although negative and cognitive symptoms are highly predictive of quality of life and functional recovery (Hunter and Barry, 2012), there are no approved treatments for the cognitive impairment associated with schizophrenia. Accordingly, there is a need for novel treatments of schizophrenia.

GPR139 is a highly conserved, class A orphan G-protein coupled receptor belonging to the gamma rhodopsin family. GPR139 may be coupled with Gs, Gq and Gi signaling and appears to be constitutively active when recombinantly expressed in mammalian cells. GPR139 is abundantly expressed in the central nervous system (CNS), to a lesser extent in the pancreas and pituitary, and at low levels in other peripheral tissue. GPR139 expression is particularly high in the habenula, along with the striatum, hypothalamus, and midbrain regions (Vedel et al., 2019).

The habenula is a highly conserved subcortical structure that plays an important role in reward and cognition networks (Bianco and Wilson, 2009). The habenula receives inputs from the basal ganglia and limbic system and sends outputs to midbrain and forebrain structures containing dopaminergic and serotonergic neurons. The habenula has been shown to be involved in regulating downstream monoamine neurotransmitter activity (Liu et al., 2015), with habenula efferents modulating both dopamine (DA) and serotonin (5HT) cell groups in the midbrain (Wang and Aghajanian, 1977; Christoph et al., 1986; Jhou et al., 2009; Sego et al., 2014). Habenular nuclei are involved in pain processing, reproductive behavior, nutrition, sleep-wake cycles, stress responses, and learning.

Several studies have suggested involvement of the habenula in schizophrenia, with multiple reports in humans and animals linking aberrant activity in the habenula with schizophrenia, often within the negative or cognitive symptom domains. Chronic treatment with cocaine or amphetamine damages the output pathways of the habenula in rats, resulting in a schizophrenic-like state. Large calcifications in the pineal and habenula are more common in people suffering from schizophrenia than normal controls. Additionally, altered activation of the habenula in patients with schizophrenia has been demonstrated in an fMRI study. Moreover, following an error in a difficult matching-to-sample task, the habenula was activated in control subjects, but not in patients with schizophrenia.

Human and animal genetic data have implicated GPR139 in schizophrenia. For example, in a study of 6 pairs of monozygotic human twins discordant for schizophrenia, copy number variants of GPR139 were reported in the affected twin in one of the pairs (Castellani et al., 2014). Additionally, GPR139−/− knockout mice were found to be comparable to wild-type on a range of standard tasks but significantly impaired on tasks related to negative symptomatology (such as, e.g., progressive ratio (motivation) and nest building (self-neglect)) and in a novel object recognition model of working memory (Atienza et al., 2018). Thus, modulators of GPR139 (such as, e.g., GPR139 agonists) may be useful for treating schizophrenia, including treating negative and cognitive symptoms of schizophrenia.

Compound (I) is a GPR139 agonist of the following structure:

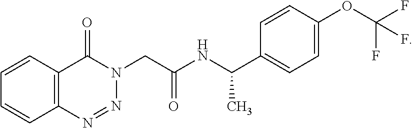 [SMILES: C[C@H](NC(=O)Cn1nnc2ccccc2c1=O)c1ccc(OC(F)(F)F)cc1]

See PCT Publication No. WO 2016/081736, which is incorporated herein by reference, e.g., Example 2.

Disclosed herein are methods of treating schizophrenia comprising administering to a patient in need thereof more than 80 mg of at least one compound chosen from Compound (I) and pharmaceutically acceptable salts thereof.

In some embodiments, more than 100 mg of at least one compound chosen from Compound (I) and pharmaceutically acceptable salts thereof is administered. In some embodiments, 120 mg of at least one compound chosen from Compound (I) and pharmaceutically acceptable salts thereof is administered. In some embodiments, 160 mg of at least one compound chosen from Compound (I) and pharmaceutically acceptable salts thereof is administered. In some embodiments, 160 mg of at least one compound chosen from Compound (I) and pharmaceutically acceptable salts thereof is administered in the form of 4 tablets, each tablet comprising 40 mg of the at least one compound chosen from Compound (I) and pharmaceutically acceptable salts thereof.

In some embodiments, the at least one compound chosen from Compound (I) and pharmaceutically acceptable salts thereof is effective to achieve a mean Cₘₐₓ in the patient of at least 500 ng/mL. In some embodiments, the at least one compound chosen from Compound (I) and pharmaceutically acceptable salts thereof is effective to achieve a mean Cₘₐₓ in the patient of not more than 2500 ng/mL.

In some embodiments, the at least one compound chosen from Compound (I) and pharmaceutically acceptable salts thereof is effective to achieve a mean Cₘₐₓ in the patient from 500 ng/mL to 2500 ng/mL.

In some embodiments, the at least one compound chosen from Compound (I) and pharmaceutically acceptable salts thereof is effective to achieve a mean Cₘₐₓ in the patient from 50% to 150% of 1267 ng/mL.

In some embodiments, the at least one compound chosen from Compound (I) and pharmaceutically acceptable salts thereof is orally administered. In some embodiments, the at least one compound chosen from Compound (I) and pharmaceutically acceptable salts thereof is administered in at least one tablet. In some embodiments, the at least one compound chosen from Compound (I) and pharmaceutically acceptable salts thereof is administered in more than one tablet (e.g., 2, 3, or 4 tablets). In some embodiments, the at least one compound chosen from Compound (I) and pharmaceutically acceptable salts thereof is administered in an oral suspension.

In some embodiments, the at least one compound chosen from Compound (I) and pharmaceutically acceptable salts thereof is administered with water.

In some embodiments, the at least one compound chosen from Compound (I) and pharmaceutically acceptable salts thereof is administered without food. In some embodiments, the at least one compound chosen from Compound (I) and pharmaceutically acceptable salts thereof is administered with food. In some embodiments, the at least one compound chosen from Compound (I) and pharmaceutically acceptable salts thereof is administered with a high-fat, high-calorie meal.

In some embodiments, the methods further comprise administering at least one additional active pharmaceutical ingredient. In some embodiments, the at least one additional active pharmaceutical ingredient is chosen from sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, mGlu2/3 agonists, 5HT-2 antagonists, PDE10 antagonists, and GlyT1 inhibitors. In some embodiments, the at least one additional active pharmaceutical ingredient is chosen from antipsychotics.

Also provided herein are methods of treating schizophrenia comprising administering to a patient in need thereof: a loading dose comprising more than 20 mg of at least one compound chosen from Compound (I) and pharmaceutically acceptable salts thereof; and at least one weekly maintenance dose comprising at least one compound chosen from Compound (I) and pharmaceutically acceptable salts thereof.

In some embodiments, the at least one weekly maintenance dose is administered 5 to 9 days after the loading dose. In some embodiments, the at least one weekly maintenance dose is administered 7 days after the loading dose.

In some embodiments, the loading dose comprises 40 mg of at least one compound chosen from Compound (I) and pharmaceutically acceptable salts thereof. In some embodiments, the loading dose comprises 80 mg of at least one compound chosen from Compound (I) and pharmaceutically acceptable salts thereof. In some embodiments, the loading dose comprises 120 mg of at least one compound chosen from Compound (I) and pharmaceutically acceptable salts thereof. In some embodiments, the loading dose comprises 160 mg of at least one compound chosen from Compound (I) and pharmaceutically acceptable salts thereof. In some embodiments, the loading dose is administered in the form of 4 tablets, each tablet comprising 40 mg of the at least one compound chosen from Compound (I) and pharmaceutically acceptable salts thereof.

In some embodiments, the loading dose is effective to achieve a mean Cₘₐₓ in the patient of at least 250 ng/mL. In some embodiments, the loading dose is effective to achieve a mean Cₘₐₓ in the patient of not more than 2500 ng/mL. In some embodiments, the loading dose is effective to achieve a mean Cₘₐₓ in the patient from 500 ng/mL to 2500 ng/mL. In some embodiments, the loading dose is effective to achieve a mean Cₘₐₓ in the patient from 50% to 150% of 1267 ng/mL. In some embodiments, the loading dose is effective to achieve a mean Cₘₐₓ in the patient from 75% to 125% of 1267 ng/mL.

In some embodiments, the at least one weekly maintenance dose is half the loading dose.

In some embodiments, the at least one weekly maintenance dose comprises 20 mg of at least one compound chosen from Compound (I) and pharmaceutically acceptable salts thereof. In some embodiments, the at least one weekly maintenance dose comprises 40 mg of at least one compound chosen from Compound (I) and pharmaceutically acceptable salts thereof. In some embodiments, the at least one weekly maintenance dose comprises 60 mg of at least one compound chosen from Compound (I) and pharmaceutically acceptable salts thereof. In some embodiments, the at least one weekly maintenance dose comprises 80 mg of at least one compound chosen from Compound (I) and pharmaceutically acceptable salts thereof. In some embodiments, the at least one weekly maintenance dose is administered in the form of 2 tablets each comprising at least one compound chosen from Compound (I) and pharmaceutically acceptable salts thereof.

In some embodiments, the at least one weekly maintenance dose is effective to achieve a mean Cₘₐₓ at steady state in the patient of at least 400 ng/mL. In some embodiments, the at least one weekly maintenance dose is effective to achieve a mean Cₘₐₓ at steady state in the patient of not more than 3000 ng/mL. In some embodiments, the at least one weekly maintenance dose is effective to achieve a mean Cₘₐₓ at steady state in the patient from 400 ng/mL to 3000 ng/mL. In some embodiments, the at least one weekly maintenance dose is effective to achieve a mean Cₘₐₓ at steady state in the patient from 50% to 150% of 1885 ng/mL. In some embodiments, the loading dose is effective to achieve a mean Cₘₐₓ in the patient from 75% to 125% of 1885 ng/mL.

In some embodiments, the at least one weekly maintenance dose is effective to achieve a mean C_{av,ss} in the patient of at least 200 ng/mL. In some embodiments, the at least one weekly maintenance dose is effective to achieve a mean C_{av,ss} in the patient of not more than 2500 ng/mL. In some embodiments, the at least one weekly maintenance dose is effective to achieve a mean C_{av,ss} in the patient from 200 ng/mL to 2500 ng/mL. In some embodiments, the at least one weekly maintenance dose is effective to achieve a mean C_{av,ss} in the patient from 50% to 150% of 1353 ng/mL. In some embodiments, the at least one weekly maintenance dose is effective to achieve a mean C_{av,ss} in the patient from 75% to 125% of 1353 ng/mL.

In some embodiments, the mean AUC_{τ} at steady state in the patient is not more than 400,000 ng·h/mL. In some embodiments, the mean AUC_{τ} at steady state in the patient is not more than 300,000 ng·h/mL. In some embodiments, the mean AUC_{τ} at steady state in the patient is from 30,000 ng·h/mL to 400,000 ng·h/mL. In some embodiments, the mean AUC_{τ} at steady state in the patient is from 50% to 150% of 227,230 ng·h/mL. In some embodiments, the mean AUC_{τ} at steady state in the patient is from 75% to 125% of 227,230 ng·h/mL.

In some embodiments, the loading dose comprises 40 mg of at least one compound chosen from Compound (I) and pharmaceutically acceptable salts thereof, and the at least one weekly maintenance dose comprises 20 mg of at least one compound chosen from Compound (I) and pharmaceutically acceptable salts thereof. In some embodiments, the loading dose comprises 80 mg of at least one compound chosen from Compound (I) and pharmaceutically acceptable salts thereof, and the at least one weekly maintenance dose comprises 40 mg of at least one compound chosen from Compound (I) and pharmaceutically acceptable salts thereof. In some embodiments, the loading dose comprises 120 mg of at least one compound chosen from Compound (I) and pharmaceutically acceptable salts thereof, and the at least one weekly maintenance dose comprises 60 mg of at least one compound chosen from Compound (I) and pharmaceutically acceptable salts thereof. In some embodiments, the loading dose comprises 160 mg of at least one compound chosen from Compound (I) and pharmaceutically acceptable salts thereof, and the at least one weekly maintenance dose comprises 80 mg of at least one compound chosen from Compound (I) and pharmaceutically acceptable salts thereof. In some embodiments, the loading dose is administered in the form of 4 tablets each comprising 40 mg of at least one compound chosen from Compound (I) and pharmaceutically acceptable salts thereof, and the at least one weekly maintenance dose is administered in the form of 2 tablets each comprising 40 mg of at least one compound chosen from Compound (I) and pharmaceutically acceptable salts thereof.

In some embodiments, the loading dose is orally administered. In some embodiments, the loading dose is administered in at least one tablet. In some embodiments, the loading dose is administered in more than one tablet (e.g., 2, 3, or 4 tablets). In some embodiments, the loading dose is administered in an oral suspension.

In some embodiments, the loading dose is administered with water. In some embodiments, the loading dose is administered with food. In some embodiments, the loading dose is administered without food. In some embodiments, the loading dose is administered with food. In some embodiments, the loading dose is administered with a high-fat, high-calorie meal.

In some embodiments, the at least one weekly maintenance dose is orally administered. In some embodiments, the at least one weekly maintenance dose is administered in at least one tablet. In some embodiments, the at least one weekly maintenance dose is administered in more than one tablet (e.g., 2, 3, or 4 tablets). In some embodiments, the at least one weekly maintenance dose is administered in an oral suspension.

In some embodiments, the at least one weekly maintenance dose is administered with water. In some embodiments, the at least one weekly maintenance dose is administered without food. In some embodiments, the at least one weekly maintenance dose is administered with food. In some embodiments, the at least one weekly maintenance dose is administered with a high-fat, high-calorie meal.

In some embodiments, the methods further comprise administering at least one additional active pharmaceutical ingredient. In some embodiments, the at least one additional active pharmaceutical ingredient is chosen from sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, mGlu2/3 agonists, 5HT-2 antagonists, PDE10 antagonists, and GlyT1 inhibitors. In some embodiments, the at least one additional active pharmaceutical ingredient is chosen from antipsychotics.

Also disclosed herein are methods of treating negative symptoms of schizophrenia comprising administering at least one compound chosen from Compound (I) and pharmaceutically acceptable salts thereof to a patient in need thereof. In some embodiments, methods of this disclosure treat at least one negative symptom of schizophrenia chosen from anhedonia, loss of motivation, and reduced interest in social interaction.

Also disclosed herein are methods of treating cognitive symptoms of schizophrenia comprising administering at least one compound chosen from Compound (I) and pharmaceutically acceptable salts thereof to a patient in need thereof. In some embodiments, methods of this disclosure treat at least one cognitive symptom of schizophrenia chosen from impaired verbal memory, impaired working memory, impaired motor function, impaired attention and processing speed, impaired verbal fluency, and impaired executive function. In some embodiments, methods of this disclosure modulate reward anticipation related brain activity in the patient. In some embodiments, methods of this disclosure modulate cerebral blood flow in the patient. In some embodiments, methods of this disclosure increase ventral striatal activity during reward anticipation in the patient.

Also disclosed herein are methods of modulating dopamine release in a patient with schizophrenia comprising administering at least one compound chosen from Compound (I) and pharmaceutically acceptable salts thereof to the patient. In some embodiments, methods of this disclosure reduce dopamine release in the patient following an exposure to a stimulant.

Also disclosed herein are pharmaceutical compositions comprising: at least one compound chosen from Compound (I) and pharmaceutically acceptable salts thereof; and at least one pharmaceutically acceptable excipient. In some embodiments, the pharmaceutical compositions comprise more than 80 mg of at least one compound chosen from Compound (I) and pharmaceutically acceptable salts thereof. In some embodiments, the pharmaceutical compositions comprise more than 100 mg of at least one compound chosen from Compound (I) and pharmaceutically acceptable salts thereof. In some embodiments, the pharmaceutical compositions comprise 120 mg of at least one compound chosen from Compound (I) and pharmaceutically acceptable salts thereof. In some embodiments, the pharmaceutical compositions comprise 160 mg of at least one compound chosen from Compound (I) and pharmaceutically acceptable salts thereof.

In some embodiments, the pharmaceutical composition is in the form of at least one tablet. In some embodiments, the pharmaceutical composition is in the form of more than one tablet (e.g., 2, 3, or 4 tablets). In some embodiments, the pharmaceutical composition is in the form of at least one immediate release tablet.

In some embodiments, the pharmaceutical composition is in the form of an oral suspension.

### BRIEF DESCRIPTION OF THE DRAWINGS

FIG. 1 depicts plasma concentration-time curves of Compound (I) following multiple oral administration of Compound (I) (Day 22) in healthy participants and patients with stable schizophrenia (A) 0-22 h post-dose and (B) full profile. Healthy participants and patients with stable schizophrenia were administered 160 mg Compound (I) on Day 1, followed by once weekly 80 mg Compound (I) oral administration. Zero was used for below the limit of quantification values (<1.00 ng/mL).

FIG. 2 depicts the study design for a [¹¹C]PHNO PET investigation of endogenous dopamine release in the brains of healthy human volunteers following the administration of Compound (I) (20 mg or 40 mg).

FIG. 3 depicts within-subject differences between Compound (I) and placebo in the average activation of the ventral striatum during a Monetary Incentive Delay (MID) reward task (mean±standard deviation (SD) by visit) in patients with stable schizophrenia. A checkmark indicates that the statistical success criterion (the posterior probability that the increase over placebo is greater than 0.09 were at least 70%) was met.

FIG. 4 depicts global cerebral blood flow (CBF) (mean±standard error (SE)) in patients with stable schizophrenia treated with Compound (I) (40 mg or 160 mg).

FIG. 5 depicts a manufacturing flow diagram for tablets comprising at least one compound chosen from Compound (I) and pharmaceutically acceptable salts thereof.

### DEFINITIONS

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “active pharmaceutical ingredient” or “therapeutic agent” (“API”) refers to a biologically active compound.

As used herein, “administration” of an API to a patient refers to any route (e.g., oral delivery) of introducing or delivering the API to the patient. Administration includes self-administration and the administration by another.

As used herein, the term “agonist” refers to a molecule (e.g., an API) that binds to a receptor (e.g., GPR139) and activates the receptor. As used herein, “agonist” refers to both full agonists and partial agonists.

As used herein, the term “anhedonia” refers to one or more deficits in hedonic function, such as, e.g., reduced motivation or ability to experience pleasure.

As used herein, the term “avolition” refers to a decrease in motivation to initiate and perform goal-directed behaviors.

As used herein, “Compound (I)” refers to (S)-2-(4-oxobenzo[d][1,2,3]triazin-3(4H)-yl)-N-(1-(4-(trifluoromethoxy)phenyl)ethyl)acetamide, which has the following structure:

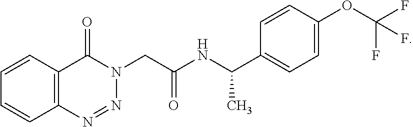 [SMILES: C[C@H](NC(=O)Cn1nnc2ccccc2c1=O)c1ccc(OC(F)(F)F)cc1]

Herein, Compound (I) may be referred to as a “drug,” “active agent,” “a therapeutic agent,” or an “API.”

As used herein, a “condition,” “disorder,” or “disease” relates to any unhealthy or abnormal state.

As used herein, an “effective amount” or “effective dose” refers to an amount of a molecule that treats, upon single or multiple dose administration, a patient suffering from a condition. An effective amount can be determined by the attending diagnostician through the use of known techniques and by observing results obtained under analogous circumstances. In determining the effective amount, a number of factors are considered by the attending diagnostician, including, but not limited to: the species of patient; its size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances.

As used herein, an amount expressed in terms of “mg of at least one compound chosen from Compound (I) and pharmaceutically acceptable salts thereof” refers to the total amount in milligrams of Compound (I), i.e., the free base, plus the equivalent amount of one or more pharmaceutically acceptable salts of Compound (I) based on the weight of free base therein.

As used herein, the term “increase” refers to altering positively by at least 5%, including, but not limited to, altering positively by 5%, altering positively by 10%, altering positively by 25%, altering positively by 30%, altering positively by 50%, altering positively by 75%, or altering positively by 100%.

As used herein, “mammals” refer to domesticated animals (e.g., dogs, cats, and horses) and humans. In some embodiments, the mammal is a human.

As used herein, the phrase “mean [X] in the patient” refers to the mean value of [X] determined from one or more observations of [X] in the patient.

As used herein, the term “modulate” refers to altering positively or negatively. Non-limiting example modulations include a 1% change, a 2% change, a 5% change, a 10% change, a 25% change, a 50% change, a 75% change, or a 100% change.

As used herein, the terms “patient” and “subject” are used interchangeably and refer to a mammal, such as, e.g., a human.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use. As a non-limiting example, pharmaceutically acceptable excipients may be solid, semi-solid, or liquid materials which in the aggregate can serve as a vehicle or medium for the active ingredient. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

As used herein, the term “pharmaceutically acceptable salt” refers to a non-toxic salt form of a compound of this disclosure. Pharmaceutically acceptable salts of Compound (I) of this disclosure include those derived from suitable inorganic and organic acids and bases. Pharmaceutically acceptable salts are well known in the art. Suitable pharmaceutically acceptable salts are, e.g., those disclosed in Berge, S. M., et al. J. Pharma. Sci. 66:1-19 (1977). Non-limiting examples of pharmaceutically acceptable salts disclosed in that article include: acetate; benzenesulfonate; benzoate; bicarbonate; bitartrate; bromide; calcium edetate; camsylate; carbonate; chloride; citrate; dihydrochloride; edetate; edisylate; estolate; esylate; fumarate; gluceptate; gluconate; glutamate; glycollylarsanilate; hexylresorcinate; hydrabamine; hydrobromide; hydrochloride; hydroxynaphthoate; iodide; isethionate; lactate; lactobionate; malate; maleate; mandelate; mesylate; methylbromide; methylnitrate; methylsulfate; mucate; napsylate; nitrate; pamoate (embonate); pantothenate; phosphate/diphosphate; polygalacturonate; salicylate; stearate; subacetate; succinate; sulfate; tannate; tartrate; teociate; triethiodide; benzathine; chloroprocaine; choline; diethanolamine; ethylenediamine; meglumine; procaine; aluminum; calcium; lithium; magnesium; potassium; sodium; and zinc.

Non-limiting examples of pharmaceutically acceptable salts derived from appropriate acids include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Additional non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Non-limiting examples of pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other non-limiting examples of pharmaceutically acceptable salts include besylate and glucosamine salts.

As used herein, the term “reduce” refers to altering negatively by at least 5% including, but not limited to, altering negatively by 5%, altering negatively by 10%, altering negatively by 25%, altering negatively by 30%, altering negatively by 50%, altering negatively by 75%, or altering negatively by 100%.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

As used herein, the term “weekly” means per week. In some embodiments, “weekly” means every 7 days±2 days.

### EXAMPLE EMBODIMENTS

Without limitation, some embodiments of the disclosure include:
1. A method of treating schizophrenia comprising administering to a patient in need thereof more than 80 mg of at least one compound chosen from Compound (I):

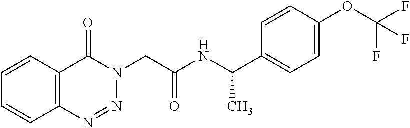 [SMILES: C[C@H](NC(=O)Cn1nnc2ccccc2c1=O)c1ccc(OC(F)(F)F)cc1]

and pharmaceutically acceptable salts thereof.
2. The method of embodiment 1, wherein more than 100 mg of at least one compound chosen from Compound (I) and pharmaceutically acceptable salts thereof is administered.
3. The method of embodiment 1 or 2, wherein 120 mg of at least one compound chosen from Compound (I) and pharmaceutically acceptable salts thereof is administered.
4. The method of embodiment 1 or 2, wherein 160 mg of at least one compound chosen from Compound (I) and pharmaceutically acceptable salts thereof is administered.
5. The method of any one of embodiments 1 to 4, wherein the at least one compound chosen from Compound (I) and pharmaceutically acceptable salts thereof is effective to achieve a mean Cₘₐₓ in the patient of at least 500 ng/mL.
6. The method of any one of embodiments 1 to 5, wherein the at least one compound chosen from Compound (I) and pharmaceutically acceptable salts thereof is effective to achieve a mean Cₘₐₓ in the patient of not more than 2500 ng/mL.
7. The method of any one of embodiments 1 to 6, wherein the at least one compound chosen from Compound (I) and pharmaceutically acceptable salts thereof is effective to achieve a mean Cₘₐₓ in the patient from 500 ng/mL to 2500 ng/mL.
8. The method of embodiment 4, wherein the at least one compound chosen from Compound (I) and pharmaceutically acceptable salts thereof is effective to achieve a mean Cₘₐₓ in the patient from 50% to 150% of 1267 ng/mL.
9. The method of any one of embodiments 1 to 8, wherein the at least one compound chosen from Compound (I) and pharmaceutically acceptable salts thereof is orally administered.
10. The method of any one of embodiments 1 to 9, wherein the at least one compound chosen from Compound (I) and pharmaceutically acceptable salts thereof is administered in at least one tablet.
11. The method of any one of embodiments 1 to 9, wherein the at least one compound chosen from Compound (I) and pharmaceutically acceptable salts thereof is administered in an oral suspension.
12. The method of any one of embodiments 1 to 11, wherein the at least one compound chosen from Compound (I) and pharmaceutically acceptable salts thereof is administered without food.
13. The method of any one of embodiments 1 to 11, wherein the at least one compound chosen from Compound (I) and pharmaceutically acceptable salts thereof is administered with food.
14. The method of any one of embodiments 1 to 13, further comprising administering at least one additional active pharmaceutical ingredient.
15. The method of embodiment 14, wherein the at least one additional active pharmaceutical ingredient is chosen from antipsychotics.
16. The method of any one of embodiments 1 to 15, comprising treating at least one negative symptom of schizophrenia.
17. The method of embodiment 16, wherein the at least one negative symptom of schizophrenia is chosen from anhedonia, loss of motivation, and reduced interest in social interaction.
18. The method of any one of embodiments 1 to 17, comprising treating at least one cognitive symptom of schizophrenia.
19. The method of embodiment 18, wherein the at least one cognitive symptom of schizophrenia is chosen from impaired verbal memory, impaired working memory, impaired motor function, impaired attention and processing speed, impaired verbal fluency, and impaired executive function.
20. The method of any one of embodiments 1 to 19, comprising modulating reward anticipation related brain activity in the patient.
21. The method of any one of embodiments 1 to 20, comprising modulating cerebral blood flow in the patient.
22. The method of any one of embodiments 1 to 21, comprising increasing ventral striatal activity during reward anticipation in the patient.
23. The method of any one of embodiments 1 to 22, comprising modulating dopamine release in the patient.
24. A method of treating schizophrenia comprising administering to a patient in need thereof:
a loading dose comprising more than 20 mg of at least one compound chosen from Compound (I):

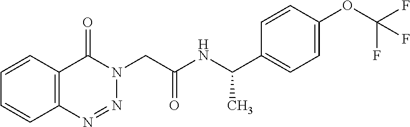 [SMILES: C[C@H](NC(=O)Cn1nnc2ccccc2c1=O)c1ccc(OC(F)(F)F)cc1]

and pharmaceutically acceptable salts thereof, and
at least one weekly maintenance dose comprising at least one compound chosen from Compound (I) and pharmaceutically acceptable salts thereof.
25. The method of embodiment 24, wherein the at least one weekly maintenance dose is administered 5 to 9 days after the loading dose.
26. The method of embodiment 24 or 25, wherein the at least one weekly maintenance dose is administered 7 days after the loading dose.
27. The method of any one of embodiments 24 to 26, wherein the loading dose comprises 40 mg of at least one compound chosen from Compound (I) and pharmaceutically acceptable salts thereof.
28. The method of any one of embodiments 24 to 26, wherein the loading dose comprises 80 mg of at least one compound chosen from Compound (I) and pharmaceutically acceptable salts thereof.
29. The method of any one of embodiments 24 to 26, wherein the loading dose comprises 120 mg of at least one compound chosen from Compound (I) and pharmaceutically acceptable salts thereof.
30. The method of any one of embodiments 24 to 26, wherein the loading dose comprises 160 mg of at least one compound chosen from Compound (I) and pharmaceutically acceptable salts thereof.
31. The method of any one of embodiments 24 to 30, wherein the loading dose is effective to achieve a mean Cₘₐₓ in the patient of at least 250 ng/mL.
32. The method of any one of embodiments 24 to 31, wherein the loading dose is effective to achieve a mean Cₘₐₓ in the patient of not more than 2500 ng/mL.
33. The method of any one of embodiments 24 to 32, wherein the loading dose is effective to achieve a mean Cₘₐₓ in the patient from 500 ng/mL to 2500 ng/mL.
34. The method of embodiment 30, wherein the loading dose is effective to achieve a mean Cₘₐₓ in the patient from 50% to 150% of 1267 ng/mL.
35. The method of any one of embodiments 24 to 34, wherein the at least one weekly maintenance dose is half the loading dose.
36. The method of embodiment 30 or 34, wherein the at least one weekly maintenance dose comprises 80 mg of at least one compound chosen from Compound (I) and pharmaceutically acceptable salts thereof.
37. The method of any one of embodiments 24 to 36, wherein the at least one weekly maintenance dose is effective to achieve a mean Cₘₐₓ at steady state in the patient of at least 400 ng/mL.
38. The method of any one of embodiments 24 to 37, wherein the at least one weekly maintenance dose is effective to achieve a mean Cₘₐₓ at steady state in the patient of not more than 3000 ng/mL.
39. The method of any one of embodiments 24 to 38, wherein the at least one weekly maintenance dose is effective to achieve a mean Cₘₐₓ at steady state in the patient from 400 ng/mL to 3000 ng/mL.
40. The method of embodiment 36, wherein the at least one weekly maintenance dose is effective to achieve a mean Cₘₐₓ at steady state in the patient from 50% to 150% of 1885 ng/mL.
41. The method of any one of embodiments 24 to 40, wherein the at least one weekly maintenance dose is effective to achieve a mean C_{av,ss} in the patient of at least 200 ng/mL.
42. The method of any one of embodiments 24 to 41, wherein the at least one weekly maintenance dose is effective to achieve a mean C_{av,ss} in the patient of not more than 2500 ng/mL.
43. The method of any one of embodiments 24 to 42, wherein the at least one weekly maintenance dose is effective to achieve a mean C_{av,ss} in the patient from 200 ng/mL to 2500 ng/mL.
44. The method of embodiment 36 or 40, wherein the at least one weekly maintenance dose is effective to achieve a mean C_{av,ss} in the patient from 50% to 150% of 1353 ng/mL.
45. The method of any one of embodiments 24 to 44, wherein the mean AUC_{τ} at steady state in the patient is not more than 400,000 ng·h/mL.
46. The method of any one of embodiments 24 to 45, the mean AUC_{τ} at steady state in the patient is not more than 300,000 ng·h/mL.
47. The method of any one of embodiments 24 to 46, wherein the mean AUC_{τ} at steady state in the patient is from 30,000 ng·h/mL to 400,000 ng·h/mL.
48. The method of any one of embodiments 36, 40, or 44, wherein the mean AUC_{τ} at steady state in the patient is from 50% to 150% of 227,230 ng·h/mL.
49. The method of any one of embodiments 24 to 48, wherein the loading dose is orally administered.
50. The method of any one of embodiments 24 to 49, wherein the loading dose is administered in at least one tablet.
51. The method of any one of embodiments 24 to 49, wherein the loading dose is administered in an oral suspension.
52. The method of any one of embodiments 24 to 51, wherein the loading dose is administered without food.
53. The method of any one of embodiments 24 to 51, wherein the loading dose is administered with food.
54. The method of any one of embodiments 24 to 53, wherein the at least one weekly maintenance dose is orally administered.
55. The method of any one of embodiments 24 to 54, wherein the at least one weekly maintenance dose is administered in at least one tablet.
56. The method of any one of embodiments 24 to 54, wherein the at least one weekly maintenance dose is administered in an oral suspension.
57. The method of any one of embodiments 24 to 56, wherein the at least one weekly maintenance dose is administered without food.
58. The method of any one of embodiments 24 to 56, wherein the at least one weekly maintenance dose is administered with food.
59. The method of any one of embodiments 24 to 58, further comprising administering at least one additional active pharmaceutical ingredient.
60. The method of embodiment 59, wherein the at least one additional active pharmaceutical ingredient is chosen from antipsychotics.
61. The method of any one of embodiments 24 to 60, comprising treating at least one negative symptom of schizophrenia.
62. The method of embodiment 61, wherein the at least one negative symptom of schizophrenia is chosen from anhedonia, loss of motivation, and reduced interest in social interaction.
63. The method of any one of embodiments 24 to 62, comprising treating at least one cognitive symptom of schizophrenia.
64. The method of embodiment 63, wherein the at least one cognitive symptom of schizophrenia is chosen from impaired verbal memory, impaired working memory, impaired motor function, impaired attention and processing speed, impaired verbal fluency, and impaired executive function.
65. The method of any one of embodiments 24 to 64, comprising modulating reward anticipation related brain activity in the patient.
66. The method of any one of embodiments 24 to 65, comprising modulating cerebral blood flow in the patient.
67. The method of any one of embodiments 24 to 66, comprising increasing ventral striatal activity during reward anticipation in the patient.
68. The method of any one of embodiments 24 to 67, comprising modulating dopamine release in the patient.
69. A pharmaceutical composition comprising:
more than 80 mg of at least one compound chosen from Compound (I):

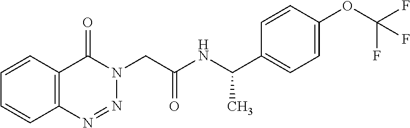 [SMILES: C[C@H](NC(=O)Cn1nnc2ccccc2c1=O)c1ccc(OC(F)(F)F)cc1]

and pharmaceutically acceptable salts thereof, and
at least one pharmaceutically acceptable excipient.
70. The pharmaceutical composition of embodiment 70, comprising more than 100 mg of at least one compound chosen from Compound (I) and pharmaceutically acceptable salts thereof.
71. The pharmaceutical composition of embodiment 69 or 70, comprising 120 mg of at least one compound chosen from Compound (I) and pharmaceutically acceptable salts thereof.
72. The pharmaceutical composition of embodiment 69 or 70, comprising 160 mg of at least one compound chosen from Compound (I) and pharmaceutically acceptable salts thereof.
73. The pharmaceutical composition of any one of embodiments 69 to 72, wherein the pharmaceutical composition is in the form of at least one tablet (e.g., 1, 2, 3, or 4 tablets).
74. The pharmaceutical composition of any one of embodiments 69 to 72, wherein the pharmaceutical composition is in the form of an oral suspension.

Some embodiments of this disclosure relate to methods of treating schizophrenia comprising administering to a patient in need thereof more than 80 mg of at least one compound chosen from Compound (I) and pharmaceutically acceptable salts thereof.

In some embodiments, more than 85 mg of at least one compound chosen from Compound (I) and pharmaceutically acceptable salts thereof is administered. In some embodiments, more than 90 mg of at least one compound chosen from Compound (I) and pharmaceutically acceptable salts thereof is administered. In some embodiments, more than 95 mg of at least one compound chosen from Compound (I) and pharmaceutically acceptable salts thereof is administered. In some embodiments, more than 100 mg of at least one compound chosen from Compound (I) and pharmaceutically acceptable salts thereof is administered. In some embodiments, more than 105 mg of at least one compound chosen from Compound (I) and pharmaceutically acceptable salts thereof is administered. In some embodiments, more than 110 mg of at least one compound chosen from Compound (I) and pharmaceutically acceptable salts thereof is administered. In some embodiments, more than 115 mg of at least one compound chosen from Compound (I) and pharmaceutically acceptable salts thereof is administered. In some embodiments, more than 120 mg of at least one compound chosen from Compound (I) and pharmaceutically acceptable salts thereof is administered. In some embodiments, more than 125 mg of at least one compound chosen from Compound (I) and pharmaceutically acceptable salts thereof is administered. In some embodiments, more than 130 mg of at least one compound chosen from Compound (I) and pharmaceutically acceptable salts thereof is administered. In some embodiments, more than 135 mg of at least one compound chosen from Compound (I) and pharmaceutically acceptable salts thereof is administered. In some embodiments, more than 140 mg of at least one compound chosen from Compound (I) and pharmaceutically acceptable salts thereof is administered. In some embodiments, more than 145 mg of at least one compound chosen from Compound (I) and pharmaceutically acceptable salts thereof is administered. In some embodiments, more than 150 mg of at least one compound chosen from Compound (I) and pharmaceutically acceptable salts thereof is administered. In some embodiments, more than 155 mg of at least one compound chosen from Compound (I) and pharmaceutically acceptable salts thereof is administered. In some embodiments, no more than 160 mg of at least one compound chosen from Compound (I) and pharmaceutically acceptable salts thereof is administered.

In some embodiments, less than 85 mg of at least one compound chosen from Compound (I) and pharmaceutically acceptable salts thereof is administered. In some embodiments, less than 90 mg of at least one compound chosen from Compound (I) and pharmaceutically acceptable salts thereof is administered. In some embodiments, less than 95 mg of at least one compound chosen from Compound (I) and pharmaceutically acceptable salts thereof is administered. In some embodiments, less than 100 mg of at least one compound chosen from Compound (I) and pharmaceutically acceptable salts thereof is administered. In some embodiments, less than 105 mg of at least one compound chosen from Compound (I) and pharmaceutically acceptable salts thereof is administered. In some embodiments, less than 110 mg of at least one compound chosen from Compound (I) and pharmaceutically acceptable salts thereof is administered. In some embodiments, less than 115 mg of at least one compound chosen from Compound (I) and pharmaceutically acceptable salts thereof is administered. In some embodiments, less than 120 mg of at least one compound chosen from Compound (I) and pharmaceutically acceptable salts thereof is administered. In some embodiments, less than 125 mg of at least one compound chosen from Compound (I) and pharmaceutically acceptable salts thereof is administered. In some embodiments, less than 130 mg of at least one compound chosen from Compound (I) and pharmaceutically acceptable salts thereof is administered. In some embodiments, less than 135 mg of at least one compound chosen from Compound (I) and pharmaceutically acceptable salts thereof is administered. In some embodiments, less than 140 mg of at least one compound chosen from Compound (I) and pharmaceutically acceptable salts thereof is administered. In some embodiments, less than 145 mg of at least one compound chosen from Compound (I) and pharmaceutically acceptable salts thereof is administered. In some embodiments, less than 150 mg of at least one compound chosen from Compound (I) and pharmaceutically acceptable salts thereof is administered. In some embodiments, less than 155 mg of at least one compound chosen from Compound (I) and pharmaceutically acceptable salts thereof is administered. In some embodiments, less than 160 mg of at least one compound chosen from Compound (I) and pharmaceutically acceptable salts thereof is administered. In some embodiments, less than 165 mg of at least one compound chosen from Compound (I) and pharmaceutically acceptable salts thereof is administered. In some embodiments, less than 170 mg of at least one compound chosen from Compound (I) and pharmaceutically acceptable salts thereof is administered.

In some embodiments, 85 mg of at least one compound chosen from Compound (I) and pharmaceutically acceptable salts thereof is administered. In some embodiments, 90 mg of at least one compound chosen from Compound (I) and pharmaceutically acceptable salts thereof is administered. In some embodiments, 95 mg of at least one compound chosen from Compound (I) and pharmaceutically acceptable salts thereof is administered. In some embodiments, 100 mg of at least one compound chosen from Compound (I) and pharmaceutically acceptable salts thereof is administered. In some embodiments, 105 mg of at least one compound chosen from Compound (I) and pharmaceutically acceptable salts thereof is administered. In some embodiments, 110 mg of at least one compound chosen from Compound (I) and pharmaceutically acceptable salts thereof is administered. In some embodiments, 115 mg of at least one compound chosen from Compound (I) and pharmaceutically acceptable salts thereof is administered. In some embodiments, 120 mg of at least one compound chosen from Compound (I) and pharmaceutically acceptable salts thereof is administered. In some embodiments, 125 mg of at least one compound chosen from Compound (I) and pharmaceutically acceptable salts thereof is administered. In some embodiments, 130 mg of at least one compound chosen from Compound (I) and pharmaceutically acceptable salts thereof is administered. In some embodiments, 135 mg of at least one compound chosen from Compound (I) and pharmaceutically acceptable salts thereof is administered. In some embodiments, 140 mg of at least one compound chosen from Compound (I) and pharmaceutically acceptable salts thereof is administered. In some embodiments, 145 mg of at least one compound chosen from Compound (I) and pharmaceutically acceptable salts thereof is administered. In some embodiments, 150 mg of at least one compound chosen from Compound (I) and pharmaceutically acceptable salts thereof is administered. In some embodiments, 155 mg of at least one compound chosen from Compound (I) and pharmaceutically acceptable salts thereof is administered. In some embodiments, 160 mg of at least one compound chosen from Compound (I) and pharmaceutically acceptable salts thereof is administered.

In some embodiments, the at least one compound chosen from Compound (I) and pharmaceutically acceptable salts thereof is effective to achieve a mean Cₘₐₓ in the patient of at least 500 ng/mL. In some embodiments, the at least one compound chosen from Compound (I) and pharmaceutically acceptable salts thereof is effective to achieve a mean Cₘₐₓ in the patient of at least 550 ng/mL. In some embodiments, the at least one compound chosen from Compound (I) and pharmaceutically acceptable salts thereof is effective to achieve a mean Cₘₐₓ in the patient of at least 600 ng/mL. In some embodiments, the at least one compound chosen from Compound (I) and pharmaceutically acceptable salts thereof is effective to achieve a mean Cₘₐₓ in the patient of at least 650 ng/mL. In some embodiments, the at least one compound chosen from Compound (I) and pharmaceutically acceptable salts thereof is effective to achieve a mean Cₘₐₓ in the patient of at least 700 ng/mL. In some embodiments, the at least one compound chosen from Compound (I) and pharmaceutically acceptable salts thereof is effective to achieve a mean Cₘₐₓ in the patient of at least 750 ng/mL. In some embodiments, the at least one compound chosen from Compound (I) and pharmaceutically acceptable salts thereof is effective to achieve a mean Cₘₐₓ in the patient of at least 800 ng/mL. In some embodiments, the at least one compound chosen from Compound (I) and pharmaceutically acceptable salts thereof is effective to achieve a mean Cₘₐₓ in the patient of at least 850 ng/mL. In some embodiments, the at least one compound chosen from Compound (I) and pharmaceutically acceptable salts thereof is effective to achieve a mean Cₘₐₓ in the patient of at least 900 ng/mL. In some embodiments, the at least one compound chosen from Compound (I) and pharmaceutically acceptable salts thereof is effective to achieve a mean Cₘₐₓ in the patient of at least 950 ng/mL. In some embodiments, the at least one compound chosen from Compound (I) and pharmaceutically acceptable salts thereof is effective to achieve a mean Cₘₐₓ in the patient of at least 1000 ng/mL. In some embodiments, the at least one compound chosen from Compound (I) and pharmaceutically acceptable salts thereof is effective to achieve a mean Cₘₐₓ in the patient of at least 1050 ng/mL. In some embodiments, the at least one compound chosen from Compound (I) and pharmaceutically acceptable salts thereof is effective to achieve a mean Cₘₐₓ in the patient of at least 1100 ng/mL. In some embodiments, the at least one compound chosen from Compound (I) and pharmaceutically acceptable salts thereof is effective to achieve a mean Cₘₐₓ in the patient of at least 1150 ng/mL. In some embodiments, the at least one compound chosen from Compound (I) and pharmaceutically acceptable salts thereof is effective to achieve a mean Cₘₐₓ in the patient of at least 1200 ng/mL. In some embodiments, the at least one compound chosen from Compound (I) and pharmaceutically acceptable salts thereof is effective to achieve a mean Cₘₐₓ in the patient of at least 1250 ng/mL. In some embodiments, the at least one compound chosen from Compound (I) and pharmaceutically acceptable salts thereof is effective to achieve a mean Cₘₐₓ in the patient of at least 1300 ng/mL. In some embodiments, the at least one compound chosen from Compound (I) and pharmaceutically acceptable salts thereof is effective to achieve a mean Cₘₐₓ in the patient of at least 1350 ng/mL. In some embodiments, the at least one compound chosen from Compound (I) and pharmaceutically acceptable salts thereof is effective to achieve a mean Cₘₐₓ in the patient of at least 1400 ng/mL. In some embodiments, the at least one compound chosen from Compound (I) and pharmaceutically acceptable salts thereof is effective to achieve a mean Cₘₐₓ in the patient of at least 1450 ng/mL. In some embodiments, the at least one compound chosen from Compound (I) and pharmaceutically acceptable salts thereof is effective to achieve a mean Cₘₐₓ in the patient of at least 1500 ng/mL. In some embodiments, the at least one compound chosen from Compound (I) and pharmaceutically acceptable salts thereof is effective to achieve a mean Cₘₐₓ in the patient of at least 1550 ng/mL. In some embodiments, the at least one compound chosen from Compound (I) and pharmaceutically acceptable salts thereof is effective to achieve a mean Cₘₐₓ in the patient of at least 1600 ng/mL. In some embodiments, the at least one compound chosen from Compound (I) and pharmaceutically acceptable salts thereof is effective to achieve a mean Cₘₐₓ in the patient of at least 1650 ng/mL. In some embodiments, the at least one compound chosen from Compound (I) and pharmaceutically acceptable salts thereof is effective to achieve a mean Cₘₐₓ in the patient of at least 1700 ng/mL. In some embodiments, the at least one compound chosen from Compound (I) and pharmaceutically acceptable salts thereof is effective to achieve a mean Cₘₐₓ in the patient of at least 1750 ng/mL. In some embodiments, the at least one compound chosen from Compound (I) and pharmaceutically acceptable salts thereof is effective to achieve a mean Cₘₐₓ in the patient of at least 1800 ng/mL. In some embodiments, the at least one compound chosen from Compound (I) and pharmaceutically acceptable salts thereof is effective to achieve a mean Cₘₐₓ in the patient of at least 1850 ng/mL. In some embodiments, the at least one compound chosen from Compound (I) and pharmaceutically acceptable salts thereof is effective to achieve a mean Cₘₐₓ in the patient of at least 1900 ng/mL. In some embodiments, the at least one compound chosen from Compound (I) and pharmaceutically acceptable salts thereof is effective to achieve a mean Cₘₐₓ in the patient of at least 1950 ng/mL. In some embodiments, the at least one compound chosen from Compound (I) and pharmaceutically acceptable salts thereof is effective to achieve a mean Cₘₐₓ in the patient of at least 2000 ng/mL. In some embodiments, the at least one compound chosen from Compound (I) and pharmaceutically acceptable salts thereof is effective to achieve a mean Cₘₐₓ in the patient of at least 2050 ng/mL. In some embodiments, the at least one compound chosen from Compound (I) and pharmaceutically acceptable salts thereof is effective to achieve a mean Cₘₐₓ in the patient of at least 2100 ng/mL. In some embodiments, the at least one compound chosen from Compound (I) and pharmaceutically acceptable salts thereof is effective to achieve a mean Cₘₐₓ in the patient of at least 2150 ng/mL. In some embodiments, the at least one compound chosen from Compound (I) and pharmaceutically acceptable salts thereof is effective to achieve a mean Cₘₐₓ in the patient of at least 2200 ng/mL. In some embodiments, the at least one compound chosen from Compound (I) and pharmaceutically acceptable salts thereof is effective to achieve a mean Cₘₐₓ in the patient of at least 2250 ng/mL. In some embodiments, the at least one compound chosen from Compound (I) and pharmaceutically acceptable salts thereof is effective to achieve a mean Cₘₐₓ in the patient of at least 2300 ng/mL. In some embodiments, the at least one compound chosen from Compound (I) and pharmaceutically acceptable salts thereof is effective to achieve a mean Cₘₐₓ in the patient of at least 2350 ng/mL. In some embodiments, the at least one compound chosen from Compound (I) and pharmaceutically acceptable salts thereof is effective to achieve a mean Cₘₐₓ in the patient of at least 2400 ng/mL. In some embodiments, the at least one compound chosen from Compound (I) and pharmaceutically acceptable salts thereof is effective to achieve a mean Cₘₐₓ in the patient of at least 2450 ng/mL.

In some embodiments, the at least one compound chosen from Compound (I) and pharmaceutically acceptable salts thereof is effective to achieve a mean Cₘₐₓ in the patient of not more than 550 ng/mL. In some embodiments, the at least one compound chosen from Compound (I) and pharmaceutically acceptable salts thereof is effective to achieve a mean Cₘₐₓ in the patient of not more than 600 ng/mL. In some embodiments, the at least one compound chosen from Compound (I) and pharmaceutically acceptable salts thereof is effective to achieve a mean Cₘₐₓ in the patient of not more than 650 ng/mL. In some embodiments, the at least one compound chosen from Compound (I) and pharmaceutically acceptable salts thereof is effective to achieve a mean Cₘₐₓ in the patient of not more than 700 ng/mL. In some embodiments, the at least one compound chosen from Compound (I) and pharmaceutically acceptable salts thereof is effective to achieve a mean Cₘₐₓ in the patient of not more than 750 ng/mL. In some embodiments, the at least one compound chosen from Compound (I) and pharmaceutically acceptable salts thereof is effective to achieve a mean Cₘₐₓ in the patient of not more than 800 ng/mL. In some embodiments, the at least one compound chosen from Compound (I) and pharmaceutically acceptable salts thereof is effective to achieve a mean Cₘₐₓ in the patient of not more than 850 ng/mL. In some embodiments, the at least one compound chosen from Compound (I) and pharmaceutically acceptable salts thereof is effective to achieve a mean Cₘₐₓ in the patient of not more than 900 ng/mL. In some embodiments, the at least one compound chosen from Compound (I) and pharmaceutically acceptable salts thereof is effective to achieve a mean Cₘₐₓ in the patient of not more than 950 ng/mL. In some embodiments, the at least one compound chosen from Compound (I) and pharmaceutically acceptable salts thereof is effective to achieve a mean Cₘₐₓ in the patient of not more than 1000 ng/mL. In some embodiments, the at least one compound chosen from Compound (I) and pharmaceutically acceptable salts thereof is effective to achieve a mean Cₘₐₓ in the patient of not more than 1050 ng/mL. In some embodiments, the at least one compound chosen from Compound (I) and pharmaceutically acceptable salts thereof is effective to achieve a mean Cₘₐₓ in the patient of not more than 1100 ng/mL. In some embodiments, the at least one compound chosen from Compound (I) and pharmaceutically acceptable salts thereof is effective to achieve a mean Cₘₐₓ in the patient of not more than 1150 ng/mL. In some embodiments, the at least one compound chosen from Compound (I) and pharmaceutically acceptable salts thereof is effective to achieve a mean Cₘₐₓ in the patient of not more than 1200 ng/mL. In some embodiments, the at least one compound chosen from Compound (I) and pharmaceutically acceptable salts thereof is effective to achieve a mean Cₘₐₓ in the patient of not more than 1250 ng/mL. In some embodiments, the at least one compound chosen from Compound (I) and pharmaceutically acceptable salts thereof is effective to achieve a mean Cₘₐₓ in the patient of not more than 1300 ng/mL. In some embodiments, the at least one compound chosen from Compound (I) and pharmaceutically acceptable salts thereof is effective to achieve a mean Cₘₐₓ in the patient of not more than 1350 ng/mL. In some embodiments, the at least one compound chosen from Compound (I) and pharmaceutically acceptable salts thereof is effective to achieve a mean Cₘₐₓ in the patient of not more than 1400 ng/mL. In some embodiments, the at least one compound chosen from Compound (I) and pharmaceutically acceptable salts thereof is effective to achieve a mean Cₘₐₓ in the patient of not more than 1450 ng/mL. In some embodiments, the at least one compound chosen from Compound (I) and pharmaceutically acceptable salts thereof is effective to achieve a mean Cₘₐₓ in the patient of not more than 1500 ng/mL. In some embodiments, the at least one compound chosen from Compound (I) and pharmaceutically acceptable salts thereof is effective to achieve a mean Cₘₐₓ in the patient of not more than 1550 ng/mL. In some embodiments, the at least one compound chosen from Compound (I) and pharmaceutically acceptable salts thereof is effective to achieve a mean Cₘₐₓ in the patient of not more than 1600 ng/mL. In some embodiments, the at least one compound chosen from Compound (I) and pharmaceutically acceptable salts thereof is effective to achieve a mean Cₘₐₓ in the patient of not more than 1650 ng/mL. In some embodiments, the at least one compound chosen from Compound (I) and pharmaceutically acceptable salts thereof is effective to achieve a mean Cₘₐₓ in the patient of not more than 1700 ng/mL. In some embodiments, the at least one compound chosen from Compound (I) and pharmaceutically acceptable salts thereof is effective to achieve a mean Cₘₐₓ in the patient of not more than 1750 ng/mL. In some embodiments, the at least one compound chosen from Compound (I) and pharmaceutically acceptable salts thereof is effective to achieve a mean Cₘₐₓ in the patient of not more than 1800 ng/mL. In some embodiments, the at least one compound chosen from Compound (I) and pharmaceutically acceptable salts thereof is effective to achieve a mean Cₘₐₓ in the patient of not more than 1850 ng/mL. In some embodiments, the at least one compound chosen from Compound (I) and pharmaceutically acceptable salts thereof is effective to achieve a mean Cₘₐₓ in the patient of not more than 1900 ng/mL. In some embodiments, the at least one compound chosen from Compound (I) and pharmaceutically acceptable salts thereof is effective to achieve a mean Cₘₐₓ in the patient of not more than 1950 ng/mL. In some embodiments, the at least one compound chosen from Compound (I) and pharmaceutically acceptable salts thereof is effective to achieve a mean Cₘₐₓ in the patient of not more than 2000 ng/mL. In some embodiments, the at least one compound chosen from Compound (I) and pharmaceutically acceptable salts thereof is effective to achieve a mean Cₘₐₓ in the patient of not more than 2050 ng/mL. In some embodiments, the at least one compound chosen from Compound (I) and pharmaceutically acceptable salts thereof is effective to achieve a mean Cₘₐₓ in the patient of not more than 2100 ng/mL. In some embodiments, the at least one compound chosen from Compound (I) and pharmaceutically acceptable salts thereof is effective to achieve a mean Cₘₐₓ in the patient of not more than 2150 ng/mL. In some embodiments, the at least one compound chosen from Compound (I) and pharmaceutically acceptable salts thereof is effective to achieve a mean Cₘₐₓ in the patient of not more than 2200 ng/mL. In some embodiments, the at least one compound chosen from Compound (I) and pharmaceutically acceptable salts thereof is effective to achieve a mean Cₘₐₓ in the patient of not more than 2250 ng/mL. In some embodiments, the at least one compound chosen from Compound (I) and pharmaceutically acceptable salts thereof is effective to achieve a mean Cₘₐₓ in the patient of not more than 2300 ng/mL. In some embodiments, the at least one compound chosen from Compound (I) and pharmaceutically acceptable salts thereof is effective to achieve a mean Cₘₐₓ in the patient of not more than 2350 ng/mL. In some embodiments, the at least one compound chosen from Compound (I) and pharmaceutically acceptable salts thereof is effective to achieve a mean Cₘₐₓ in the patient of not more than 2400 ng/mL. In some embodiments, the at least one compound chosen from Compound (I) and pharmaceutically acceptable salts thereof is effective to achieve a mean Cₘₐₓ in the patient of not more than 2450 ng/mL. In some embodiments, the at least one compound chosen from Compound (I) and pharmaceutically acceptable salts thereof is effective to achieve a mean Cₘₐₓ in the patient of not more than 2500 ng/mL.

In some embodiments, the at least one compound chosen from Compound (I) and pharmaceutically acceptable salts thereof is effective to achieve a mean Cₘₐₓ in the patient from 500 ng/mL to 2500 ng/mL.

In some embodiments, the at least one compound chosen from Compound (I) and pharmaceutically acceptable salts thereof is effective to achieve a mean Cₘₐₓ in the patient from 50% to 150% of 1267 ng/mL. In some embodiments, the at least one compound chosen from Compound (I) and pharmaceutically acceptable salts thereof is effective to achieve a mean Cₘₐₓ in the patient from 55% to 145% of 1267 ng/mL. In some embodiments, the at least one compound chosen from Compound (I) and pharmaceutically acceptable salts thereof is effective to achieve a mean Cₘₐₓ in the patient from 60% to 140% of 1267 ng/mL. In some embodiments, the at least one compound chosen from Compound (I) and pharmaceutically acceptable salts thereof is effective to achieve a mean Cₘₐₓ in the patient from 65% to 135% of 1267 ng/mL. In some embodiments, the at least one compound chosen from Compound (I) and pharmaceutically acceptable salts thereof is effective to achieve a mean Cₘₐₓ in the patient from 70% to 130% of 1267 ng/mL. In some embodiments, the at least one compound chosen from Compound (I) and pharmaceutically acceptable salts thereof is effective to achieve a mean Cₘₐₓ in the patient from 75% to 125% of 1267 ng/mL. In some embodiments, the at least one compound chosen from Compound (I) and pharmaceutically acceptable salts thereof is effective to achieve a mean Cₘₐₓ in the patient from 80% to 120% of 1267 ng/mL. In some embodiments, the at least one compound chosen from Compound (I) and pharmaceutically acceptable salts thereof is effective to achieve a mean Cₘₐₓ in the patient from 85% to 115% of 1267 ng/mL. In some embodiments, the at least one compound chosen from Compound (I) and pharmaceutically acceptable salts thereof is effective to achieve a mean Cₘₐₓ in the patient from 90% to 110% of 1267 ng/mL. In some embodiments, the at least one compound chosen from Compound (I) and pharmaceutically acceptable salts thereof is effective to achieve a mean Cₘₐₓ in the patient from 95% to 105% of 1267 ng/mL.

In some embodiments, the mean t_{1/2z} in the patient is from 50 h to 1000 h. In some embodiments, the mean t_{1/2z} in the patient is from 100 h to 1000 h. In some embodiments, the mean t_{1/2z} in the patient is from 50 h to 900 h. In some embodiments, the mean t_{1/2z} in the patient is from 50 h to 800 h. In some embodiments, the mean t_{1/2z} in the patient is from 50 h to 700 h. In some embodiments, the mean t_{1/2z} in the patient is from 50 h to 600 h. In some embodiments, the mean t_{1/2z} in the patient is from 50 h to 500 h. In some embodiments, the mean t_{1/2z} in the patient is from 50 h to 400 h. In some embodiments, the mean t_{1/2z} in the patient is from 100 h to 500 h. In some embodiments, the mean t_{1/2z} in the patient is from 100 h to 400 h. In some embodiments, the mean t_{1/2z} in the patient is from 100 h to 300 h. In some embodiments, the mean t_{1/2z} in the patient is from 200 h to 300 h.

In some embodiments, the mean t_{1/2z} in the patient is at least 100 h. In some embodiments, the mean t_{1/2z} in the patient is at least 125 h. In some embodiments, the mean t_{1/2z} in the patient is at least 150 h. In some embodiments, the mean t_{1/2z} in the patient is at least 175 h. In some embodiments, the mean t_{1/2z} at steady state in the patient is at least 200 h.

In some embodiments, the at least one compound chosen from Compound (I) and pharmaceutically acceptable salts thereof is orally administered.

In some embodiments, the at least one compound chosen from Compound (I) and pharmaceutically acceptable salts thereof is administered in at least one tablet. In some embodiments, the at least one compound chosen from Compound (I) and pharmaceutically acceptable salts thereof is administered in at least one immediate release tablet. In some embodiments, the at least one compound chosen from Compound (I) and pharmaceutically acceptable salts thereof is administered in at least one immediate release tablet further comprising mannitol and microcrystalline cellulose.

In some embodiments, the at least one compound chosen from Compound (I) and pharmaceutically acceptable salts thereof is administered in more than one tablet (e.g., 2, 3, or 4 tablets). In some embodiments, the at least one compound chosen from Compound (I) and pharmaceutically acceptable salts thereof is administered in more than one immediate release tablet (e.g., 2, 3, or 4 immediate release tablets). In some embodiments, the at least one compound chosen from Compound (I) and pharmaceutically acceptable salts thereof is administered in more than one immediate release tablet (e.g., 2, 3, or 4 immediate release tablets) further comprising mannitol and microcrystalline cellulose.

In some embodiments, the at least one compound chosen from Compound (I) and pharmaceutically acceptable salts thereof is administered in an oral suspension. In some embodiments, the at least one compound chosen from Compound (I) and pharmaceutically acceptable salts thereof is administered in an oral suspension further comprising Tween and methylcellulose.

In some embodiments, the at least one compound chosen from Compound (I) and pharmaceutically acceptable salts thereof is administered with water.

In some embodiments, the at least one compound chosen from Compound (I) and pharmaceutically acceptable salts thereof is administered without food. In some embodiments, the patient is in a fasted state. In some embodiments, the patient has fasted for at least 10 h prior to the administration of the at least one compound chosen from Compound (I) and pharmaceutically acceptable salts thereof.

In some embodiments, the at least one compound chosen from Compound (I) and pharmaceutically acceptable salts thereof is administered with food. In some embodiments, the at least one compound chosen from Compound (I) and pharmaceutically acceptable salts thereof is administered with a high-fat, high-calorie meal. In some embodiments, the patient is in a fed state. In some embodiments, the patient ate at least 30 min prior to the administration of the at least one compound chosen from Compound (I) and pharmaceutically acceptable salts thereof.

In some embodiments, the methods further comprise administering at least one additional active pharmaceutical ingredient. In some embodiments, the at least one additional active pharmaceutical ingredient is chosen from sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, mGlu2/3 agonists, 5HT-2 antagonists, PDE10 antagonists, and GlyT1 inhibitors.

In some embodiments, the at least one additional active pharmaceutical ingredient is chosen from antipsychotics.

In some embodiments, the at least one additional active pharmaceutical ingredient is chosen from first-generation antipsychotics. In some embodiments, the at least one additional active pharmaceutical ingredient is chosen from chlorpromazine, fluphenazine, haloperidol, and perphenazine.

In some embodiments, the at least one additional active pharmaceutical ingredient is chosen from second-generation antipsychotics. In some embodiments, the at least one additional active pharmaceutical ingredient is chosen from aripiprazole, asenapine, brexpiprazole, cariprazine, clozapine, iloperidone, lurasidone, olanzapine, paliperidone, quetiapine, risperidone, and ziprasidone.

In some embodiments, the at least one additional active pharmaceutical ingredient is chosen from long-acting injectable antipsychotics. In some embodiments, the at least one additional active pharmaceutical ingredient is chosen from aripiprazole, fluphenazine decanoate, haloperidol decanoate, paliperidone, and risperidone.

In some embodiments, the methods comprise treating at least one negative symptom of schizophrenia. In some embodiments, the at least one negative symptom of schizophrenia is chosen from anhedonia, loss of motivation, and reduced interest in social interaction. In some embodiments, the at least one negative symptom of schizophrenia is anhedonia. In some embodiments, the at least one negative symptom of schizophrenia is loss of motivation. In some embodiments, the at least one negative symptom of schizophrenia is reduced interest in social interaction.

In some embodiments, the methods comprise treating at least one cognitive symptom of schizophrenia. In some embodiments, the at least one cognitive symptom of schizophrenia is chosen from impaired verbal memory, impaired working memory, impaired motor function, impaired attention and processing speed, impaired verbal fluency, and impaired executive function. In some embodiments, the at least one cognitive symptom of schizophrenia is impaired verbal memory. In some embodiments, the at least one cognitive symptom of schizophrenia is impaired working memory. In some embodiments, the at least one cognitive symptom of schizophrenia is impaired motor function. In some embodiments, the at least one cognitive symptom of schizophrenia is impaired attention and processing speed. In some embodiments, the at least one cognitive symptom of schizophrenia is impaired verbal fluency. In some embodiments, the at least one cognitive symptom of schizophrenia is impaired executive function.

In some embodiments, the methods comprise modulating reward anticipation related brain activity in the patient.

In some embodiments, the methods comprise modulating cerebral blood flow in the patient.

In some embodiments, the methods comprise increasing ventral striatal activity during reward anticipation in the patient.

In some embodiments, the methods comprise modulating dopamine release in the patient. In some embodiments, the methods comprise reducing dopamine release in the patient. In some embodiments, the methods comprise reducing dopamine release in the patient following an exposure to a stimulant.

Some embodiments of this disclosure relate to methods of treating schizophrenia comprising administering to a patient in need thereof: a loading dose comprising more than 20 mg of at least one compound chosen from Compound (I) and pharmaceutically acceptable salts thereof, and at least one weekly maintenance dose comprising at least one compound chosen from Compound (I) and pharmaceutically acceptable salts thereof.

In some embodiments, the at least one weekly maintenance dose is administered 5 to 9 days after the loading dose. In some embodiments, the at least one weekly maintenance dose is administered 6 to 8 days after the loading dose. In some embodiments, the at least one weekly maintenance dose is administered 5 days after the loading dose. In some embodiments, the at least one weekly maintenance dose is administered 6 days after the loading dose. In some embodiments, the at least one weekly maintenance dose is administered 7 days after the loading dose. In some embodiments, the at least one weekly maintenance dose is administered 8 days after the loading dose. In some embodiments, the at least one weekly maintenance dose is administered 9 days after the loading dose.

In some embodiments, a first weekly maintenance dose is administered 7 days after the loading dose, and a second weekly maintenance dose is administered 14 days after the loading dose.

In some embodiments, a first weekly maintenance dose is administered 5 days to 9 days after the loading dose, and a second weekly maintenance dose is administered 12 days to 16 days after the loading dose.

In some embodiments, a first weekly maintenance dose is administered 7 days after the loading dose, a second weekly maintenance dose is administered 14 days after the loading dose, and a third weekly maintenance dose is administered 21 days after the loading dose.

In some embodiments, a first weekly maintenance dose is administered 5 days to 9 days after the loading dose, a second weekly maintenance dose is administered 12 days to 16 days after the loading dose, and a third weekly maintenance dose is administered 19 days to 23 days after the loading dose.

In some embodiments, each weekly maintenance dose is administered 5 days to 9 days after the prior dose comprising at least one compound chosen from Compound (I) and pharmaceutically acceptable salts thereof.

In some embodiments, the loading dose comprises more than 30 mg of at least one compound chosen from Compound (I) and pharmaceutically acceptable salts thereof. In some embodiments, the loading dose comprises more than 40 mg of at least one compound chosen from Compound (I) and pharmaceutically acceptable salts thereof. In some embodiments, the loading dose comprises more than 50 mg of at least one compound chosen from Compound (I) and pharmaceutically acceptable salts thereof. In some embodiments, the loading dose comprises more than 60 mg of at least one compound chosen from Compound (I) and pharmaceutically acceptable salts thereof. In some embodiments, the loading dose comprises more than 70 mg of at least one compound chosen from Compound (I) and pharmaceutically acceptable salts thereof. In some embodiments, the loading dose comprises more than 80 mg of at least one compound chosen from Compound (I) and pharmaceutically acceptable salts thereof. In some embodiments, the loading dose comprises more than 90 mg of at least one compound chosen from Compound (I) and pharmaceutically acceptable salts thereof. In some embodiments, the loading dose comprises more than 100 mg of at least one compound chosen from Compound (I) and pharmaceutically acceptable salts thereof. In some embodiments, the loading dose comprises more than 110 mg of at least one compound chosen from Compound (I) and pharmaceutically acceptable salts thereof. In some embodiments, the loading dose comprises more than 120 mg of at least one compound chosen from Compound (I) and pharmaceutically acceptable salts thereof. In some embodiments, the loading dose comprises more than 130 mg of at least one compound chosen from Compound (I) and pharmaceutically acceptable salts thereof. In some embodiments, the loading dose comprises more than 140 mg of at least one compound chosen from Compound (I) and pharmaceutically acceptable salts thereof. In some embodiments, the loading dose comprises more than 150 mg of at least one compound chosen from Compound (I) and pharmaceutically acceptable salts thereof.

In some embodiments, the loading dose comprises 30 mg of at least one compound chosen from Compound (I) and pharmaceutically acceptable salts thereof. In some embodiments, the loading dose comprises 40 mg of at least one compound chosen from Compound (I) and pharmaceutically acceptable salts thereof. In some embodiments, the loading dose comprises 50 mg of at least one compound chosen from Compound (I) and pharmaceutically acceptable salts thereof. In some embodiments, the loading dose comprises 60 mg of at least one compound chosen from Compound (I) and pharmaceutically acceptable salts thereof. In some embodiments, the loading dose comprises 70 mg of at least one compound chosen from Compound (I) and pharmaceutically acceptable salts thereof. In some embodiments, the loading dose comprises 80 mg of at least one compound chosen from Compound (I) and pharmaceutically acceptable salts thereof. In some embodiments, the loading dose comprises 90 mg of at least one compound chosen from Compound (I) and pharmaceutically acceptable salts thereof. In some embodiments, the loading dose comprises 100 mg of at least one compound chosen from Compound (I) and pharmaceutically acceptable salts thereof. In some embodiments, the loading dose comprises 110 mg of at least one compound chosen from Compound (I) and pharmaceutically acceptable salts thereof. In some embodiments, the loading dose comprises 120 mg of at least one compound chosen from Compound (I) and pharmaceutically acceptable salts thereof. In some embodiments, the loading dose comprises 130 mg of at least one compound chosen from Compound (I) and pharmaceutically acceptable salts thereof. In some embodiments, the loading dose comprises 140 mg of at least one compound chosen from Compound (I) and pharmaceutically acceptable salts thereof. In some embodiments, the loading dose comprises 150 mg of at least one compound chosen from Compound (I) and pharmaceutically acceptable salts thereof. In some embodiments, the loading dose comprises 160 mg of at least one compound chosen from Compound (I) and pharmaceutically acceptable salts thereof.

In some embodiments, the loading dose is effective to achieve a mean Cₘₐₓ in the patient of at least 250 ng/mL. In some embodiments, the loading dose is effective to achieve a mean Cₘₐₓ in the patient of at least 300 ng/mL. In some embodiments, the loading dose is effective to achieve a mean Cₘₐₓ in the patient of at least 350 ng/mL. In some embodiments, the loading dose is effective to achieve a mean Cₘₐₓ in the patient of at least 400 ng/mL. In some embodiments, the loading dose is effective to achieve a mean Cₘₐₓ in the patient of at least 450 ng/mL. In some embodiments, the loading dose is effective to achieve a mean Cₘₐₓ in the patient of at least 500 ng/mL. In some embodiments, the loading dose is effective to achieve a mean Cₘₐₓ in the patient of at least 550 ng/mL. In some embodiments, the loading dose is effective to achieve a mean Cₘₐₓ in the patient of at least 600 ng/mL. In some embodiments, the loading dose is effective to achieve a mean Cₘₐₓ in the patient of at least 650 ng/mL. In some embodiments, the loading dose is effective to achieve a mean Cₘₐₓ in the patient of at least 700 ng/mL. In some embodiments, the loading dose is effective to achieve a mean Cₘₐₓ in the patient of at least 750 ng/mL. In some embodiments, the loading dose is effective to achieve a mean Cₘₐₓ in the patient of at least 800 ng/mL. In some embodiments, the loading dose is effective to achieve a mean Cₘₐₓ in the patient of at least 850 ng/mL. In some embodiments, the loading dose is effective to achieve a mean Cₘₐₓ in the patient of at least 900 ng/mL. In some embodiments, the loading dose is effective to achieve a mean Cₘₐₓ in the patient of at least 950 ng/mL. In some embodiments, the loading dose is effective to achieve a mean Cₘₐₓ in the patient of at least 1000 ng/mL. In some embodiments, the loading dose is effective to achieve a mean Cₘₐₓ in the patient of at least 1050 ng/mL. In some embodiments, the loading dose is effective to achieve a mean Cₘₐₓ in the patient of at least 1100 ng/mL. In some embodiments, the loading dose is effective to achieve a mean Cₘₐₓ in the patient of at least 1150 ng/mL. In some embodiments, the loading dose is effective to achieve a mean Cₘₐₓ in the patient of at least 1200 ng/mL. In some embodiments, the loading dose is effective to achieve a mean Cₘₐₓ in the patient of at least 1250 ng/mL. In some embodiments, the loading dose is effective to achieve a mean Cₘₐₓ in the patient of at least 1300 ng/mL. In some embodiments, the loading dose is effective to achieve a mean Cₘₐₓ in the patient of at least 1350 ng/mL. In some embodiments, the loading dose is effective to achieve a mean Cₘₐₓ in the patient of at least 1400 ng/mL. In some embodiments, the loading dose is effective to achieve a mean Cₘₐₓ in the patient of at least 1450 ng/mL. In some embodiments, the loading dose is effective to achieve a mean Cₘₐₓ in the patient of at least 1500 ng/mL. In some embodiments, the loading dose is effective to achieve a mean Cₘₐₓ in the patient of at least 1550 ng/mL. In some embodiments, the loading dose is effective to achieve a mean Cₘₐₓ in the patient of at least 1600 ng/mL. In some embodiments, the loading dose is effective to achieve a mean Cₘₐₓ in the patient of at least 1650 ng/mL. In some embodiments, the loading dose is effective to achieve a mean Cₘₐₓ in the patient of at least 1700 ng/mL. In some embodiments, the loading dose is effective to achieve a mean Cₘₐₓ in the patient of at least 1750 ng/mL. In some embodiments, the loading dose is effective to achieve a mean Cₘₐₓ in the patient of at least 1800 ng/mL. In some embodiments, the loading dose is effective to achieve a mean Cₘₐₓ in the patient of at least 1850 ng/mL. In some embodiments, the loading dose is effective to achieve a mean Cₘₐₓ in the patient of at least 1900 ng/mL. In some embodiments, the loading dose is effective to achieve a mean Cₘₐₓ in the patient of at least 1950 ng/mL. In some embodiments, the loading dose is effective to achieve a mean Cₘₐₓ in the patient of at least 2000 ng/mL. In some embodiments, the loading dose is effective to achieve a mean Cₘₐₓ in the patient of at least 2050 ng/mL. In some embodiments, the loading dose is effective to achieve a mean Cₘₐₓ in the patient of at least 2100 ng/mL. In some embodiments, the loading dose is effective to achieve a mean Cₘₐₓ in the patient of at least 2150 ng/mL. In some embodiments, the loading dose is effective to achieve a mean Cₘₐₓ in the patient of at least 2200 ng/mL. In some embodiments, the loading dose is effective to achieve a mean Cₘₐₓ in the patient of at least 2250 ng/mL. In some embodiments, the loading dose is effective to achieve a mean Cₘₐₓ in the patient of at least 2300 ng/mL. In some embodiments, the loading dose is effective to achieve a mean Cₘₐₓ in the patient of at least 2350 ng/mL. In some embodiments, the loading dose is effective to achieve a mean Cₘₐₓ in the patient of at least 2400 ng/mL. In some embodiments, the loading dose is effective to achieve a mean Cₘₐₓ in the patient of at least 2450 ng/mL.

In some embodiments, the loading dose is effective to achieve a mean Cₘₐₓ in the patient of not more than 300 ng/mL. In some embodiments, the loading dose is effective to achieve a mean Cₘₐₓ in the patient of not more than 350 ng/mL. In some embodiments, the loading dose is effective to achieve a mean Cₘₐₓ in the patient of not more than 400 ng/mL. In some embodiments, the loading dose is effective to achieve a mean Cₘₐₓ in the patient of not more than 450 ng/mL. In some embodiments, the loading dose is effective to achieve a mean Cₘₐₓ in the patient of not more than 500 ng/mL. In some embodiments, the loading dose is effective to achieve a mean Cₘₐₓ in the patient of not more than 550 ng/mL. In some embodiments, the loading dose is effective to achieve a mean Cₘₐₓ in the patient of not more than 600 ng/mL. In some embodiments, the loading dose is effective to achieve a mean Cₘₐₓ in the patient of not more than 650 ng/mL. In some embodiments, the loading dose is effective to achieve a mean Cₘₐₓ in the patient of not more than 700 ng/mL. In some embodiments, the loading dose is effective to achieve a mean Cₘₐₓ in the patient of not more than 750 ng/mL. In some embodiments, the loading dose is effective to achieve a mean Cₘₐₓ in the patient of not more than 800 ng/mL. In some embodiments, the loading dose is effective to achieve a mean Cₘₐₓ in the patient of not more than 850 ng/mL. In some embodiments, the loading dose is effective to achieve a mean Cₘₐₓ in the patient of not more than 900 ng/mL. In some embodiments, the loading dose is effective to achieve a mean Cₘₐₓ in the patient of not more than 950 ng/mL. In some embodiments, the loading dose is effective to achieve a mean Cₘₐₓ in the patient of not more than 1000 ng/mL. In some embodiments, the loading dose is effective to achieve a mean Cₘₐₓ in the patient of not more than 1050 ng/mL. In some embodiments, the loading dose is effective to achieve a mean Cₘₐₓ in the patient of not more than 1100 ng/mL. In some embodiments, the loading dose is effective to achieve a mean Cₘₐₓ in the patient of not more than 1150 ng/mL. In some embodiments, the loading dose is effective to achieve a mean Cₘₐₓ in the patient of not more than 1200 ng/mL. In some embodiments, the loading dose is effective to achieve a mean Cₘₐₓ in the patient of not more than 1250 ng/mL. In some embodiments, the loading dose is effective to achieve a mean Cₘₐₓ in the patient of not more than 1300 ng/mL. In some embodiments, the loading dose is effective to achieve a mean Cₘₐₓ in the patient of not more than 1350 ng/mL. In some embodiments, the loading dose is effective to achieve a mean Cₘₐₓ in the patient of not more than 1400 ng/mL. In some embodiments, the loading dose is effective to achieve a mean Cₘₐₓ in the patient of not more than 1450 ng/mL. In some embodiments, the loading dose is effective to achieve a mean Cₘₐₓ in the patient of not more than 1500 ng/mL. In some embodiments, the loading dose is effective to achieve a mean Cₘₐₓ in the patient of not more than 1550 ng/mL. In some embodiments, the loading dose is effective to achieve a mean Cₘₐₓ in the patient of not more than 1600 ng/mL. In some embodiments, the loading dose is effective to achieve a mean Cₘₐₓ in the patient of not more than 1650 ng/mL. In some embodiments, the loading dose is effective to achieve a mean Cₘₐₓ in the patient of not more than 1700 ng/mL. In some embodiments, the loading dose is effective to achieve a mean Cₘₐₓ in the patient of not more than 1750 ng/mL. In some embodiments, the loading dose is effective to achieve a mean Cₘₐₓ in the patient of not more than 1800 ng/mL. In some embodiments, the loading dose is effective to achieve a mean Cₘₐₓ in the patient of not more than 1850 ng/mL. In some embodiments, the loading dose is effective to achieve a mean Cₘₐₓ in the patient of not more than 1900 ng/mL. In some embodiments, the loading dose is effective to achieve a mean Cₘₐₓ in the patient of not more than 1950 ng/mL. In some embodiments, the loading dose is effective to achieve a mean Cₘₐₓ in the patient of not more than 2000 ng/mL. In some embodiments, the loading dose is effective to achieve a mean Cₘₐₓ in the patient of not more than 2050 ng/mL. In some embodiments, the loading dose is effective to achieve a mean Cₘₐₓ in the patient of not more than 2100 ng/mL. In some embodiments, the loading dose is effective to achieve a mean Cₘₐₓ in the patient of not more than 2150 ng/mL. In some embodiments, the loading dose is effective to achieve a mean Cₘₐₓ in the patient of not more than 2200 ng/mL. In some embodiments, the loading dose is effective to achieve a mean Cₘₐₓ in the patient of not more than 2250 ng/mL. In some embodiments, the loading dose is effective to achieve a mean Cₘₐₓ in the patient of not more than 2300 ng/mL. In some embodiments, the loading dose is effective to achieve a mean Cₘₐₓ in the patient of not more than 2350 ng/mL. In some embodiments, the loading dose is effective to achieve a mean Cₘₐₓ in the patient of not more than 2400 ng/mL. In some embodiments, the loading dose is effective to achieve a mean Cₘₐₓ in the patient of not more than 2450 ng/mL. In some embodiments, the loading dose is effective to achieve a mean Cₘₐₓ in the patient of not more than 2500 ng/mL.

In some embodiments, the loading dose is effective to achieve a mean Cₘₐₓ in the patient from 500 ng/mL to 2500 ng/mL.

In some embodiments, the loading dose is effective to achieve a mean Cₘₐₓ in the patient from 50% to 150% of 1267 ng/mL. In some embodiments, the loading dose is effective to achieve a mean Cₘₐₓ in the patient from 55% to 145% of 1267 ng/mL. In some embodiments, the loading dose is effective to achieve a mean Cₘₐₓ in the patient from 60% to 140% of 1267 ng/mL. In some embodiments, the loading dose is effective to achieve a mean Cₘₐₓ in the patient from 65% to 135% of 1267 ng/mL. In some embodiments, the loading dose is effective to achieve a mean Cₘₐₓ in the patient from 70% to 130% of 1267 ng/mL. In some embodiments, the loading dose is effective to achieve a mean Cₘₐₓ in the patient from 75% to 125% of 1267 ng/mL. In some embodiments, the loading dose is effective to achieve a mean Cₘₐₓ in the patient from 80% to 120% of 1267 ng/mL. In some embodiments, the loading dose is effective to achieve a mean Cₘₐₓ in the patient from 85% to 115% of 1267 ng/mL. In some embodiments, the loading dose is effective to achieve a mean Cₘₐₓ in the patient from 90% to 110% of 1267 ng/mL. In some embodiments, the loading dose is effective to achieve a mean Cₘₐₓ in the patient from 95% to 105% of 1267 ng/mL.

In some embodiments, the at least one weekly maintenance dose is half the loading dose. In some embodiments, the at least one weekly maintenance dose is from 30% to 70% of the loading dose. In some embodiments, the at least one weekly maintenance dose is from 35% to 65% of the loading dose. In some embodiments, the at least one weekly maintenance dose is from 40% to 60% of the loading dose. In some embodiments, the at least one weekly maintenance dose is from 45% to 65% of the loading dose.

In some embodiments, the at least one weekly maintenance dose comprises 20 mg of at least one compound chosen from Compound (I) and pharmaceutically acceptable salts thereof. In some embodiments, the at least one weekly maintenance dose comprises 40 mg of at least one compound chosen from Compound (I) and pharmaceutically acceptable salts thereof. In some embodiments, the at least one weekly maintenance dose comprises 60 mg of at least one compound chosen from Compound (I) and pharmaceutically acceptable salts thereof. In some embodiments, the at least one weekly maintenance dose comprises 80 mg of at least one compound chosen from Compound (I) and pharmaceutically acceptable salts thereof.

In some embodiments, the at least one weekly maintenance dose is effective to achieve a mean Cₘₐₓ at steady state in the patient of at least 400 ng/mL. In some embodiments, the at least one weekly maintenance dose is effective to achieve a mean Cₘₐₓ at steady state in the patient of at least 500 ng/mL. In some embodiments, the at least one weekly maintenance dose is effective to achieve a mean Cₘₐₓ at steady state in the patient of at least 600 ng/mL. In some embodiments, the at least one weekly maintenance dose is effective to achieve a mean Cₘₐₓ at steady state in the patient of at least 700 ng/mL. In some embodiments, the at least one weekly maintenance dose is effective to achieve a mean Cₘₐₓ at steady state in the patient of at least 800 ng/mL. In some embodiments, the at least one weekly maintenance dose is effective to achieve a mean Cₘₐₓ at steady state in the patient of at least 900 ng/mL. In some embodiments, the at least one weekly maintenance dose is effective to achieve a mean Cₘₐₓ at steady state in the patient of at least 1000 ng/mL. In some embodiments, the at least one weekly maintenance dose is effective to achieve a mean Cₘₐₓ at steady state in the patient of at least 1100 ng/mL. In some embodiments, the at least one weekly maintenance dose is effective to achieve a mean Cₘₐₓ at steady state in the patient of at least 1200 ng/mL. In some embodiments, the at least one weekly maintenance dose is effective to achieve a mean Cₘₐₓ at steady state in the patient of at least 1300 ng/mL. In some embodiments, the at least one weekly maintenance dose is effective to achieve a mean Cₘₐₓ at steady state in the patient of at least 1400 ng/mL. In some embodiments, the at least one weekly maintenance dose is effective to achieve a mean Cₘₐₓ at steady state in the patient of at least 1500 ng/mL. In some embodiments, the at least one weekly maintenance dose is effective to achieve a mean Cₘₐₓ at steady state in the patient of at least 1600 ng/mL. In some embodiments, the at least one weekly maintenance dose is effective to achieve a mean Cₘₐₓ at steady state in the patient of at least 1700 ng/mL. In some embodiments, the at least one weekly maintenance dose is effective to achieve a mean Cₘₐₓ at steady state in the patient of at least 1800 ng/mL. In some embodiments, the at least one weekly maintenance dose is effective to achieve a mean Cₘₐₓ at steady state in the patient of at least 1900 ng/mL. In some embodiments, the at least one weekly maintenance dose is effective to achieve a mean Cₘₐₓ at steady state in the patient of at least 2000 ng/mL. In some embodiments, the at least one weekly maintenance dose is effective to achieve a mean Cₘₐₓ at steady state in the patient of at least 2100 ng/mL. In some embodiments, the at least one weekly maintenance dose is effective to achieve a mean Cₘₐₓ at steady state in the patient of at least 2200 ng/mL. In some embodiments, the at least one weekly maintenance dose is effective to achieve a mean Cₘₐₓ at steady state in the patient of at least 2300 ng/mL. In some embodiments, the at least one weekly maintenance dose is effective to achieve a mean Cₘₐₓ at steady state in the patient of at least 2400 ng/mL. In some embodiments, the at least one weekly maintenance dose is effective to achieve a mean Cₘₐₓ at steady state in the patient of at least 2500 ng/mL. In some embodiments, the at least one weekly maintenance dose is effective to achieve a mean Cₘₐₓ at steady state in the patient of at least 2600 ng/mL. In some embodiments, the at least one weekly maintenance dose is effective to achieve a mean Cₘₐₓ at steady state in the patient of at least 2700 ng/mL. In some embodiments, the at least one weekly maintenance dose is effective to achieve a mean Cₘₐₓ at steady state in the patient of at least 2800 ng/mL. In some embodiments, the at least one weekly maintenance dose is effective to achieve a mean Cₘₐₓ at steady state in the patient of at least 2900 ng/mL.

In some embodiments, the at least one weekly maintenance dose is effective to achieve a mean Cₘₐₓ at steady state in the patient of not more than 500 ng/mL. In some embodiments, the at least one weekly maintenance dose is effective to achieve a mean Cₘₐₓ at steady state in the patient of not more than 600 ng/mL. In some embodiments, the at least one weekly maintenance dose is effective to achieve a mean Cₘₐₓ at steady state in the patient of not more than 700 ng/mL. In some embodiments, the at least one weekly maintenance dose is effective to achieve a mean Cₘₐₓ at steady state in the patient of not more than 800 ng/mL. In some embodiments, the at least one weekly maintenance dose is effective to achieve a mean Cₘₐₓ at steady state in the patient of not more than 900 ng/mL. In some embodiments, the at least one weekly maintenance dose is effective to achieve a mean Cₘₐₓ at steady state in the patient of not more than 1000 ng/mL. In some embodiments, the at least one weekly maintenance dose is effective to achieve a mean Cₘₐₓ at steady state in the patient of not more than 1100 ng/mL. In some embodiments, the at least one weekly maintenance dose is effective to achieve a mean Cₘₐₓ at steady state in the patient of not more than 1200 ng/mL. In some embodiments, the at least one weekly maintenance dose is effective to achieve a mean Cₘₐₓ at steady state in the patient of not more than 1300 ng/mL. In some embodiments, the at least one weekly maintenance dose is effective to achieve a mean Cₘₐₓ at steady state in the patient of not more than 1400 ng/mL. In some embodiments, the at least one weekly maintenance dose is effective to achieve a mean Cₘₐₓ at steady state in the patient of not more than 1500 ng/mL. In some embodiments, the at least one weekly maintenance dose is effective to achieve a mean Cₘₐₓ at steady state in the patient of not more than 1600 ng/mL. In some embodiments, the at least one weekly maintenance dose is effective to achieve a mean Cₘₐₓ at steady state in the patient of not more than 1700 ng/mL. In some embodiments, the at least one weekly maintenance dose is effective to achieve a mean Cₘₐₓ at steady state in the patient of not more than 1800 ng/mL. In some embodiments, the at least one weekly maintenance dose is effective to achieve a mean Cₘₐₓ at steady state in the patient of not more than 1900 ng/mL. In some embodiments, the at least one weekly maintenance dose is effective to achieve a mean Cₘₐₓ at steady state in the patient of not more than 2000 ng/mL. In some embodiments, the at least one weekly maintenance dose is effective to achieve a mean Cₘₐₓ at steady state in the patient of not more than 2100 ng/mL. In some embodiments, the at least one weekly maintenance dose is effective to achieve a mean Cₘₐₓ at steady state in the patient of not more than 2200 ng/mL. In some embodiments, the at least one weekly maintenance dose is effective to achieve a mean Cₘₐₓ at steady state in the patient of not more than 2300 ng/mL. In some embodiments, the at least one weekly maintenance dose is effective to achieve a mean Cₘₐₓ at steady state in the patient of not more than 2400 ng/mL. In some embodiments, the at least one weekly maintenance dose is effective to achieve a mean Cₘₐₓ at steady state in the patient of not more than 2500 ng/mL. In some embodiments, the at least one weekly maintenance dose is effective to achieve a mean Cₘₐₓ at steady state in the patient of not more than 2600 ng/mL. In some embodiments, the at least one weekly maintenance dose is effective to achieve a mean Cₘₐₓ at steady state in the patient of not more than 2700 ng/mL. In some embodiments, the at least one weekly maintenance dose is effective to achieve a mean Cₘₐₓ at steady state in the patient of not more than 2800 ng/mL. In some embodiments, the at least one weekly maintenance dose is effective to achieve a mean Cₘₐₓ at steady state in the patient of not more than 2900 ng/mL. In some embodiments, the at least one weekly maintenance dose is effective to achieve a mean Cₘₐₓ at steady state in the patient of not more than 3000 ng/mL.

In some embodiments, the at least one weekly maintenance dose is effective to achieve a mean Cₘₐₓ at steady state in the patient from 400 ng/mL to 3000 ng/mL.

In some embodiments, the at least one weekly maintenance dose is effective to achieve a mean Cₘₐₓ at steady state in the patient from 50% to 150% of 1885 ng/mL. In some embodiments, the at least one weekly maintenance dose is effective to achieve a mean Cₘₐₓ at steady state in the patient from 55% to 145% of 1885 ng/mL. In some embodiments, the at least one weekly maintenance dose is effective to achieve a mean Cₘₐₓ at steady state in the patient from 60% to 140% of 1885 ng/mL. In some embodiments, the at least one weekly maintenance dose is effective to achieve a mean Cₘₐₓ at steady state in the patient from 65% to 135% of 1885 ng/mL. In some embodiments, the at least one weekly maintenance dose is effective to achieve a mean Cₘₐₓ at steady state in the patient from 70% to 130% of 1885 ng/mL. In some embodiments, the at least one weekly maintenance dose is effective to achieve a mean Cₘₐₓ at steady state in the patient from 75% to 125% of 1885 ng/mL. In some embodiments, the at least one weekly maintenance dose is effective to achieve a mean Cₘₐₓ at steady state in the patient from 80% to 120% of 1885 ng/mL. In some embodiments, the at least one weekly maintenance dose is effective to achieve a mean Cₘₐₓ at steady state in the patient from 85% to 115% of 1885 ng/mL. In some embodiments, the at least one weekly maintenance dose is effective to achieve a mean Cₘₐₓ at steady state in the patient from 90% to 110% of 1885 ng/mL. In some embodiments, the at least one weekly maintenance dose is effective to achieve a mean Cₘₐₓ at steady state in the patient from 95% to 105% of 1885 ng/mL.

In some embodiments, the at least one weekly maintenance dose is effective to achieve a mean C_{av,ss} in the patient of at least 200 ng/mL. In some embodiments, the at least one weekly maintenance dose is effective to achieve a mean C_{av,ss} in the patient of not more than 2500 ng/mL. In some embodiments, the at least one weekly maintenance dose is effective to achieve a mean C_{av,ss} in the patient from 200 ng/mL to 2500 ng/mL.

In some embodiments, the at least one weekly maintenance dose is effective to achieve a mean C_{av,ss} in the patient from 50% to 150% of 1353 ng/mL. In some embodiments, the at least one weekly maintenance dose is effective to achieve a mean C_{av,ss} in the patient from 55% to 145% of 1353 ng/mL. In some embodiments, the at least one weekly maintenance dose is effective to achieve a mean C_{av,ss} in the patient from 60% to 140% of 1353 ng/mL. In some embodiments, the at least one weekly maintenance dose is effective to achieve a mean C_{av,ss} in the patient from 65% to 135% of 1353 ng/mL. In some embodiments, the at least one weekly maintenance dose is effective to achieve a mean C_{av,ss} in the patient from 70% to 130% of 1353 ng/mL. In some embodiments, the at least one weekly maintenance dose is effective to achieve a mean C_{av,ss} in the patient from 75% to 125% of 1353 ng/mL. In some embodiments, the at least one weekly maintenance dose is effective to achieve a mean C_{av,ss} in the patient from 80% to 120% of 1353 ng/mL. In some embodiments, the at least one weekly maintenance dose is effective to achieve a mean C_{av,ss} in the patient from 85% to 115% of 1353 ng/mL. In some embodiments, the at least one weekly maintenance dose is effective to achieve a mean C_{av,ss} in the patient from 90% to 110% of 1353 ng/mL. In some embodiments, the at least one weekly maintenance dose is effective to achieve a mean C_{av,ss} in the patient from 95% to 105% of 1353 ng/mL.

In some embodiments, the mean AUC_{τ} at steady state in the patient is not more than 400,000 ng·h/mL. In some embodiments, the mean AUC_{τ} at steady state in the patient is not more than 300,000 ng·h/mL. In some embodiments, the mean AUC_{τ} at steady state in the patient is from 30,000 ng·h/mL to 400,000 ng·h/mL.

In some embodiments, the mean AUC_{τ} at steady state in the patient is from 50% to 150% of 227,230 ng·h/mL. In some embodiments, the mean AUC_{τ} at steady state in the patient is from 55% to 145% of 227,230 ng·h/mL. In some embodiments, the mean AUC_{τ} at steady state in the patient is from 60% to 140% of 227,230 ng·h/mL. In some embodiments, the mean AUC_{τ} at steady state in the patient is from 65% to 135% of 227,230 ng·h/mL. In some embodiments, the mean AUC_{τ} at steady state in the patient is from 70% to 130% of 227,230 ng·h/mL. In some embodiments, the mean AUC_{τ} at steady state in the patient is from 75% to 125% of 227,230 ng·h/mL. In some embodiments, the mean AUC_{τ} at steady state in the patient is from 80% to 120% of 227,230 ng·h/mL. In some embodiments, the mean AUC_{τ} at steady state in the patient is from 85% to 115% of 227,230 ng·h/mL. In some embodiments, the mean AUC_{τ} at steady state in the patient is from 90% to 110% of 227,230 ng·h/mL. In some embodiments, the mean AUC_{τ} at steady state in the patient is from 95% to 105% of 227,230 ng·h/mL.

In some embodiments, the mean t_{1/2z} at steady state in the patient is from 50 h to 500 h. In some embodiments, the mean t_{1/2z} in the patient is from 50 h to 450 h. In some embodiments, the mean t_{1/2z} at steady state in the patient is from 50 h to 400 h. In some embodiments, the mean t_{1/2z} in the patient is from 50 h to 350 h.

In some embodiments, the mean t_{1/2z} at steady state in the patient is from 100 h to 500 h. In some embodiments, the mean t_{1/2z} at steady state in the patient is from 150 h to 500 h. In some embodiments, the mean t_{1/2z} at steady state in the patient is from 200 h to 500 h.

In some embodiments, the mean t_{1/2z} at steady state in the patient is from 150 h to 350 h.

In some embodiments, the mean t_{1/2z} at steady state in the patient is at least 100 h. In some embodiments, the mean t_{1/2z} at steady state in the patient is at least 125 h. In some embodiments, the mean t_{1/2z} at steady state in the patient is at least 150 h. In some embodiments, the mean t_{1/2z} at steady state in the patient is at least 175 h. In some embodiments, the mean t_{1/2z} at steady state in the patient is at least 200 h. In some embodiments, the mean t_{1/2z} at steady state in the patient is at least 225 h. In some embodiments, the mean t_{1/2z} at steady state in the patient is at least 250 h.

In some embodiments, the loading dose comprises 40 mg of at least one compound chosen from Compound (I) and pharmaceutically acceptable salts thereof, and the at least one weekly maintenance dose comprises 20 mg of at least one compound chosen from Compound (I) and pharmaceutically acceptable salts thereof. In some embodiments, the loading dose comprises 80 mg of at least one compound chosen from Compound (I) and pharmaceutically acceptable salts thereof, and the at least one weekly maintenance dose comprises 40 mg of at least one compound chosen from Compound (I) and pharmaceutically acceptable salts thereof. In some embodiments, the loading dose comprises 120 mg of at least one compound chosen from Compound (I) and pharmaceutically acceptable salts thereof, and the at least one weekly maintenance dose comprises 60 mg of at least one compound chosen from Compound (I) and pharmaceutically acceptable salts thereof. In some embodiments, the loading dose comprises 160 mg of at least one compound chosen from Compound (I) and pharmaceutically acceptable salts thereof, and the at least one weekly maintenance dose comprises 80 mg of at least one compound chosen from Compound (I) and pharmaceutically acceptable salts thereof.

In some embodiments, the loading dose is orally administered.

In some embodiments, the loading dose is administered in at least one (e.g., 1, 2, 3, or 4) tablet. In some embodiments, the loading dose is administered in at least one (e.g., 1, 2, 3, or 4) immediate release tablet. In some embodiments, the loading dose is administered in at least one (e.g., 1, 2, 3, or 4) immediate release tablet further comprising mannitol and microcrystalline cellulose.

In some embodiments, the loading dose is administered in an oral suspension. In some embodiments, the loading dose is administered in an oral suspension further comprising Tween and methylcellulose.

In some embodiments, the loading dose is administered with water.

In some embodiments, the loading dose is administered without food. In some embodiments, the patient is in a fasted state. In some embodiments, the patient has fasted for at least 10 h prior to the administration of the loading dose.

In some embodiments, the loading dose is administered with food. In some embodiments, the loading dose is administered with a high-fat, high-calorie meal. In some embodiments, the patient is in a fed state. In some embodiments, the patient ate at least 30 min prior to the administration of the loading dose.

In some embodiments, the at least one weekly maintenance dose is orally administered.

In some embodiments, the at least one weekly maintenance dose is administered in at least one (e.g., 1, 2, 3, or 4) tablet. In some embodiments, the at least one weekly maintenance dose is administered in at least one (e.g., 1, 2, 3, or 4) immediate release tablet. In some embodiments, the at least one weekly maintenance dose is administered in at least one (e.g., 1, 2, 3, or 4) immediate release tablet further comprising mannitol and microcrystalline cellulose.

In some embodiments, the at least one weekly maintenance dose is administered in an oral suspension. In some embodiments, the at least one weekly maintenance dose is administered in an oral suspension further comprising Tween and methylcellulose.

In some embodiments, the at least one weekly maintenance dose is administered without food. In some embodiments, the patient is in a fasted state. In some embodiments, the patient has fasted for at least 10 h prior to the administration of the at least one weekly maintenance dose.

In some embodiments, the at least one weekly maintenance dose is administered with water. In some embodiments, the at least one weekly maintenance dose is administered with food. In some embodiments, the at least one weekly maintenance dose is administered with a high-fat, high-calorie meal. In some embodiments, the patient is in a fed state. In some embodiments, the patient ate at least 30 min prior to the administration of the at least one weekly maintenance dose.

In some embodiments, the methods further comprise administering at least one additional active pharmaceutical ingredient. In some embodiments, the at least one additional active pharmaceutical ingredient is chosen from sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, mGlu2/3 agonists, 5HT-2 antagonists, PDE10 antagonists, and GlyT1 inhibitors.

In some embodiments, the at least one additional active pharmaceutical ingredient is chosen from antipsychotics.

In some embodiments, the at least one additional active pharmaceutical ingredient is chosen from first-generation antipsychotics. In some embodiments, the at least one additional active pharmaceutical ingredient is chosen from chlorpromazine, fluphenazine, haloperidol, and perphenazine.

In some embodiments, the at least one additional active pharmaceutical ingredient is chosen from second-generation antipsychotics. In some embodiments, the at least one additional active pharmaceutical ingredient is chosen from aripiprazole, asenapine, brexpiprazole, cariprazine, clozapine, iloperidone, lurasidone, olanzapine, paliperidone, quetiapine, risperidone, and ziprasidone.

In some embodiments, the at least one additional active pharmaceutical ingredient is chosen from long-acting injectable antipsychotics. In some embodiments, the at least one additional active pharmaceutical ingredient is chosen from aripiprazole, fluphenazine decanoate, haloperidol decanoate, paliperidone, and risperidone.

In some embodiments, the methods comprise treating at least one negative symptom of schizophrenia. In some embodiments, the at least one negative symptom of schizophrenia is chosen from anhedonia, loss of motivation, and reduced interest in social interaction. In some embodiments, the at least one negative symptom of schizophrenia is anhedonia. In some embodiments, the at least one negative symptom of schizophrenia is loss of motivation. In some embodiments, the at least one negative symptom of schizophrenia is reduced interest in social interaction.

In some embodiments, the methods comprise treating at least one cognitive symptom of schizophrenia. In some embodiments, the at least one cognitive symptom of schizophrenia is chosen from impaired verbal memory, impaired working memory, impaired motor function, impaired attention and processing speed, impaired verbal fluency, and impaired executive function. In some embodiments, the at least one cognitive symptom of schizophrenia is impaired verbal memory. In some embodiments, the at least one cognitive symptom of schizophrenia is impaired working memory. In some embodiments, the at least one cognitive symptom of schizophrenia is impaired motor function. In some embodiments, the at least one cognitive symptom of schizophrenia is impaired attention and processing speed. In some embodiments, the at least one cognitive symptom of schizophrenia is impaired verbal fluency. In some embodiments, the at least one cognitive symptom of schizophrenia is impaired executive function.

In some embodiments, the methods comprise modulating reward anticipation related brain activity in the patient.

In some embodiments, the methods comprise modulating cerebral blood flow in the patient.

In some embodiments, the methods comprise increasing ventral striatal activity during reward anticipation in the patient.

In some embodiments, the methods comprise modulating dopamine release in the patient. In some embodiments, the methods comprise reducing dopamine release in the patient. In some embodiments, the methods comprise reducing dopamine release in the patient following an exposure to a stimulant.

### Pharmaceutical Compositions:

Some embodiments of the present disclosure relate to pharmaceutical compositions comprising: at least one compound chosen from Compound (I) and pharmaceutically acceptable salts thereof, and at least one pharmaceutically acceptable excipient. In some embodiments, the pharmaceutical compositions comprise more than 80 mg of at least one compound chosen from Compound (I) and pharmaceutically acceptable salts thereof. In some embodiments, the pharmaceutical compositions comprise more than 100 mg of at least one compound chosen from Compound (I) and pharmaceutically acceptable salts thereof. In some embodiments, the pharmaceutical compositions comprise 120 mg of at least one compound chosen from Compound (I) and pharmaceutically acceptable salts thereof. In some embodiments, the pharmaceutical compositions comprise 160 mg of at least one compound chosen from Compound (I) and pharmaceutically acceptable salts thereof.

In some embodiments, the pharmaceutical composition is in the form of at least one (e.g., 1, 2, 3, or 4) tablet. In some embodiments, the pharmaceutical composition is in the form of at least one (e.g., 1, 2, 3, or 4) immediate release tablet. In some embodiments, the pharmaceutical composition is in the form of at least one (e.g., 1, 2, 3, or 4) immediate release tablet comprising mannitol and microcrystalline cellulose.

In some embodiments, the pharmaceutical composition is in the form of at least one tablet comprising: a core composition comprising at least one compound chosen from Compound (I) and pharmaceutically acceptable salts thereof, and a coating layer covering the core composition.

In some embodiments, the core composition further comprises mannitol. In some embodiments, the core composition further comprises microcrystalline cellulose. In some embodiments, the core composition further comprises hydroxypropyl cellulose. In some embodiments, the core composition further comprises croscarmellose sodium.

In some embodiments, the core composition further comprises mannitol and microcrystalline cellulose. In some embodiments, the core composition further comprises mannitol and hydroxypropyl cellulose. In some embodiments, the core composition further comprises mannitol and croscarmellose sodium.

In some embodiments, the core composition further comprises mannitol, microcrystalline cellulose, and hydroxypropyl cellulose. In some embodiments, the core composition further comprises mannitol, microcrystalline cellulose, and croscarmellose sodium.

In some embodiments, the core composition further comprises mannitol, microcrystalline cellulose, hydroxypropyl cellulose, and croscarmellose sodium.

In some embodiments, the core composition comprises 5% to 25% of at least one compound chosen from Compound (I) and pharmaceutically acceptable salts thereof by weight of the core composition. In some embodiments, the core composition comprises 7.5% to 22.5% of at least one compound chosen from Compound (I) and pharmaceutically acceptable salts thereof by weight of the core composition. In some embodiments, the core composition comprises 10% to 20% of at least one compound chosen from Compound (I) and pharmaceutically acceptable salts thereof by weight of the core composition.

In some embodiments, the core composition further comprises 55% to 75% mannitol by weight of the core composition. In some embodiments, the core composition further comprises 60% to 75% mannitol by weight of the core composition. In some embodiments, the core composition further comprises 65% to 75% mannitol by weight of the core composition. In some embodiments, the core composition further comprises 60% to 70% mannitol by weight of the core composition.

In some embodiments, the core composition further comprises 5% to 15% microcrystalline cellulose by weight of the core composition. In some embodiments, the core composition further comprises 7.5% to 12.5% microcrystalline cellulose by weight of the core composition. In some embodiments, the core composition further comprises 10% microcrystalline cellulose by weight of the core composition.

In some embodiments, the core composition further comprises 2.5% to 7.5% croscarmellose sodium by weight of the core composition. In some embodiments, the core composition further comprises 5% croscarmellose sodium by weight of the core composition.

In some embodiments, the core composition further comprises 1% to 5% hydroxypropyl cellulose by weight of the core composition. In some embodiments, the core composition further comprises 3% hydroxypropyl cellulose by weight of the core composition.

In some embodiments, the core composition further comprises 0.5% to 1.5% magnesium stearate by weight of the core composition. In some embodiments, the core composition further comprises 1% magnesium stearate by weight of the core composition.

In some embodiments, the core composition comprises, by weight of the core composition:
5% to 25% of at least one compound chosen from Compound (I) and pharmaceutically acceptable salts thereof;
55% to 75% mannitol; and
5% to 15% microcrystalline cellulose.

In some embodiments, the core composition comprises, by weight of the core composition:
10% to 20% of at least one compound chosen from Compound (I) and pharmaceutically acceptable salts thereof;
60% to 75% mannitol; and
7.5% to 12.5% microcrystalline cellulose.

In some embodiments, the pharmaceutical composition is in the form of one tablet. In some embodiments, the pharmaceutical composition is in the form of two tablets. In some embodiments, the pharmaceutical composition is in the form of three tablets. In some embodiments, the pharmaceutical composition is in the form of four tablets. In some embodiments, the pharmaceutical composition is in the form of five tablets. In some embodiments, the pharmaceutical composition is in the form of six tablets.

In some embodiments, the pharmaceutical composition is in the form of two tablets, each tablet comprising 40 mg of at least one compound chosen from Compound (I) and pharmaceutically acceptable salts thereof.

In some embodiments, the pharmaceutical composition is in the form of four tablets, each tablet comprising 40 mg of at least one compound chosen from Compound (I) and pharmaceutically acceptable salts thereof.

In some embodiments, the pharmaceutical composition is in the form of an oral suspension. In some embodiments, the pharmaceutical composition is in the form of an oral suspension comprising Tween and methylcellulose.

The proportion and nature of any pharmaceutically acceptable excipient may be determined by the chosen route of administration and standard pharmaceutical practice. Except insofar as any conventional pharmaceutically acceptable excipient is incompatible with Compound (I), such as by producing any undesirable biological effect or otherwise interacting in a deleterious manner with any other component(s) of the pharmaceutically composition, its use is contemplated to be within the scope of this disclosure.

Some non-limiting examples of materials which may serve as pharmaceutically acceptable excipients include: (1) sugars, such as, e.g., lactose, glucose, and sucrose; (2) starches, such as, e.g., corn starch and potato starch; (3) cellulose and its derivatives, such as, e.g., sodium carboxymethyl cellulose, ethyl cellulose, and cellulose acetate; (4) powdered tragacanth; (5) malt; (6) gelatin; (7) talc; (8) excipients, such as, e.g., cocoa butter and suppository waxes; (9) oils, such as, e.g., peanut oil, cottonseed oil, safflower oil, sesame oil, olive oil, corn oil, and soybean oil; (10) glycols, such as, e.g., propylene glycol; (11) polyols, such as, e.g., glycerin, sorbitol, mannitol, and polyethylene glycol; (12) esters, such as, e.g., ethyl oleate and ethyl laurate; (13) agar; (14) buffering agents, such as, e.g., magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York also discloses additional non-limiting examples of pharmaceutically acceptable excipients, as well as known techniques for preparing and using the same.

In effecting treatment of a patient in need thereof, at least one compound chosen from Compound (I) and pharmaceutically acceptable salts thereof can be administered in any form and route which makes the API bioavailable. Illustratively, in some embodiments, at least one compound chosen from Compound (I) and pharmaceutically acceptable salts thereof may be administered by a variety of routes, such as, e.g., oral administration (such as, e.g., via tablets or capsules). In some embodiments, when aqueous suspensions are employed for oral administration, at least one compound chosen from Compound (I) and pharmaceutically acceptable salts thereof may be combined with emulsifying and suspending agents.

In some embodiments, at least one compound chosen from Compound (I) and pharmaceutically acceptable salts thereof may be administered by a parenteral route, such as, e.g., by inhalation, subcutaneously, intramuscularly, intravenously, intraarterially, transdermally, intranasally, rectally, vaginally, ocularly, topically, sublingually, buccally, intraperitoneally, intraadiposally, intrathecally, or via local delivery (such as, e.g., by catheter or stent). In some embodiments, sterile injectable forms of the pharmaceutical compositions of this disclosure may be aqueous or oleaginous suspensions. These suspensions may be formulated according to techniques known in the art using suitable dispersing or wetting agents and suspending agents. In some embodiments, the sterile injectable preparation may also be a sterile injectable solution or suspension in a non-toxic parenterally acceptable diluent or solvent, such as, e.g., a solution in 1,3-butanediol. Non-limiting examples of acceptable vehicles and solvents that may be employed are water, Ringer's solution, and isotonic sodium chloride solution. In additional non-limiting examples, sterile, fixed oils may be employed as a solvent or suspending medium.

One skilled in the art can readily select the proper form and route of administration depending upon the disorder or condition to be treated, the stage of the disorder or condition, and other relevant circumstances. In some embodiments, pharmaceutical compositions of the disclosure may be administered to the patient in the form of tablets, capsules, cachets, papers, lozenges, wafers, elixirs, ointments, transdermal patches, aerosols, inhalants, suppositories, solutions, or suspensions.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Furthermore, the disclosure encompasses all variations, combinations, and permutations in which at least one limitation, element, clause, and descriptive term from at least one of the listed claims is introduced into another claim. For example, any claim that is dependent on another claim can be modified to include at least one limitation found in any other claim that is dependent on the same base claim. Where elements are presented as lists, such as, e.g., in Markush group format, each subgroup of the elements is also disclosed, and any element(s) can be removed from the group. It should be understood that, in general, where the disclosure, or aspects of the disclosure, is/are referred to as comprising particular elements and/or features, embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those of ordinary skill in the art will recognize or be able to ascertain using no more than routine experimentation, many equivalents to the specific embodiments of the disclosure described herein. Such equivalents are intended to be encompassed by the following claims.

### EXAMPLES

The following examples are intended to be illustrative and are not meant in any way to limit the scope of the disclosure.

### ABBREVIATIONS

% Percent
% CV Percent coefficient of variance
AE Adverse event
ANOVA ANalysis of Variance
ASL Arterial spin labeling
AUC Area under the plasma concentration-time curve
AUC₂₄ AUC from time 0 to 24 h
AUC₉₆ AUC from time 0 to 96 h
AUC_{τ} AUC during the dosing interval
BACS Brief Assessment of Cognition in Schizophrenia
BOLD Blood oxygenation level dependent
BMI Body mass index
BNSS Brief Negative Symptom Scale
C_{av,ss} Average plasma concentration at steady state
Cₘₐₓ Maximum observed plasma concentration
C_{trough} Observed plasma concentration at the end of a dosing interval
Ca Caudate nucleus
CBF Cerebral blood flow
CL/F Apparent clearance after extravascular administration
CSSRS Columbia Suicide Severity Rating Scale
CYP3A4 Cytochrome P450 3A4
d-APMH Dextroamphetamine
DSM Diagnostic and Statistical Manual of Mental Disorders
FIH First-in-human
fMRI Functional magnetic resonance imaging
g Gram
GAD Generalized Anxiety Disorder
GCP Good Clinical Practice
GP Globus pallidus
h Hour(s)
IA Interim Analysis
ICH International Conference on Harmonization
IRB Institutional Review Board
kg Kilogram(s)
m² Square meter(s)
MC Methylcellulose
mg Milligram
MID Monetary Incentive Delay
min Minute(s)
MINI Mini International Neuropsychiatric Interview
mL Milliliter(s)
mm Millimeter(s)
MNI Montreal Neurological Institute
MP-RAGE Magnetization-prepared rapid gradient-echo
MRD Multiple-rising dose
MR Magnetic resonance
MRI Magnetic resonance imaging
NAcc Nucleus accumbens
NSFS Negative symptoms factor score
PANSS Positive and Negative Syndrome Scale
pCASL Pseudo-continuous ASL
PET Positron emission tomography
PK Pharmacokinetics
PD Pharmacodynamics
Pu Putamen
R_{ac(AUC)} Accumulation ratio (based on AUC_{τ})
R_{ac(Cmax)} Accumulation ratio (based on Cₘₐₓ)
RBA Relative bioavailability
RF Radiofrequency
ROI Region of interest
s Second(s)
SAP Statistical analysis plan
SD Standard deviation
SE Standard error
SN Substantia nigra
SPECT Single-photon emission computed tomography
SPM Statistical parametric mapping
SRD Single-rising dose
SRTM Simplified reference tissue model
t_{1/2z} Terminal disposition phase half-life
tₘₐₓ Time of first occurrence of Cₘₐₓ
TAC Time-activity curve
TEAE Treatment-emergent adverse event
V_{z}/F Apparent volume of distribution during the terminal disposition phase after extravascular administration
VSt Ventral striatum (VSt)
VTA Ventral tegmental area

### Example 1: Phase 1 First-In-Human Safety, Tolerability, and Pharmacokinetics Study for Compound (I)

A phase 1 first-in-human (FIH) study was conducted at a single site in the United States to evaluate the safety, tolerability, and pharmacokinetics (PK) of Compound (I). Specifically, Compound (I) was tested to evaluate the safety, tolerability, and PK of single and multiple doses in healthy participants and as add-on therapy to antipsychotics in participants with stable schizophrenia. The phase I study also assessed the oral bioavailability of Compound (I) in healthy participants administered a tablet formulation compared to an oral suspension formulation in a fasted state, as well the effect of a high-fat, high-calorie meal on the PK of a single dose of a tablet formulation of Compound (I). Additional exploratory pharmacodynamic analysis was undertaken in patients with stable schizophrenia to inform future studies.

In the phase 1 study, Compound (I) was rapidly absorbed following single and repeated oral administration in both healthy volunteers and patients with stable schizophrenia. Following single and repeated oral Compound (I) administration, the urine 6β-hydroxycortisol/cortisol ratio was not meaningfully different to placebo, suggesting CYP3A4 was not induced. Compound (I) was generally well-tolerated, with no severe or serious adverse safety signals reported.

Study Design

The study complied with the IRB regulations stated in GCP regulations and guidelines and all applicable local regulations. The study was conducted with the highest respect for the individual participants and in accordance with the ethical principles that have their origin in the Declaration of Helsinki, the requirements and definitions of ICH Harmonized Tripartite Guideline for GCP, and all applicable local regulations. All participants signed and dated informed consent forms before any protocol-specific screening procedures.

As shown in Table 1, the study was composed of 4 parts: (1) Part 1 (single-rising dose [SRD], alternating panel design and a sequential panel design); (2) Part 2 (multiple-rising dose [MRD], sequential panel design); (3) Part 3 (open label parallel design); and (4) Part 4 (single dose cohort). Cohorts in Parts 1 and 2 were randomized to receive either Compound (I) or placebo in a 6:2 ratio. Cohorts in Part 3 were randomized in a 1:1 ratio, while the cohort in Part 4 was randomized in a 2:1 ratio.

TABLE 1
Phase 1 FIH Study Arms

| Arms | Assigned Interventions |
|---|---|
| Placebo Comparator: Part 1 (SRD): Placebo Cohorts | Drug: Compound (I) Placebo |
| 1-5 | (Suspension) |
| Compound (I) placebo-matching suspension, orally, | Compound (I) placebo-matching |
| once on Day 1 in fasted healthy participants in the | suspension. |
| SRD period. | |
| Experimental: Part 1 (SRD): Cohort 1: Compound (I) | Drug: Compound (I) Suspension |
| 5/20 mg | Compound (I) oral suspension. |
| Compound (I) 5 mg, suspension, orally, once on Day | |
| 1 in fasted healthy participants in the SRD period. | |
| Participants also received 20 mg, suspension, orally, | |
| once on Day 8 in the SRD period. | |
| Experimental: Part 1 (SRD): Cohort 2: Compound (I) | Drug: Compound (I) Suspension |
| 10/40 mg | Compound (I) oral suspension. |
| Compound (I) 10 mg, suspension, orally, once on | |
| Day 1 in fasted healthy participants in the SRD | |
| period. Participants also received 40 mg, suspension, | |
| orally, once on Day 8 in the SRD period. | |
| Experimental: Part 1 (SRD): Cohort 3: Compound (I) | Drug: Compound (I) Suspension |
| 80 mg | Compound (I) oral suspension. |
| Compound (I) 80 mg, suspension, orally, once on | |
| Day 1 in fasted healthy participants in the SRD | |
| period. | |
| Experimental: Part 1 (SRD): Cohort 4: Compound (I) | Drug: Compound (I) Suspension |
| 120 mg | Compound (I) oral suspension. |
| Compound (I) 120 mg, suspension, orally, once on | |
| Day 1 in fasted healthy participants in the SRD | |
| period. | |
| Experimental: Part 1 (SRD): Cohort 5: Compound (I) | Drug: Compound (I) Suspension |
| 160 mg | Compound (I) oral suspension. |
| Compound (I) 160 mg, suspension, orally, once on | |
| Day 1 in fasted healthy participants in the SRD | |
| period. | |
| Placebo Comparator: Part 2 (MRD): Placebo Cohorts | Drug: Compound (I) Placebo |
| 1-4 | (Suspension) |
| Compound (I) placebo-matching suspension, orally, | Compound (I) placebo-matching |
| once on Day 1 in fasted healthy participants in the | suspension. |
| MRD period. | |
| Experimental: Part 2 (MRD): Cohort 1: Compound | Drug: Compound (I) Suspension |
| (I) 40/20 mg | Compound (I) oral suspension. |
| Compound (I) 40 mg as loading dose, suspension, | |
| orally, once on Day 1 in fasted healthy participants | |
| followed by 20 mg (half the initial dose) as a | |
| maintenance dose on Days 8, 15 and 22 in the MRD | |
| period. | |
| Experimental: Part 2 (MRD): Cohort 2: Compound | Drug: Compound (I) Suspension |
| (I) 80/40 mg | Compound (I) oral suspension. |
| Compound (I) 80 mg as loading dose, suspension, | |
| orally, once on Day 1 in fasted healthy participants | |
| followed by 40 mg (half the initial dose) as a | |
| maintenance dose on Days 8, 15 and 22 in the MRD | |
| period. | |
| Experimental: Part 2 (MRD): Cohort 3: Compound | Drug: Compound (I) Suspension |
| (I) 120/60 mg | Compound (I) oral suspension. |
| Compound (I) 120 mg as loading dose, suspension, | |
| orally, once on Day 1 in fasted healthy participants | |
| followed by 60 mg (half the initial dose) as a | |
| maintenance dose on Days 8, 15 and 22 in the MRD | |
| period. | |
| Experimental: Part 2 (MRD): Cohort 4: Compound | Drug: Compound (I) Suspension |
| (I) 160/80 mg | Compound (I) oral suspension. |
| Compound (I) 160 mg as loading dose, suspension, | |
| orally, once on Day 1 in fasted healthy participants | |
| followed by 80 mg (half the initial dose) as a | |
| maintenance dose on Days 8, 15 and 22 in the MRD | |
| period. | |
| Placebo Comparator: Part 3: Relative Bioavailability | Drug: Compound (I) Placebo (Tablet) |
| (RBA)/Food Effect: Regimen A | Compound (I) placebo-matching |
| Compound (I) placebo-matching, tablet, orally, once | tablet. |
| on Day 1 in fasted state (Regimen A) in Cohort 1. | |
| Experimental: Part 3: RBA/Food Effect: Regimen B | Drug: Compound (I) Tablet |
| Compound (I) 40 mg, tablet, orally, once on Day 1 in | Compound (I) oral tablet. |
| fed state (Regimen B) in Cohort 2. | |
| Placebo Comparator: Part 4: MRD: Placebo | Drug: Compound (I) Placebo |
| Compound (I) placebo-matching, suspension, orally, | (Suspension) |
| on Days 1, 8, 15 and 22 in participants with | Compound (I) placebo-matching |
| schizophrenia. | suspension. |
| Experimental: Part 4: MRD: Compound (I) 160/80 | Drug: Compound (I) Suspension |
| mg | Compound (I) oral suspension. |
| Compound (I) 160 mg as loading dose, suspension, | |
| orally, once on Day 1 followed by 80 mg (half the | |
| initial dose) as a maintenance dose on Days 8, 15 and | |
| 22 in participants with schizophrenia. | |

Part 1 was a double-blind, SRD study in healthy adults, utilizing an alternate panel design (cohorts 1, 2) and sequential panel design (cohorts 3-5). Cohorts 1 and 2 were part of a two-period, alternating-panel, double-blind study of Compound (I) administered as an oral suspension (5 mg, 10 mg, 20 mg, or 40 mg) or matched placebo, with a washout period of at least 7 days between treatment periods. Cohorts 3-5 were part of a sequential-panel, double-blind study to evaluate the SRDs of Compound (I) (80, 120, or 160 mg, respectively) or matched placebo. Although planned, all subsequent doses after the dose of 80 mg were determined based on emerging safety, tolerability, and PK data from the preceding cohorts.

A sentinel group was used for cohort 1, period 1, where the initial two participants were allocated 1:1 to receive either Compound (I) or placebo to ensure adequate safety and tolerability before dosing the remaining participants in the cohort. The remaining six participants were dosed following a review of 24 h post-dose safety and tolerability data. Subsequent cohorts were dosed based on a minimum of 21 days of emerging safety, tolerability, and available PK data from the previous cohorts.

Part 2 was an MRD study in healthy adults to assess plasma exposure and accumulation of Compound (I), carried out in four cohorts (1-4). Part 2 did not commence until 21 days of safety, tolerability, and available PK data had been collected from cohort 3 in Part 1.

In Part 2, participants received either placebo on all dosing days or a loading dose of Compound (I) on Day 1 and a maintenance dose of half the initial dose on Days 8, 15 and 22, administered as an oral suspension. Baseline measurements were obtained on Day −2, and study-specific measurements were obtained following oral dose administrations on Days 1, 8, 15, and 22.

Participants were required to stay in the study unit for 5 days for each cycle of Compound (I) administration. For the loading dose administration, participants were required to remain in the study unit from Day −2 to Day 3, and for the maintenance doses on Day 7-10, Day 14-17, and Day 21-24. Study-specific measurements were conducted in the 48 h period post-dose before participants were discharged. After discharge on Day 24, participants returned for follow-up on Days 26, 29, 36, 43, 50, 57, and 64. A final visit that completed the study occurred 12 to 16 days following the final safety and PK follow-up.

Part 3 was a randomized, open-label, single-dose, parallel design study to evaluate the bioavailability of a Compound (I) tablet formulation relative to a Compound (I) oral suspension formulation and the effect of food on the PK in healthy participants. The immediate release tablet contained mannitol, microcrystalline cellulose, and other commonly used excipients. Participants were randomized 1:1 to receive a single 40 mg dose of Compound (I) as a tablet after either 10 h overnight fast, or 30 min after beginning ingestion of a high-fat, high-calorie breakfast.

Participants were required to remain in the study unit for an additional 48 h post-dose for safety and PK assessments with follow-up visits on Day 4-5 and on Day 17-21. Blood samples were collected over 96 h post-dose to measure the plasma concentrations of Compound (I).

Part 4 was a double-blind, weekly single dosing, parallel group design in patients with stable schizophrenia. Part 4 did not start until 21 days of safety, tolerability, and PK data had been collected in Part 2.

Participants were randomly assigned 2:1 to receive either Compound (I) or placebo. For the participants receiving Compound (I), a loading dose was administered on Day 1, and a maintenance dose, at half the initial dose, on Days 8, 15, and 22. Participants in the placebo group received placebo on all study dosing days.

Participants were required to stay in the study unit for 5 days for each cycle of Compound (I) administration. For the loading dose administration, participants were required to remain in the study unit from Day −2 to Day 3 and for the maintenance doses Day 7-10, Day 14-17, Day 21-24. Study-specific measurements were conducted in the 48 h period post-dose before participants were discharged. After discharge on Day 24, participants returned for follow-up on Days 26, 29, 36, 43, 50, 57, and 64. A final visit that completed the study occurred 12 to 16 days following the final safety and PK follow-up.

Key Inclusion Criteria

The study enrolled men and women aged 18-55 years at the time of informed consent, with a body mass index (BMI) of 18-32 kg/m² for healthy participants, or 18-40.5 kg/m² for participants with schizophrenia.

In Part 4, participants met schizophrenia criteria as defined by the Diagnostic and Statistical Manual of Mental Disorders, 5th Edition (DSM-5) by the Mini International Neuropsychiatric Interview (MINI), a Positive and Negative Syndrome Scale (PANSS) total score ≤90 and a sum of PANSS N1, N2, N3, N4, N6, G7 and G16≥15 at screening and baseline. In addition, participants had to be on a stable dose of antipsychotic medication for at least two months.

Safety Assessments

Safety measurements used in the phase 1 study included treatment-emergent adverse events (TEAEs), physical examinations, weight, height, BMI, vital signs, clinical laboratory evaluations, pregnancy monitoring, and ECG procedures.

TEAEs were defined as any adverse event that occurred or worsened after receiving the first dose of Compound (I) and within 6 weeks after the last dose of Compound (I). Participants experiencing two or more different TEAEs were counted only once. Frequent TEAEs were defined as adverse events that occurred in at least two participants in any treatment. Participants were evaluated with respect to mood and alertness (BL-VAS) and suicidality (C-SSRS).

Pharmacokinetic Assessments

Plasma and urine concentrations of Compound (I) and urine concentrations of cortisol and 6β-hydroxycortisol were measured by high performance liquid chromatography with tandem mass spectrometry. One 4 mL blood sample was collected per scheduled assessment. For participants receiving placebo, blood samples were analyzed only at pre-dose and around the time Cₘₐₓ occurred (as emerging from the measurements of the first dose group) to ensure that no additional participants could have been on active treatment. Serial urine samples were collected for Part 1 cohorts 1-5 and Part 2 cohorts 1-4. Urine samples for participants receiving placebo were not analyzed. Urine samples were collected for the determination of cortisol and 6β-hydroxycortisol for Part 2 cohorts 1-4 and for all patients in Part 4. Samples for participants randomized to placebo were analyzed for these parameters.

Pharmacokinetic parameters were determined from the concentration-time profiles for all evaluable participants. Actual sampling times were used in all computations. pharmacokinetic parameters were calculated using non-compartmental analysis using Phoenix WinNonLin v8.0 or SAS v 9.4.

Pharmacodynamic Assessments

Exploratory pharmacodynamic assessments in Part 4 included: the Positive and Negative Symptom Scale (PANSS) total, subscale and factor scores; Brief Assessment of Cognition in Schizophrenia (BACS) total score; Clinical Global Impression-Severity scale (CGI-S) and Clinical Global Impression-Global Improvement (CGI); Brief Negative Symptom Scale (BNSS) total score; and temporal Experience of Pleasure Scale (TEPS) total score.

Statistics

In the phase 1 study, three analysis sets were used: (1) the safety set (all participants who received at least one dose of the study drug); (2) the PK set (all participants who received study drug and provided sufficient data for at least a single PK parameter); and (3) the PD set (Part 4 only) (participants who received study drug and provided sufficient data for one baseline and one post-baseline scheduled PD parameter).

The target sample sizes for Part 1 cohorts 1-5 and Part 2 cohorts 1-4 were based on precedents of other FIH studies, rather than a formal assessment of statistical power. A sample size of 8 participants per cohort was considered sufficient for investigating the objectives of the study and characterizing any potential effects on safety parameters.

PK outcomes were summarized using descriptive statistics.

Demographic and Other Baseline Characteristics and Study Inclusion

Demographics and general characteristics were generally similar between placebo and active treatment arms (Table 2).

One participant in the 160/80 mg Compound (I) cohort in Part 4 (MRD study with patients with stable schizophrenia) missed the Day 15 dose. Pharmacokinetic concentrations for Day 22 for this participant were excluded from the mean plasma-concentration time profile and associated pharmacokinetic parameters were excluded from statistical analysis.

One participant in the 160/80 mg Compound (I) cohort in Part 4 (MRD study with patients with stable schizophrenia) terminated the study following dosing on Day 1. The participant's PK parameters were excluded from statistical analysis.

Another participant in the 160/80 mg Compound (I) cohort in Part 4 (MRD study with patients with stable schizophrenia) terminated the study following dosing on Day 22. Accordingly, all PK parameters for the participant other than tₘₐₓ, Cₘₐₓ, observed plasma concentration at the end of a dosing interval, AUC₂₄, AUC₄₈, and AUC₉₆ were excluded from summary and statistical analysis.

TABLE 2
Demographic and Baseline Characteristics

| | Part 1 | | Part 2 | | Part 3 | | Part 4 | |
|---|---|---|---|---|---|---|---|---|
| | n = 40 | | n = 32 | | n = 18 | | n = 24 | |
| Age (years) | | | | | | | | |
| Mean (SD) | 39.1 | (9.58) | 38.5 | (9.82) | 34.8 | (8.71) | 44.8 | (7.33) |
| Median (minimum, maximum) | 22, 54 | | 23, 55 | | 23, 54 | | 28, 55 | |
| Gender (%) | | | | | | | | |
| Male | 30 | (75.0) | 26 | (81.3) | 15 | (83.3) | 19 | (79.2) |
| Female | 10 | (25.0) | 6 | (18.8) | 3 | (16.7) | 5 | (20.8) |
| Race (%) | | | | | | | | |
| Asian | 3 | (7.5) | 4 | (12.5) | 2 | (11.1) | 3 | (12.5) |
| Black or African American | 12 | (30.0) | 7 | (21.9) | 3 | (16.7) | 13 | (54.2) |
| Native Hawaiian or other | 1 | (2.5) | — | | — | | — | |
| Pacific Islander | | | | | | | | |
| White | 20 | (50.0) | 19 | (59.4) | 12 | (66.7) | 8 | (33.3) |

| | | | | | | | |
|---|---|---|---|---|---|---|---|
| Multiracial | 4 | (10.0) | 2 | (6.3) | 1 | (5.6) | 0 |

| | | | | |
|---|---|---|---|---|
| BMI (kg/m²) | | | | |
| Mean (SD) | 26.35 | 26.32 | 25.22 | 31.47 |
| Median (minimum, maximum) | (3.118) | (3.351) | (3.142) | (5.838) |
| | 19.6, 31.5 | 18.9, 31.9 | 19.4, 31.5 | 21.6, 39.6 |

Safety Evaluations

No clinically significant changes in baseline characteristics were observed during the study. Of the 114 randomized participants, all received at least one dose of Compound (I) and were included in the safety set. TEAEs experienced by participants were mild, except for two participants (one in Part 2 and one in Part 3) who experienced TEAEs of moderate intensity. No deaths, serious adverse events, or other significant TEAEs were reported. Headache tended to occur more frequently with Compound (I) than with placebo, and its frequency appeared to increase with higher doses. No discontinuations due to TEAEs occurred.

In Part 1 (SRD study), four participants (40.0%) who received placebo and six participants (20.0%) who received Compound (I) experienced a TEAE during the study. None of the TEAEs were considered to be related to the study drug by the investigator.

In Part 2 (MRD study), three participants (37.5%) who received placebo and 19 participants (59.4%) in total experienced a TEAE during the study (Table 3). One TEAE was moderate, while all the other TEAEs were mild.

In Part 3 (relative bioavailability/food effect study), nine participants who were administered a single dose of Compound (I) after an overnight fast of at least 10 h, one (11.1%) experienced a TEAE. Two participants (22.2%) who had received a single Compound (I) dose following a high-fat, high-calorie breakfast experienced TEAEs. One participant experienced a moderate TEAE, whereas all other TEAEs were mild. No deaths, serious AEs, or other TEAEs were reported.

In Part 4 (MRD study with patients with stable schizophrenia), six participants (75.0%) who received placebo and 15 participants (62.5%) in total experienced a TEAE during the study (Table 4). A similar frequency of TEAEs related to the study drug was reported by patients with schizophrenia who received placebo (50.0%) and Compound (I) (56.3%). All TEAEs were mild.

TABLE 3
Overview of TAEs Following MRD in Healthy Participants

| | Placebo | 40/20 | 80/40 | 120/60 | 160/80 | All |
|---|---|---|---|---|---|---|
| | N = 8 | N = 6 | N = 6 | N = 6 | N = 6 | N = 32 |
| Any TEAE | 3 (37.5) | 4 (66.7) | 4 (66.7) | 3 (50.0) | 5 (83.3) | 19 (59.4) |
| Related | 2 (25.0) | — | 1 (16.7) | — | 3 (50.0) | 6 (18.8) |
| Not related | 1 (12.5) | 4 (66.7) | 3 (50.0) | 3 (50.0) | 2 (33.3) | 13 (40.6) |
| Mild | 3 (37.5) | 4 (66.7) | 4 (66.7) | 2 (33.3) | 5 (83.3) | 18 (56.3) |
| Moderate | — | — | — | 1 (16.7) | — | 1 (3.1) |
| Serious | — | — | — | — | — | — |
| Drug | — | — | — | — | — | — |
| discontinuation | | | | | | |
| Deaths | — | — | — | — | — | — |

TABLE 4
Overview of TAEs Following MRD in
Patients with Stable Schizophrenia

| | Placebo | 160/80 | All |
|---|---|---|---|
| | N = 8 | N = 16 | N = 24 |
| Any TEAE | 6 (75.0) | 9 (56.3) | 15 (62.5) |
| Related | 4 (50.0) | 9 (56.3) | 13 (54.2) |
| Not related | 2 (25.0) | — | 2 (8.3) |
| Mild | 6 (75.0) | 9 (56.3) | 15 (62.5) |
| Moderate | — | — | — |
| Serious | — | — | — |
| Drug | — | — | — |
| discontinuation | | | |
| Deaths | — | — | — |

Compound (I) Pharmacokinetics

Compound (I) was rapidly absorbed following single and repeated oral administration in healthy and schizophrenia patients, with a tₘₐₓ of 0.99-3.00 h. At higher doses (≥120 mg), a secondary peak was sometimes observed. Following single oral doses of Compound (I) in healthy patients, Cₘₐₓ and AUC increased less than dose proportionally with increasing doses (5-160 mg). Mean Cₘₐₓ values for participants in Part 1 (observed up to 96 h post-dose for cohorts 1 and 2 and up to 168 h post-dose for cohorts 3-5) are shown in Table 5. Mean Cₘₐₓ values for participants in some cohorts of Parts 2 and 4 (observed up to 168 h post-dose) are shown in Table 6.

TABLE 5
Mean Cₘₐₓ Values for Healthy Participants in Part 1 (SRD Study)

| | Cohort 1: | Cohort 2: | Cohort 3: | Cohort 4: | Cohort 5: |
|---|---|---|---|---|---|
| | Compound | Compound | Compound | Compound | Compound |
| | (I) | (I) | (I) | (I) | (I) |
| | 5 mg | 10 mg | 80 mg | 120 mg | 160 mg |
| N | 6 | 6 | 6 | 6 | 6 |
| Mean (SD) | 139.8 (15.25) | 193.3 (27.75) | 909.5 (225.29) | 1200.0 (147.38) | 1121.7 (175.31) |
| [ng/mL] | | | | | |

TABLE 6
Mean Cₘₐₓ Values for Participants in MRD Studies

| | Part 2: | Part 2: | Part 2: | Part 2: | Part 4: |
|---|---|---|---|---|---|
| | Compound | Compound | Compound | Compound | Compound |
| | (I) | (I) | (I) | (I) | (I) |
| | 40/20 mg | 80/40 mg | 120/60 mg | 160/80 mg | 160/80 mg |
| N | 6 | 6 | 6 | 6 | 15 |
| Mean (SD) | 549.7 (146.05) | 879.7 (217.95) | 1260.8 (265.22) | 1760.0 (349.69) | 1266.5 (366.10) |
| [ng/mL] | | | | | |

Saturation of plasma PK was observed at 160 mg, the highest dose level tested. Steady-state exposure for Compound (I) was not achieved after the initial loading dose as demonstrated by the AUC_{τ} and Cₘₐₓ values, respectively. The ratios for Compound (I) Cₘₐₓ and AUC₉₆ following single oral administration of Compound (I) tablet formulation under fasted and fed conditions were approximately 0.841 and 0.942, respectively, indicating no meaningful food effect on systemic exposure. Similarly, the median tₘₐₓ was comparable under fed and fasted conditions.

In Part 1 (SRD study), Compound (I) was rapidly absorbed with a median tₘₐₓ of 0.99-3.00 h for the dose range of 5-80 mg. A secondary peak appeared for some participants at the 120 mg and 160 mg dose levels, increasing the median tₘₐₓ to 13.0 h and 13.5 h, respectively. The mean t_{1/2z} for Compound (I) ranged from 210-296 h for the 20-160 mg dose range. Cₘₐₓ and AUC₂₄ for Compound (I) increased less than proportionally. The mean Cₘₐₓ and AUC values for Compound (I) 160 mg were similar or below those for Compound (I) 120 mg, suggesting plasma PK saturation.

The mean t_{1/2z} for Compound (I) reflects the prolonged half-life of Compound (I) in humans. Species variability in the rate and extent of metabolic pathways for Compound (I) was observed in a separate study of rats, dogs, monkeys, and humans after single dose oral administration of 5-15 mg/kg Compound (I). In that study, the mean human half-life of Compound (I) (approximately 11 days) was substantially higher than the 2-4 hr mean half-life observed in rats and dogs.

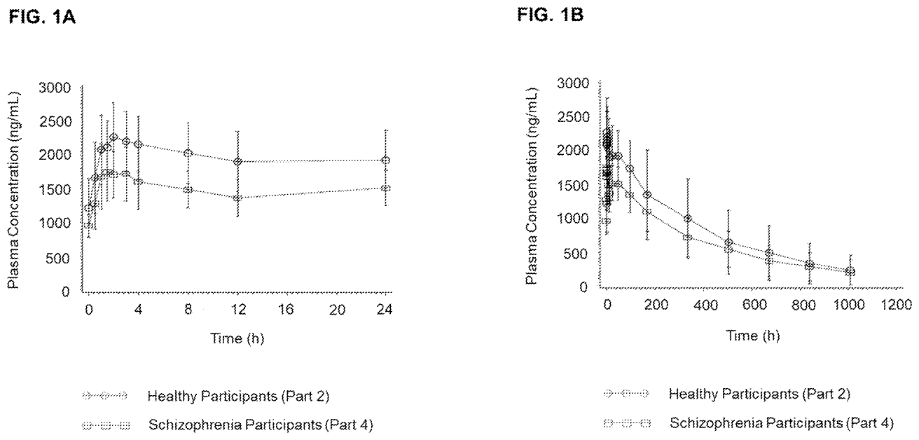

PK parameters in healthy participants following MRD (Part 2) are shown in Table 7. In Table 7, the median and min-max are presented for tₘₐₓ (noted (a) and (b) in the table, respectively). Compound (I) was also rapidly absorbed in Part 2, with a median tₘₐₓ of 1.75-3.00 h (FIG. 1). A few participants had a tₘₐₓ of approximately 48 h with Compound (I) 80 mg loading dose and 40 mg maintenance dose or Compound (I) 160 mg loading dose and 80 mg maintenance dose. Compound (I) systemic exposure at steady state (Day 22) increased approximately dose proportionally, with a mean t_{1/2z} for Compound (I) at steady state ranging from 170-302 h.

In Part 3 (relative bioavailability/food effect study), the mean Compound (I) systemic exposures for the tablet formulation were similar between fasted and fed conditions, indicating minimal food effect. The median tₘₐₓ for the Compound (I) tablet formulation (under fasted and fed states; 2.00 h) was similar to that for the Compound (I) oral suspension (1.82 h). Two participants had delayed tₘₐₓ estimates with the Compound (I) tablet (36 h and ˜24 h) compared with others within their respective treatment groups.

PK parameters for patients with stable schizophrenia (Part 4) are shown in Table 8. In Table 8, the median and minimum-maximum are presented for tₘₐₓ (noted (a) and (b) in the table, respectively), and values with a (c) noted are based on a sample size of 13 due to a missing subject who terminated early. In Part 4 (MRD study with patients with stable schizophrenia), the median tₘₐₓ for Compound (I) was 1.80 h for schizophrenia patients, similar to the median tₘₐₓ of 3.00 h for healthy participants (FIG. 1). The steady-state mean t_{1/2z} for Compound (I) was 334 h and 271 h for schizophrenia and healthy participants, respectively. Additionally, the mean t_{1/2z} was 334 h in patients with schizophrenia, compared with 210-296 h in heathy participants in Part 1 (SRD study) and 170-302 h in healthy participants in Part 2 (MRD study). Compound (I) peak systemic exposure was 23-30% lower in schizophrenia participants compared with heathy participants, and the total systemic exposure of Compound (I) was 22-27% lower for schizophrenia participants compared with heathy participants.

TABLE 7
Summary of Plasma PK Parameters in Healthy Participants Following Multiple Oral Administration of Compound (I) (Day 22)

| Cmpd | | | | | | | | | | | | | | |
|---|---|---|---|---|---|---|---|---|---|---|---|---|---|---|
| (I) | | | | | | | | | | | | | | |
| Dose | | | Cₘₐₓ | C_{av,ss} | C_{trough} | AUC₂₄ | AUC₉₆ | AUCₜ | AUC_{τ} | | | | | |
| (mg) | | tₘₐₓ | (ng/ | (ng/ | (ng/ | (h*ng/ | (h*ng/ | (h*ng/ | (h*ng/ | t_{1/2z} | | | CL/F | Vz/F |
| [N] | | (h) | mL) | mL) | ml) | mL) | mL) | mL) | mL) | (h) | R_{ac(AUC}) | R_{ac(Cmax)} | (mL/h) | (L) |
| 40/20 | Mean | 1.750 | 698.3 | 393.8 | 274.2 | 12019.8 | 42174.8 | 191160.7 | 66160.8 | 299.5 | 2.412 | 2.689 | 334.6 | 112.11 |
| [6] | SD | 1.00- | 134.51 | 148.14 | 139.00 | 2351.52 | 12605.56 | 164768.85 | 24886.74 | 304.72 | 0.8912 | 0.8256 | 106.94 | 70.222 |
| | | 2.00 | | | | | | | | | | | | |
| | % CV | — | 19.3 | 37.6 | 50.7 | 19.6 | 29.9 | 86.2 | 37.6 | 101.7 | 36.9 | 30.7 | 32.0 | 62.6 |
| 80/40 | Mean | 2.000 | 1251.7 | 889.3 | 621.3 | 24400.2 | 92680.4 | 420517.9 | 149396.0 | 301.7 | 3.028 | 2.885 | 281.9 | 123.25 |
| [6] | SD | 1.02- | 246.37 | 189.71 | 133.77 | 5323.64 | 21015.33 | 99760.95 | 31871.04 | 77.82 | 0.3136 | 0.3265 | 81.18 | 43.292 |
| | | 48.00 | | | | | | | | | | | | |
| | % CV | — | 19.7 | 21.3 | 21.5 | 21.8 | 22.7 | 23.7 | 21.3 | 25.8 | 10.4 | 11.3 | 28.8 | 35.1 |
| 120/60 | Mean | 3.000 | 1738.3 | 1206.6 | 862.5 | 35871.1 | 133990.6 | 407181.8 | 202713.0 | 169.7 | 2.782 | 2.847 | 299.1 | 73.03 |
| [6] | SD | 1.00- | 156.64 | 146.98 | 176.09 | 4306.87 | 17109.72 | 91884.01 | 24692.02 | 59.25 | 0.4423 | 0.5495 | 30.83 | 26.593 |
| | | 8.00 | | | | | | | | | | | | |
| | % CV | — | 9.0 | 12.2 | 20.4 | 12.0 | 12.8 | 22.6 | 12.2 | 34.9 | 15.9 | 19.3 | 10.3 | 36.4 |
| 160/80 | Mean | 3.000 | 2406.7 | 1751.1 | 1209.0 | 47370.4 | 181257.6 | 841279.6 | 294190.9 | 270.8 | 2.961 | 2.785 | 284.6 | 103.02 |
| [6] | SD | 2.00- | 386.56 | 410.14 | 446.31 | 10080.70 | 34794.22 | 432279.58 | 68902.90 | 130.55 | 0.6377 | 0.4384 | 66.99 | 39.140 |
| | | 48.08 | | | | | | | | | | | | |
| | % CV | — | 16.1 | 23.4 | 36.9 | 21.3 | 19.2 | 51.4 | 23.4 | 48.2 | 21.5 | 15.7 | 23.5 | 38.0 |

TABLE 8
Summary of Plasma PK Parameters in Participants with Stable Schizophrenia Following Multiple Oral Administration of Compound (I)
(Day 22)

| | | | | | | AUC₂₄ | AUC₉₆ | AUCₜ | AUC_{τ} | | | | | |
|---|---|---|---|---|---|---|---|---|---|---|---|---|---|---|
| Day | | tₘₐₓ | Cₘₐₓ | C_{av,ss} | C_{trough} | (h*ng/ | (h*ng/ | (h*ng/ | (h*ng/ | t_{1/2z} | | | CL/F | Vz/F |
| [N] | | (h) | (ng/mL) | (ng/ml) | (ng/ml) | mL) | mL) | mL) | mL) | (h) | R_{ac(AUC)} | R_{ac(Cmax)} | (mL/h) | (L) |
| 1 | Mean | 2.000 | 1266.5 | — | — | 20400.8 | 90372.3 | 148889.0 | 148889.0 | — | — | — | — | — |
| [15] | SD | 1.00- | 366.10 | — | — | 5096.46 | 22678.03 | 40389.92 | 40389.92 | — | — | — | — | — |
| | | 96.80 | | | | | | | | | | | | |
| | % CV | — | 28.9 | — | — | 25.0 | 25.1 | 27.1 | 27.1 | — | — | — | — | — |
| 22 | Mean | 1.800 | 1885.0 | 1352.6 | 950.4 | 35523.3 | 140107.5 | 659713.3 | 227230.1 | 333.8 | 3.179 | 3.028 | 365.5 | 172.39 |
| [14] | SD | 1.00- | 457.62 | 254.10 | 168.12 | 6117.26 | 24238.99 | 226076.64 | 42689.23 | 139.56 | 0.6575 | 0.6620 | 79.91 | 64.838 |
| | | 8.05 | | | | | | | | | | | | |
| | % CV | — | 24.3 | 18.8 | 17.4 | 19.1 | 17.3 | 34.3 | 18.8 | 41.8 | 20.7 | 21.9 | 21.9 | 37.6 |

### Example 2: Endogenous Dopamine Release in the Human Brain as a Pharmacodynamic Biomarker: Evaluation of Compound (I) with [¹¹C]PHNO PET

Molecular imaging techniques using dopamine-D2 receptor (D2R) radioligands have been shown to be suitable for the detection of pharmacologically-induced dopamine release in the healthy non-human primate (Dewey et al., 1993; Innis et al., 1992) and human brain (Laruelle, 2000; Laruelle et al., 1995). Depletion of synaptic dopamine with α-methyl-para-tyrosine produces PET and SPECT signal change in the opposite direction to that produced by dopamine enhancing drugs such as amphetamine or levo-DOPA (Laruelle et al., 1997; Verhoeff et al., 2002; Verhoeff et al., 2001); the magnitude of the change in signal is related to the magnitude of change in dopamine as measured by microdialysis (Breier et al., 1997; Laruelle et al., 1997). Using this methodology, patients with neuropsychiatric disorders demonstrate differences from healthy individuals (Abi-Dargham et al., 1998; Breier et al., 1997; Tedroff et al., 1996). D2R agonist radioligands such as [¹¹C]PHNO (Brown et al., 1997; Wilson et al., 2005) and [¹¹C]NPA bind primarily to the G-protein coupled (G-coupled) state of the D2R, analogous to dopamine itself, enabling high sensitivity of the agonist ligands to detect fluctuations in synaptic dopamine concentration (Narendran et al., 2004; Willeit et al., 2006).

To evaluate endogenous dopamine release in the human brain following treatment with Compound (I), human PET studies using [¹¹C]PHNO as a non-invasive method of monitoring synaptic dopamine levels were employed. In these studies, Compound (I) readily crossed the blood-brain barrier and demonstrated a dose-dependent modulation of dopamine release in healthy human volunteers. Dextroamphetamine (d-APMH) administration produced a robust reduction of [¹¹C]PHNO BP_{ND}—as would be expected from an increase in synaptic dopamine concentration. Pre-treatment with Compound (I) reduced the magnitude of post-d-APMH [¹¹C]-PHNO ΔBP_{ND}, consistent with a reduced release of extracellular dopamine post-d-AMPH administration. The plasma concentration of d-AMPH was similar in d-AMPH alone PET scan and d-AMPH+Compound (I) PET scan, suggesting the attenuation of ΔBP_{ND}^{AMPH} seen in d-AMPH+Compound (I) PET scan reflects the pharmacological effects of Compound (I). The modulation induced by 40 mg of Compound (I) was greater than that induced by 20 mg Compound (I) in all human brain regions examined, and the dose effect was significant in the putamen and the globus pallidum. The lack of significant dose effects in other brain regions were likely due to the small sample size employed in these studies.

Study Design and Subjects

Twelve healthy male volunteers (39.8±11.4 years of age) were screened and recruited into the study by Hammersmith Medicines Research, London. All imaging assessments were conducted at the Imanova Centre for Imaging Sciences (now Invicro), London, UK. The study was approved by the London—Brent Research Ethics Committee (reference 16/LO/1493), and permission to administer radioisotopes was obtained from the Administration of Radioactive Substances Advisory Committee of the UK (Ref: 630/3764/35313).

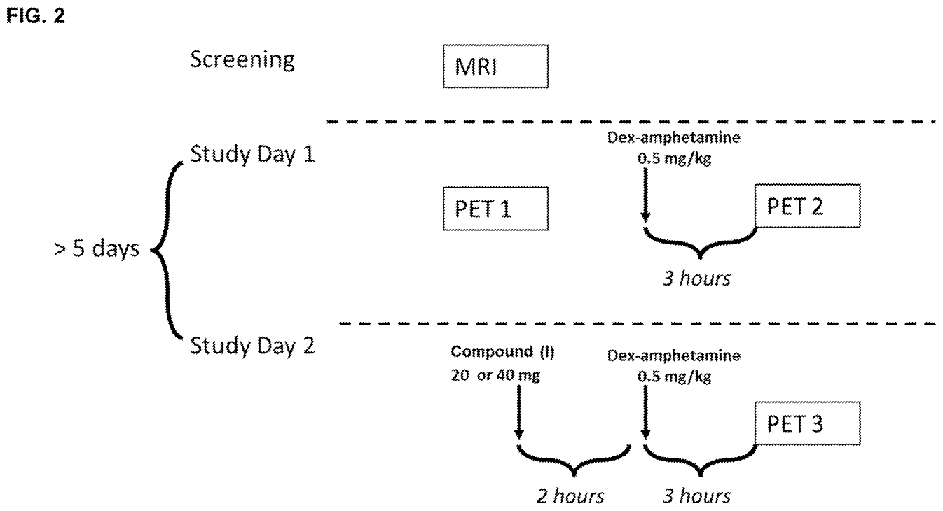

A T1-weighted magnetic resonance imaging (MRI) scan of the brain, performed at screening, was used to obtain anatomical information required for PET data analysis. Each subject underwent three [¹¹C]PHNO PET scans, one at baseline, the second approximately 3 h after a single oral dose of dex-amphetamine (d-AMPH, 0.5 mg/kg), and the third approximately 5 h after a single oral dose of Compound (I) (20 or 40 mg) and 3 h after an oral dose of dex-amphetamine (d-APMH) (0.5 mg/kg) (FIG. 2).

Two subjects received a baseline PET scan only, but not any more PET scans due to radioligand production failure. Data from these subjects were not included in analyses.

MRI Acquisition

All subjects had MRI scans (including a T1-weighted scan) acquired at screening. Scans were acquired using a Siemens 3T MRI magnet (Siemens Healthcare, Erlangen, Germany). Structural imaging data were acquired in the sagittal plane, utilizing a 3D magnetization prepared rapid gradient echo (MP-RAGE) scan with the following parameters: repetition time=2300 ms, echo delay time=2.98 ms, flip angle=9°, isotropic voxels=1.0 mm×1.0 mm×1.0 mm, 160 slices, total scanning time=5 min, 3 sec. MR scans were reviewed by a neuroradiologist to exclude any clinically relevant brain abnormalities. T1 MRI data were used as part of the PET data analysis as described below.

[¹¹C]-PHNO Preparation

[¹¹C]PHNO was made as previously described (Searle et al., 2010; Wilson et al., 2005) by the reaction of [¹¹C]-propionyl chloride with despropyl-PHNO. The final product was purified by solid phase extraction and reformulated in a solution of 10% ethanol in normal saline.

PET Data Acquisition

Subjects were positioned in the PET scanner, after the insertion of a venous cannula in an antecubital or forearm vein. Soft padding and head restraints were used to minimize head movement during data acquisition. All dynamic [¹¹C]PHNO PET scans were acquired on Siemens Biograph 6 PET/CT scanners (Hi-Rez (PET/CT1) or a TruePoint with TrueV, Siemens Healthcare, Erlangen, Germany). All scans for a particular subject were acquired on the same scanner. A low-dose computed tomography (CT) scan was performed immediately before each PET study to estimate signal attenuation. Following an intravenous bolus injection of [¹¹C]PHNO, dynamic emission data were acquired for 90 min in 26 frames of increasing duration. The dynamic images were reconstructed using Fourier rebinning and a 2D filtered discrete inverse Fourier transform algorithm with 5 mm isotropic Gaussian filter on a 128×128 matrix with 2.6 zoom giving 2 mm isotropic voxels. Appropriate corrections were applied for attenuation, randoms, and scatter.

Analysis Methods

All image data were analyzed using the MIAKAT™ (Gunn et al., 2016) software package (version 4.2.6.1), implemented using MATLAB (version R2016a; The Mathworks Inc., Natick, MA, USA), and using FSL (version 5.0.4; FMRIB, Oxford, UK; Jenkinson et al., 2005; Smith et al., 2004) functions for brain extraction and SPM12 (Wellcome Trust Centre for Neuroimaging) for image segmentation and registration.

Image Processing

Each subject's structural MRI image underwent brain extraction, grey matter segmentation and was co-registered to a standard reference space (MNI152; Grabner et al., 2006). The MNI152 template brain image and associated atlas (CIC atlas; Tziortzi et al., 2011) was nonlinearly warped to the subject's MR image to enable automated definition of regions of interest (ROIs).

The primary set of ROIs defined were the ventral striatum (VSt), putamen (Pu) and cerebellum. VSt and Pu were selected a priori based on a previous study that found the most reproducible and robust reductions in [¹¹C]-(+)-PHNO BP_{ND} following a d-AMPH challenge, in these ROIs (Shotbolt et al., 2012). The cerebellum was used as a reference region. An additional set of ROIs including the caudate nucleus (Ca), globus pallidus (GP) and substantia nigra (SN) was also evaluated.

Dynamic PET images were registered to each subject's MRI scan and corrected for motion using a frame-to-frame registration process with a normalized mutual information cost function. ROIs defined on the MRI images were applied to the dynamic PET data to derive regional time-activity curves (TACs).

Kinetic Modeling

The simplified reference tissue model (SRTM) (Lammertsma and Hume, 1996) has been demonstrated to be suitable for modeling [¹¹C]-PHNO PET data (Ginovart et al., 2006; Graff-Guerrero et al., 2010; Searle et al., 2010; Tziortzi et al., 2011; Willeit et al., 2006). Regional TAC data extracted from the PET images were fitted using a basis function implementation of the SRTM (Gunn et al., 1997) to estimate the binding potential relative to the non-displaceable component (BP_{ND}) for each PET scan, as a measure of specific binding (Innis et al., 2007).

Reduction in [¹¹C]-PHNO specific binding following a d-AMPH challenge (ΔBP_{ND}^{APMH}) was quantified as percent change in postdose BP_{ND} (BP_{ND}^{AMPH}) from baseline BP_{ND} (BP_{ND}^{BL}):

$${Δ BP}_{N D}^{A P M H} = 100 \times ( 1 - \frac{B {P}_{N D}^{A P M H}}{B {P}_{N D}^{B L}} ) . \qquad Equation 1$$

Reduction in [¹¹C]-PHNO specific binding following a combination of Compound (I) and d-AMPH (ΔBP_{ND}^{APMH+C1}) was quantified as percent change in postdose BP_{ND} (BP_{ND}^{AMPH+C1}) from baseline BP_{ND} (BP_{ND}^{BL}):

$$Δ B {P}_{N D}^{A M P H + C 1} = 100 \times ( 1 - \frac{B {P}_{N D}^{A M P H + C 1}}{B {P}_{N D}^{B L}} ) . \qquad Equation 2$$

The effect of Compound (I) (ΔΔBP_{ND}) was quantified as the percent change BP_{ND} in ΔBP_{ND}^{APMH+C1} compared to ΔBP_{ND}^{APMH}:

$$Δ Δ B {P}_{N D} = 100 \times ( 1 - \frac{Δ B {P}_{N D}^{A M P H + C 1}}{Δ B {P}_{N D}^{A M P H}} ) . \qquad Equation 3$$

All statistical comparisons were conducted using Student's t-tests for each ROI, using paired tests for within subject comparisons, and unpaired tests for between group comparisons.

Results

Image data were successfully acquired for 32 PET scans in twelve study subjects. Two subjects had only baseline [¹¹C]PHNO PET scans acquired due to radioligand synthesis failure. The remaining 10 subjects each had 3 [¹¹C]PHNO PET scans acquired. Radiochemical purity for all scans was >95%, with mean injected activity 120±26 MBq, and mean injected PHNO mass 18.4±3.0 ng/kg. The maximal difference in injected mass for the three scans for each subject was <5%.

The PET images acquired displayed the expected anatomically heterogeneous signal, consistent with both the known distribution of D2/D3 receptors and previous [¹¹C]PHNO data. Acceptable SRTM model fits to tissue TAC data was obtained for all scans and BP_{ND} values were well determined (<10% COV) for the main ROIs.

d-AMPH challenge produced reductions in BP_{ND} in all ROIs analyzed. ΔBP_{ND}^{AMPH} values (effect of d-AMPH on [¹¹C]PHNO BP_{ND}) were calculated for each ROI for PET2 (d-AMPH only scan) and ΔBP_{ND}^{AMPH+C1} (effect of d-AMPH and Compound (I)) were calculated for each ROI for PET 3 (the d-AMPH+Compound (I) scan). The effect of pre-dosing with Compound (I) on the AMPH challenge, ΔΔBP_{ND}, (the attenuation of ΔBP_{ND} by Compound (I)) was determined for each region. d-AMPH administration led to a significant reduction in BP_{ND} (ΔBP_{ND}^{AMPH}) in all ROI examined (Table 9, *=p<0.05). There was no difference in ΔBP_{ND}^{AMPH} between the 20 mg and the 40 mg Compound (I) groups. There was no relationship between the measured plasma concentration of d-AMPH and ΔBP_{ND}.

TABLE 9
ΔBP_{ND}^{AMPH} Mean (SD)

| | ΔBP_{ND}^{AMPH} (%) | | | | |
|---|---|---|---|---|---|
| | Pu | VSt | Ca | SN | GP |
| All subjects | 21.6 (5.1) * | 26.5 (7.7) * | 15.6 (5.7) * | 27.8 (10.9) * | 15.9 (7.7) * |
| 20 mg Compound (I) group | 23.6 (4.6) * | 25.4 (8.0) * | 17.8 (5.7) * | 28.4 (9.0) * | 17.6 (1.5) * |
| 40 mg Compound (I) group | 19.6 (5.3) * | 27.6 (8.2) * | 13.4 (5.3) * | 27.2 (13.6) * | 14.2 (11.1) * |

Pre-treatment with Compound (I) attenuated the effect of the d-AMPH challenge in all ROI examined. This effect was significant in the Pu and VSt, as well as the SN (Table 10, *=p<0.05). A dose effect of Compound (I) was observed in the Pu and the GP, where the ΔΔBP_{ND} in the 40 mg group was significantly greater than that in the 20 mg group (one-tailed t-tests, p<0.01). There was no relationship between the plasma concentration of Compound (I) and the ΔΔBP_{ND}.

TABLE 10
ΔΔBP_{ND} Mean (SD)

| | ΔΔBP_{ND} (%) | | | | |
|---|---|---|---|---|---|
| | Pu | VSt | Ca | SN | GP |
| All subjects | 26.4 (13.9)* | 18.2 (17.1)* | 10.3 (45.9) | 33.2 (51.8)* | 31.7 (44.6) |
| 20 mg Compound (I) | 16.4 (11.3)* | 13.8 (14.1)* | −4.2 (59.2) | 25.6 (49.7) | −3.2 (19.7) |
| 40 mg Compound (I) | 36.4 (7.7) * | 22.6 (20.3) | 24.8 (26.7) | 40.8 (58.6) | 66.6 (32.4) |

The mechanism underlying the modulation of dopamine release by Compound (I) in unclear and appears to be seen in all regions examined. GPR139 expression was shown to correlate with D2R expression in a range of brain regions, and the two receptors were shown to interact in a stable cell line (Wang et al., 2019). A similar interaction was demonstrated with the MC₃ and MC₅ receptors (Nohr et al., 2017), and u-opioid receptors (Wang et al., 2019), indicating that GPR139 can heterodimerize with other GPCRs and modulate their signaling. Thus, modulation of D2R autoreceptors by Compound (I) may reduce synaptic dopamine release.

### Example 3: A Randomized, Double-Blind, Placebo Controlled, Two-Period Cross-Over, Proof of Activity Study to Evaluate the Effects of Compound (I) on Motivational Anhedonia as Add-on to Antipsychotics in Subjects with Stable Schizophrenia

This phase 2 study assessed the mechanism of action and efficacy of Compound (I) in patients with schizophrenia experiencing moderate to severe negative symptoms. Specifically, the study was designed to evaluate the effects of Compound (I) on motivational anhedonia and cognitive function using both cognitive task performance and neuroimaging markers. To assess reward and cognition in patients with schizophrenia, a specific test battery containing both performance tests and task-induced fMRI BOLD assessments was employed. The MID was used to assess reward function in the brain, as aberrant activation on this task has been consistently reported in patients with schizophrenia, particularly in regions modulated by the habenula such as the ventral tegmental area (VTA) (Nielsen et al., 2012) and nucleus accumbens (NAcc) (Radua et al., 2015). Importantly, differences in NAcc and ventral striatal brain activity during the MID task have also been directly linked with the severity of negative symptoms (Juckel et al., 2006, Radua et al., 2015). Outside the scanner, cognitive function was assessed using the Brief Assessment of Cognition in Schizophrenia (BACS) tool (Keefe et al., 2004).

Compound (I) was safe and well-tolerated in subjects with stable schizophrenia in the phase 2 study. Reward task activation was modulated by Compound (I) not acutely, but after 14 days of exposure. Changes in striatal response have been linked to changes in both positive (Nielsen et al., 2012) and negative (Juckel et al., 2006, Radua et al., 2015) symptoms. Divergent effects of Compound (I) on cerebral blood flow were also observed, with widespread and robust reductions at Day 1 followed by increases at Day 14 compared to placebo, mirroring the opposing directions of the effect of the API on the reward system response. BACS performance was not seen to be affected by Compound (I) treatment, and performance on this task was not correlated with any of the changes seen in the extracted BOLD values.

An exploratory analysis of the changes in symptomatology as measured by the PANSS and BNSS did not reveal any relationship between reward anticipation response and severity of symptoms in this study, apart from the negative association between anhedonia and ventral striatal response at Day 14 that did not survive multiple comparisons correction. However, studies that have reported associations between symptom change and ventral striatal responses typically assess patients over a longer time scale (such as, e.g., 6 weeks). Thus, a longer dosing period may be required to understand the impact of these imaging changes on symptoms and behavior.

Methodology

This study was a randomized, double-blind, placebo-controlled, 2-period, crossover phase 2 study to evaluate the PD effects, safety, tolerability, and pharmacokinetic (PK) of single doses of oral Compound (I) in adult subjects with schizophrenia, particularly negative symptoms characterized by reduced motivation. The primary objectives were: (1) to determine whether motivation/reward deficits observed in schizophrenia are attenuated by add-on Compound (I) administration to antipsychotics in subjects with stable schizophrenia; and (2) to determine whether cognitive impairment associated with schizophrenia is improved by add-on Compound (I) administration to antipsychotics in subjects with stable schizophrenia. The secondary objective was to determine the safety and tolerability of Compound (I) as an add-on therapy to antipsychotics in subjects with stable schizophrenia.

The study consisted of 2 treatment periods, with a single dose of study drug administered in each period. There was a 35 day (+7 days) washout interval between the 2 doses to reduce the potential for residual Compound (I) to impact the PD endpoints. Treatment Period 2 began at the end of the Treatment Period 1 washout. Because subjects were not confined in this study, they came to the clinic for the following visits: screening visit (between Days −35 to −2) covering full medical and psychiatric examinations; baseline assessments visit (Day −1 in each treatment period), covering motivation/reward and cognitive testing baseline, except functional magnetic resonance imaging (fMRI); dosing and first testing visit (Day 1 in each treatment period); second testing visit (Day 14 in each treatment period); follow-up visit (Day 49 [±4 days] post-dose for treatment period 2 only); end of period 1 visit (Day 35 [+7 days] post-dose in period 1); study exit occurred at the final visit 77±7 days post-period 2 dose.

The study population included subjects with stable schizophrenia aged 18 to 60 years, inclusive who met the study inclusion and exclusion criteria. On Day 1 of Period 1, eligible subjects were randomized in a ratio of 1:1 to 1 of the 2 treatment sequences and received each study drug according to the randomized sequence group. The randomization was stratified by the sites. The initial dose of Compound (I) was 40 mg and was adjusted up to 160 mg based on available PK and safety data from emerging first-in-human cohorts. The decision criteria were predefined in the statistical analysis plan (SAP) before unblinding.

During the screening visit (Days −35 to −2), subjects meeting Diagnostic and Statistical Manual of Mental Disorders, 5th Edition (DSM-5) criteria for schizophrenia and on stable antipsychotic therapy for at least 2 months completed medical and psychiatric examinations, electrocardiogram (ECG), and laboratory safety tests. At the baseline assessments visit (Day −1) for Period 1, subjects completed practice sessions of the motivation/reward and cognitive battery in order to minimize potential practice effects at subsequent treatment visits and be familiarized to the magnetic resonance imaging (MRI) tasks. At both study periods, subjects were also tested for Day −1 baseline assessments (except fMRI). Practice sessions were carried out before the baseline assessments in Period 1 only. Subjects meeting all inclusion criteria and no exclusion criteria were administered study medication or placebo in the clinic based on the randomization schedule on Day 1 of Treatment Periods 1 and 2.

On Day 1 of each treatment period, following study drug administration, and commencing at approximately 2 h post-dose, subjects underwent PD tests for a total of approximately 4 h. Blood samples were collected at defined times after dosing to assess the PK of Compound (I). After the last PK collection at 6 h post-dose, subjects left the clinic. On Day 14 of each Treatment Period, subjects took the second set of PD tests along with a single time point PK collection. Samples for PK were also collected on Day 49 and at the final visit of the second treatment period. PD testing were performed in 3 blocks, separated by 2 breaks. The first block comprised the Grip Effort Task, the Progressive Ratio Test, and the Brief Assessment of Cognition in Schizophrenia (BACS). The BACS is a reliable and sensitive test battery specifically designed to efficiently assesses important cognitive deficits in patients with schizophrenia; accordingly, the BACS is suited for repeated testing in a clinical trial setting (Keefe et al., 2004). It has shown superior internal consistency and test-retest reliability to alternative tests of neuropsychological status (Chianetta et al., 2008). The following domains of cognitive function found to be consistently impaired in schizophrenia were assessed in this study: verbal memory; working memory; motor function; attention and processing speed; verbal fluency; and executive function. A composite score was calculated from each of the subset scores and used as a single endpoint for the assessment.

After a break, subjects took the second block that comprised a battery of imaging tests; namely, brain perfusion using non-contrast arterial spin labeling MRI and fMRI scans which monitor changes in blood oxygen level-dependent (BOLD) signal. The fMRI was run in resting state without a task (resting state fMRI) and with 2 tasks (2-task fMRI) using the monetary incentive delay (MID) Reward Task and a Set-Shifting Task. After a break, subjects took the third block which comprised the Effort Expenditure for Rewards Task and the Empathic Accuracy Task. Subjects also received the Positive and Negative Syndrome Scale (PANSS), the Brief Negative Syndrome Scale (BNSS), and the Clinical Global Impression (CGI) Scales (CGI-Severity and CGI-Improvement) as a part of the PD assessment.

Optional pharmacogenomics assessments were performed from the blood samples collected on Day 1 (before Compound (I) or placebo administration) for DNA in Period 1 only, and in both treatment periods for RNA on Day 1 (before Compound (I) or placebo administration) and on Day 14.

Participants

23 subjects were enrolled in the double-blind treatment period, with 23 subjects in the safety analysis set, 22 subjects in the PK analysis set, and 23 subjects in the PD analysis set. One subject voluntarily discontinued after receiving Compound (I) because of inability to complete the MRI procedure, and one subject discontinued after receiving placebo due to failure to meet inclusion criteria with a positive urine drug screen.

66 adult male or female (of nonchildbearing potential, not lactating or nursing) with schizophrenia aged between 18 to 60 were screened for the study, recruited from mental health community services in the Greater London area. Screening procedures were conducted between 2 and 35 days prior to the first dosing and scanning session. Inclusion criteria required a diagnosis of schizophrenia as defined in the DSM-5 by the Mini International Neuropsychiatric Interview (MINI), without a current and active diagnosis of a significant psychiatric illness (e.g., bipolar disorder, depression, GAD, etc.) other than schizophrenia, per DSM-5 (including meeting criteria for substance use disorder or history of alcohol abuse within 1 month prior to screening). Participants must have been treated with a stable dose of an antipsychotic other than clozapine for at least 2 months prior to screening, and score ≤90 on the PANSS. To ensure presence of negative symptomology, a negative symptoms factor score (NSFS) of ≥15 was required on the PANSS at screening (N1 Blunted Affect, N2 Emotional Withdrawal, N3 Poor Rapport, N4 Passive Social Withdrawal, N6 Lack of Spontaneity and Flow of Conversation, G7 Motor Retardation, and G16 Active Social Avoidance). To ensure stability of symptoms, the PANSS was re-administered on the day prior to randomization and participants with NSFS or total PANSS scores that changed more than 20% between screening and Day −1 were excluded.

Participants who were deemed at risk of suicide during the screening window according to the Columbia Suicide Severity Rating Scale (CSSRS) (positive answers on question 4 or 5 ideation or any suicidal behavior for the last 6 months or a suicide attempt within 6 months before screening) on were also excluded. Subjects were also excluded if they had a history or reasonable suspicion of disease that would contraindicate taking Compound (I) or history that might interfere with the conduct of the study or had a current diagnosis of a significant psychiatric illness other than schizophrenia and in an acute phase/episode.

Assessment of general physical suitability for the study included medical history, a full physical exam, vital signs, electrocardiogram, blood and urine chemistry profiles, serology (HIV1, HIV2, hepatitis B and/or hepatitis C and pregnancy), tests for alcohol and drugs of abuse, and concomitant medication. Only participants judged to be in good physical health by the study medic were included. Exclusion criteria included history of cancer, values >ULN on liver function tests, or a positive drug of abuse or alcohol breath test on the screening or study days. Participants also had to agree to adhere to contraception guidelines while taking part to avoid pregnancy during the study.

An MRI scanning visit within the screening window was conducted to familiarize the participant with the scanning session. T1 and T2 weighted images were acquired and inspected by a neuroradiologist to rule out any structural brain abnormalities or other abnormalities that would interfere with interpretation of functional brain imaging results.

23 (17 male) screened participants met eligibility criteria and were randomized into the double-blind treatment study (age range 21-60, mean age: 43.8±12.1 years). Most subjects were Black or African American, British African, Black British or Black British Caribbean (69.6%). The mean body mass index was 30.3 kg/m². Medical history was unremarkable across subjects, and the most frequent concurrent medical conditions were hypertension, back pain, muscle spasms, seasonal allergy, and asthma.

Duration of Treatment

The study consisted of 2 treatment periods, in a randomized, double-blind, placebo-controlled, crossover design, in which participants received a single dose of Compound (I) or placebo during each period. A 35-42 day washout interval was given between the 2 doses given the long half-life of Compound (I).

Participants that passed screening procedures and were suitable to take part attended the NIHR Clinical Research Facility at King's College Hospital, London for the following visits:

Baseline Assessments Visit (Day −1 in each Treatment Period): Final screening assessments were completed (blood tests, urinalysis, drug, and alcohol screen) as well as cognitive baseline practice and testing, baseline psychiatric scales and familiarization with scanner tasks.

Dosing and First Testing Visit (Day 1 in each Treatment Period): Following a physical health check by a study physician (drug and alcohol screen, ECG, vitals, physical exam) a single dose of either Compound (I) or placebo was administered between 10:30 AM and 12:00 PM. The BACS was administered approximately 2.5 h post dose, and the fMRI session began 3.5 h post dose.

Second Testing Visit (Day 14 in each Treatment Period): Two weeks after dosing day, participants returned to complete the BACS and another fMRI session, identical to the initial session. These were conducted at the same time of day as the Day 1 testing sessions.

End of Period 1 Visit (Day 35-42) and Start of Period 2: An single end of Period 1/start of Period 2 visit acted as the baseline assessment visit (Day −1) for Period 2, after which Day 1 and Day 14 visits were repeated as above with the participants receiving the opposite treatment to that received in period 1.

Follow-up Visit (Day 49 [±4 days] post Period 2 dose): A safety follow up involving physical and psychiatric health assessment.

Study Exit Visit (77±7 days post Period 2 dose): As Day 49 follow up.

Dosing

Dosing involved participants drinking a single bottle of oral suspension, containing either Compound (I) (40 or 160 mg Compound (I) mixed with 70 mL Tween/MC vehicle) or placebo (70 mL of Tween/MC vehicle) (Table 11). Participants were instructed to drink the full contents of the bottle, which was then rinsed twice with 35 mL water, which participants also drank. The initial dose level selected was 40 mg, which was adjusted to 160 mg following a planned interim analysis of available PK and safety data from emerging first-in-human cohort data. This resulted in the first seven participants receiving a 40 mg dose of Compound (I), and the remaining 17 participants receiving a 160 mg dose.

TABLE 11
Study Drugs, Dose, and Mode of Administration

| | Product Dose | | Mode of |
|---|---|---|---|
| Study Drug(s) | Strength and Form | Study Dosage | Administration |
| Compound (I) | 40 mg oral | 40 mg single | Oral suspension |
| | suspension | dose | |
| Compound (I) | 40 mg oral | 160 mg single | Oral suspension |
| | suspension | dose | |
| Placebo | 0 mg oral | 0 mg | Oral suspension |
| | suspension | | |

PD Analysis

The PD analysis set consisted of all subjects who had at least 1 dose of study drug and had at least 1 evaluable primary or exploratory measurement.

The BOLD signal in the average of the left and right ventral striatum activation in the MID Reward Task 3.5 h post-dose on Day 1 and the BACS composite score on Day 14 were the primary PD measures.

The BACS consisted of items across 6 subtests: Verbal Memory, Digit Sequencing, Token Motor, Verbal Fluency, Symbol Coding, and Tower of London.

The observed BOLD signal was summarized (N, mean, median, SD, minimum, and maximum) by treatment (Compound (I) (overall and by dose) and placebo) and time. The BACS composite score was summarized for baseline, post-dose, and change from baseline by treatment, dose, and time.

Each of the primary PD measures was analyzed using a Bayesian normal linear model with effects for sequence, period, treatment, time (as a categorical variable), the treatment-by-time interaction, subject within treatment sequence, and baseline (for BACS only). For the BOLD fMRI, the observed value was the response variable in the model. For the BACS, the observed and the change from baseline were modeled separately. Missing values were not imputed and were not included in the model under a missing at random assumption. A diffuse normal distribution with mean zero and variance 106 was used as a prior for the regression coefficients and a diffuse inverse gamma with shape and scale parameters of 0.01 for the residual variance.

The posterior mean, SD, and the 90% credible interval (highest posterior density interval) were extracted for each treatment and time, along with the posterior mean treatment.

The Bayesian analysis and the linear mixed effects model for repeated measures analysis were performed for BOLD fMRI MID and BACS. The linear mixed effects model for repeated measures analysis were performed for the BNSS and the Grip Effort Task. Summary tables and data listings of the observed values and the change from baseline (except for BOLD fMRI MID) for each PD measure were provided.

The criteria for a positive result at the IA was 70% posterior probability (or greater) of a difference between Compound (I) and placebo >0.09 in ventral striatum activation in the MID fMRI at 3.5 h post-dose on Day 1 or 70% (or greater) posterior probability of a difference between Compound (I) and placebo >2 points in the BACS composite score at Day 14.

PK Results

The PK set consisted of all subjects who received at least 1 dose of study drug and had at least 1 measurable plasma concentration.

Compound (I) plasma concentrations were tabulated and summarized by descriptive statistics at each scheduled time point (mean, median, SD, percent coefficient of variation, minimum, and maximum) for each treatment and dose level. Individual subject plasma concentration data were listed.

The plasma PK parameters of Compound (I) were determined from the concentration-time profiles for all evaluable subjects. Actual sampling times, rather than scheduled sampling times, were used in all computations involving sampling times. The following PK parameters were calculated using non-compartmental analysis using Phoenix WinNonLin (version 6.3 or higher): area under the plasma concentration-time curve from time 0 to time t, area under the plasma concentration-time curve from time 0 to infinity (AUC_{∞}), maximum observed plasma concentration (Cₘₐₓ), time of first occurrence of Cₘₐₓ (tₘₐₓ), and terminal disposition phase half-life.

Following oral administration of a single dose of Compound (I) (40 or 160 mg) to schizophrenia subjects, Compound (I) was rapidly absorbed with a median tₘₐₓ of 1.83 and 1.92 h for the 40 and 160 mg dose levels, respectively. A dose-dependent increase in Compound (I) plasma exposure (Cₘₐₓ and area under the plasma concentration-time curve) was observed. Mean Cₘₐₓ values increased 2.8-fold (549 and 1523 ng/mL for 40 and 160 mg, respectively), and mean AUC_{∞} values increased 3.5-fold (217 and 763 h·μg/mL for 40 and 160 mg, respectively) for a 4-fold increase in Compound (I) dose.

Safety

The safety analysis set consisted of all subjects who were enrolled and received at least 1 dose of study drug. Safety measurements included adverse events, physical examinations, vital signs, clinical laboratory evaluations, ECGs, and C-SSRS.

Subjects experienced TEAEs at similar frequencies in the placebo and Compound (I) treatment groups, and there was no pattern of differences in any individual TEAE between Compound (I) and placebo. No subjects discontinued study medication due to TEAEs, and there were no deaths or serious adverse events. There were no TEAEs graded severe. There were no concerning trends in laboratory tests, vital signs, ECGs, physical examinations or C-SSRS, with Compound (I) treatment relative to placebo.

Overall, Compound (I) was safe and well-tolerated in subjects with stable schizophrenia

Primary Objective Results

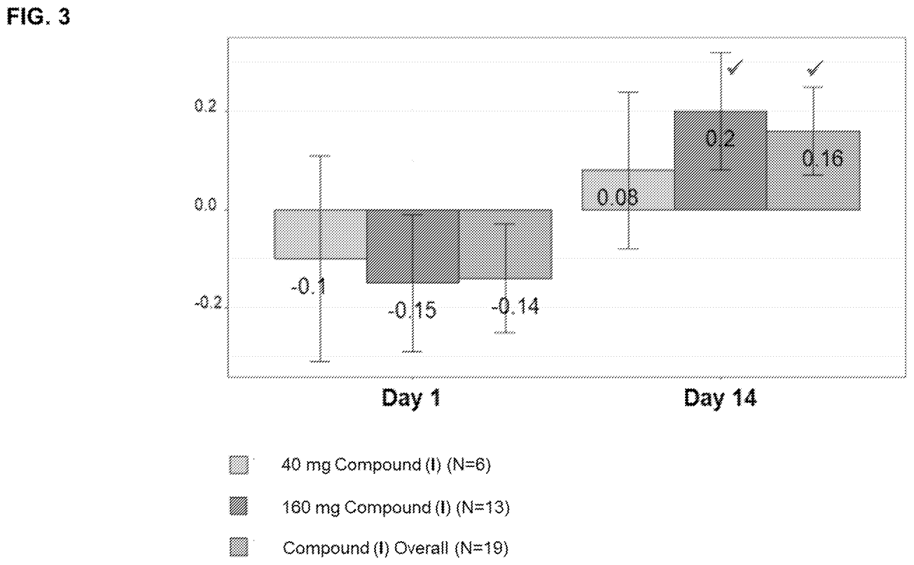

On Day 1, there was a decrease in ventral striatal activation with Compound (I) relative to placebo. The posterior mean and SD for the difference fMRI BOLD endpoint in placebo and in Compound (I) dose levels 40 mg, 160 mg and overall on Day 1 were −0.1 (0.21), −0.15 (0.14) and −0.14 (0.11) correspondingly (FIG. 3). The posterior probabilities that the increase over placebo in the fMRI BOLD endpoint at Day 1 is greater than 0.09, were 0.1706, 0.0373 and 0.0209. According to the study design, the results did not demonstrate the superiority of Compound (I) over placebo.

On Day 14, there was a dose-related increase in activation relative to placebo (FIG. 3). The fMRI BOLD endpoint, average activation of the left and right ventral striatum region of interest (ROI) during the MID Reward Task on Day 14 met pre-defined ‘positive’ signal criteria for the 160 mg dose and overall. The posterior probability that the treatment difference on BOLD MID fMRI for these dose levels are greater than 0.09 were 0.4657, 0.8314 and 0.7913, which demonstrate superiority of Compound (I) over placebo on Day 14. However, this criteria set was proposed for the results from Day 1, and thus, overall, this study did not meet “Positive” criteria.

The posterior mean (SD) for the difference between the BACS composite score in placebo and Compound (I) dose levels 40 mg, 160 mg, and overall on Day 14 were −0.23 (2.95), −0.84 (1.87), and −0.61 (1.50) correspondingly. The posterior probability that the population increase over placebo in the BACS composite score at Day 14 is greater than 2 points, were 0.2079, 0.0633 and 0.0413. According to the study design, the result was not sufficient to demonstrate the superiority of Compound (I) over placebo.

Average Global Cerebral Blood Flow

The literature of blood flow changes in schizophrenia populations is mixed, but a general pattern appears to be reductions in cortical grey matter blood flow when compared to healthy controls, with both increases and decreases in subcortical regions also reported (Zhu et al., 2017).

Whole brain maps of regional cerebral blood flow (CBF) were obtained using an ASL methodology previously reported (Hawkins et al., 2018; Dai et al., 2008). Briefly, images were acquired using a pseudo-continuous Arterial Spin Labelling sequence (pCASL) with a multi-shot, segmented 3D stack of axial spirals (8-arms) readout with a resultant spatial resolution of 2×2×3 mm. Three control-label pairs were used to derive a perfusion weighted difference image. The labelling RF pulse had duration of 1.5 s and a post-labelling delay of 1.5 s. The sequence included background suppression for optimum reduction of the static tissue signal. A Proton Density image was acquired in 48 s using the same acquisition parameters to compute the CBF map in standard physiological units (mL blood/100 g tissue/min). Two runs were acquired per visit and averaged following preprocessing.

The T2-weighted image was co-registered to the T1-weighted image prior to creation of the DARTEL template, and each session's Proton Density image was then co-registered to the T2-weighted image. The parameters for this transformation were then applied to the CBF maps, and the DARTEL flow field applied to normalize to CBF maps to MNI space, before smoothing with a 6 mm FWHM kernel.

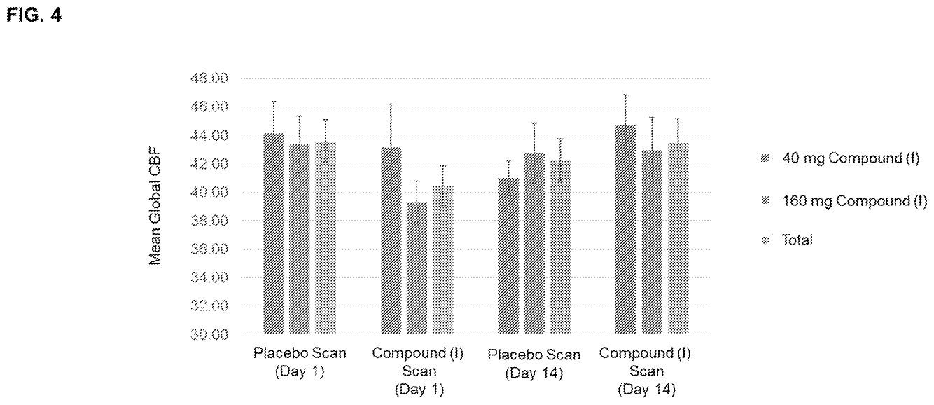

Average global cerebral blood flow (CBF) was extracting using an SPM MNI whole brain mask in MarsBar, and was analyzed at each time point with a mixed two-way ANOVA (treatment (placebo/drug) within subject factor, dose level (40 mg/160 mg) between-subject factor) (FIG. 4). At Day 1, there was a significant effect of treatment (F(1, 18)=5.978, p=0.025) but no treatment*dose interaction (F(1, 18)=2.306, p=0.146). At Day 14, there was no significant effect of treatment (F(1, 15)=3.558, p=0.079) and no treatment*dose interaction (F(1, 15)=2.973, p=0.105).

### Example 4: Compound (I) Tablet Formulations

Tablets comprising 40 mg of at least one compound chosen from Compound (I) and pharmaceutically acceptable salts thereof were developed for use in the relative bioavailability/food effect study discussed in Example 1. These tablets possessed the composition shown in Table 12, where (a) denotes components removed during processing and q.s. refers to quantum sufficit. Components with a (b) notation are components of the premixed coating materials OPADRY Red 03F45081 and OPADRY Yellow 03F42240.

A wet granulation process has been employed to prepare tablets comprising at least one compound chosen from Compound (I) and pharmaceutically acceptable salts thereof. The process consists of a conventional manufacturing method that is widely used in the pharmaceutical industry. The process conditions have been set based on historical knowledge of the manufacturing process for similar formulations using the same unit operations.

TABLE 12
Composition of 40 mg Tablets in Example 1

| | | Reference to | |
|---|---|---|---|
| | Components | Standard | Amount (mg) |
| Core | Compound (I) | — | 40 |
| tablets | Mannitol | USP | 203 |
| | Microcrystalline Cellulose | NF | 30 |
| | Hydroxypropyl Cellulose | NF | 9 |
| | Croscarmellose Sodium | NF | 15 |
| | Magnesium Stearate | NF | 3 |
| | Purified Water ^{(a)} | USP | q.s. |
| Film | Hypromellose 2910 ^{(b)} | USP | 9 |
| coating | Polyethylene Glycol 8000 ^{(b)} | NF | 2 |
| | Titanium Dioxide ^{(b)} | USP | 1 |
| | Ferric Oxide, Red ^{(b)} | NF | 0.1 |
| | Ferric Oxide, Yellow ^{(b)} | NF | 0.1 |
| | Purified Water ^{(a)} | USP | q.s. |

| | |
|---|---|
| Total | 312.2 |

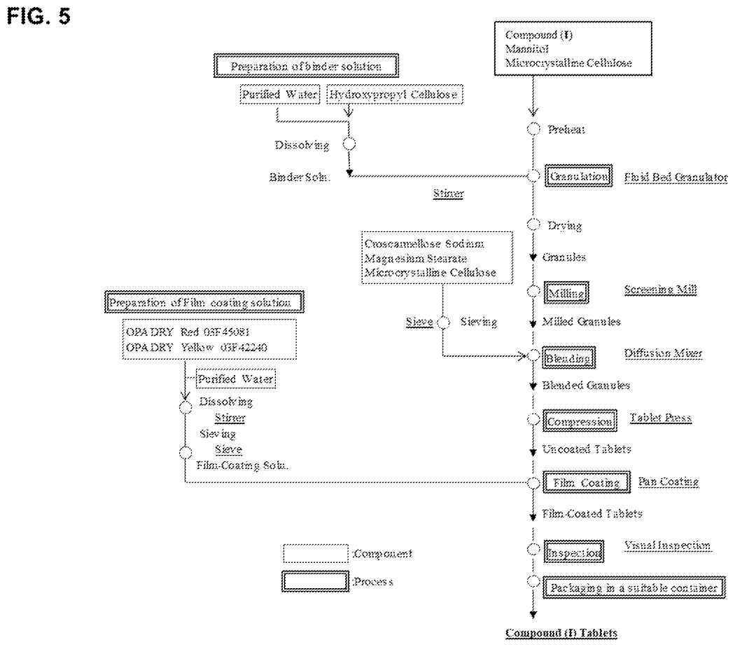

To prepare tablets comprising at least one compound chosen from Compound (I) and pharmaceutically acceptable salts thereof, a binder solution was prepared by dissolving hydroxypropyl cellulose in purified water by stirring. At least one compound chosen from Compound (I) and pharmaceutically acceptable salts thereof, mannitol, and microcrystalline cellulose were charged in a fluid bed granulator. The charged powders were granulated by spraying the binder solution in the fluid bed granulator. The granules were dried in the fluid bed granulator. The dried granules were then milled through a screening mill or sieved through a suitable screen. Next, the milled granules were blended with croscarmellose sodium, magnesium stearate, and microcrystalline cellulose in a diffusion mixer, and then the blended granules were compressed into tablets using a tablet press. The tablets were coated with an aqueous film coating solution containing OPADRY Red 03F45081 and OPADRY Yellow 03F42240 by a pan coating and inspected visually or by an automated inspection machine. A manufacturing flow diagram is shown in FIG. 5.

Additional, non-limiting examples of tablet compositions comprising at least one compound chosen from Compound (I) and pharmaceutically acceptable salts thereof prepared according to the above protocol include those listed in Table 13. The example compositions of Table 13 contain 20 mg or 40 mg of at least one compound chosen from Compound (I) and pharmaceutically acceptable salts thereof.

TABLE 13
Additional Example Tablet Compositions

| | | Reference to | Amount (mg) | |
|---|---|---|---|---|
| | Components | Standard | 20 mg | 40 mg |
| Core | Compound (I) | — | 20 | 40 |
| tablets | Mannitol | USP | 142 | 122 |
| | Microcrystalline Cellulose | NF | 20 | 20 |
| | Hydroxypropyl Cellulose | NF | 6 | 6 |
| | Croscarmellose Sodium | NF | 10 | 10 |
| | Magnesium Stearate | NF | 2 | 2 |
| | Purified Water ^{(a)} | USP | q.s. | q.s. |
| Film | Hypromellose 2910 ^{(b)} | USP | 6 | 6 |
| coating | Polyethylene Glycol 8000 ^{(b)} | NF | 1.3 | 1.3 |
| | Titanium Dioxide ^{(b)} | USP | 0.7 | 0.7 |
| | Ferric Oxide, Red ^{(b)} | NF | 0.067 | 0.067 |
| | Ferric Oxide, Yellow ^{(b)} | NF | 0.067 | 0.067 |
| | Purified Water ^{(a)} | USP | q.s. | q.s. |

| | | |
|---|---|---|
| Total | 208.1 | 208.1 |

What is claimed is:

1. A method of treating schizophrenia comprising administering to a patient in need thereof more than 80 mg of at least one compound chosen from Compound (I): 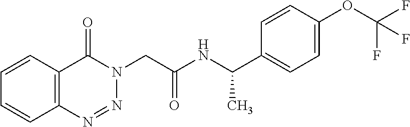 [SMILES: C[C@H](NC(=O)Cn1nnc2ccccc2c1=O)c1ccc(OC(F)(F)F)cc1]
and pharmaceutically acceptable salts thereof.

2. The method of claim 1, wherein more than 100 mg of at least one compound chosen from Compound (I) and pharmaceutically acceptable salts thereof is administered.

3. The method of claim 1 or 2, wherein 120 mg of at least one compound chosen from Compound (I) and pharmaceutically acceptable salts thereof is administered.

4. The method of claim 1 or 2, wherein 160 mg of at least one compound chosen from Compound (I) and pharmaceutically acceptable salts thereof is administered.

5. The method of any one of claims 1 to 4, wherein the at least one compound chosen from Compound (I) and pharmaceutically acceptable salts thereof is effective to achieve a mean Cₘₐₓ in the patient of at least 500 ng/mL.

6. The method of any one of claims 1 to 5, wherein the at least one compound chosen from Compound (I) and pharmaceutically acceptable salts thereof is effective to achieve a mean Cₘₐₓ in the patient of not more than 2500 ng/mL.

7. The method of any one of claims 1 to 6, wherein the at least one compound chosen from Compound (I) and pharmaceutically acceptable salts thereof is effective to achieve a mean Cₘₐₓ in the patient from 500 ng/mL to 2500 ng/mL.

8. The method of claim 4, wherein the at least one compound chosen from Compound (I) and pharmaceutically acceptable salts thereof is effective to achieve a mean Cₘₐₓ in the patient from 50% to 150% of 1267 ng/mL.

9. The method of any one of claims 1 to 8, wherein the at least one compound chosen from Compound (I) and pharmaceutically acceptable salts thereof is orally administered.

10. The method of any one of claims 1 to 9, wherein the at least one compound chosen from Compound (I) and pharmaceutically acceptable salts thereof is administered in at least one tablet.

11. The method of any one of claims 1 to 9, wherein the at least one compound chosen from Compound (I) and pharmaceutically acceptable salts thereof is administered in an oral suspension.

12. The method of any one of claims 1 to 11, wherein the at least one compound chosen from Compound (I) and pharmaceutically acceptable salts thereof is administered without food.

13. The method of any one of claims 1 to 11, wherein the at least one compound chosen from Compound (I) and pharmaceutically acceptable salts thereof is administered with food.

14. The method of any one of claims 1 to 13, further comprising administering at least one additional active pharmaceutical ingredient.

15. The method of claim 14, wherein the at least one additional active pharmaceutical ingredient is chosen from antipsychotics.

16. The method of any one of claims 1 to 15, comprising treating at least one negative symptom of schizophrenia.

17. The method of claim 16, wherein the at least one negative symptom of schizophrenia is chosen from anhedonia, loss of motivation, and reduced interest in social interaction.

18. The method of any one of claims 1 to 17, comprising treating at least one cognitive symptom of schizophrenia.

19. The method of claim 18, wherein the at least one cognitive symptom of schizophrenia is chosen from impaired verbal memory, impaired working memory, impaired motor function, impaired attention and processing speed, impaired verbal fluency, and impaired executive function.

20. The method of any one of claims 1 to 19, comprising modulating reward anticipation related brain activity in the patient.

21. The method of any one of claims 1 to 20, comprising modulating cerebral blood flow in the patient.

22. The method of any one of claims 1 to 21, comprising increasing ventral striatal activity during reward anticipation in the patient.

23. The method of any one of claims 1 to 22, comprising modulating dopamine release in the patient.

24. A method of treating schizophrenia comprising administering to a patient in need thereof:
a loading dose comprising more than 20 mg of at least one compound chosen from Compound (I): 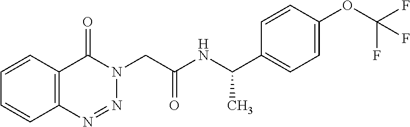 [SMILES: C[C@H](NC(=O)Cn1nnc2ccccc2c1=O)c1ccc(OC(F)(F)F)cc1]
and pharmaceutically acceptable salts thereof; and
at least one weekly maintenance dose comprising at least one compound chosen from Compound (I) and pharmaceutically acceptable salts thereof.

25. The method of claim 24, wherein the at least one weekly maintenance dose is administered 5 to 9 days after the loading dose.

26. The method of claim 24 or 25, wherein the at least one weekly maintenance dose is administered 7 days after the loading dose.

27. The method of any one of claims 24 to 26, wherein the loading dose comprises 40 mg of at least one compound chosen from Compound (I) and pharmaceutically acceptable salts thereof.

28. The method of any one of claims 24 to 26, wherein the loading dose comprises 80 mg of at least one compound chosen from Compound (I) and pharmaceutically acceptable salts thereof.

29. The method of any one of claims 24 to 26, wherein the loading dose comprises 120 mg of at least one compound chosen from Compound (I) and pharmaceutically acceptable salts thereof.

30. The method of any one of claims 24 to 26, wherein the loading dose comprises 160 mg of at least one compound chosen from Compound (I) and pharmaceutically acceptable salts thereof.

31. The method of any one of claims 24 to 30, wherein the loading dose is effective to achieve a mean Cₘₐₓ in the patient of at least 250 ng/mL.

32. The method of any one of claims 24 to 31, wherein the loading dose is effective to achieve a mean Cₘₐₓ in the patient of not more than 2500 ng/mL.

33. The method of any one of claims 24 to 32, wherein the loading dose is effective to achieve a mean Cₘₐₓ in the patient from 500 ng/mL to 2500 ng/mL.

34. The method of claim 30, wherein the loading dose is effective to achieve a mean Cₘₐₓ in the patient from 50% to 150% of 1267 ng/mL.

35. The method of any one of claims 24 to 34, wherein the at least one weekly maintenance dose is half the loading dose.

36. The method of claim 30 or 34, wherein the at least one weekly maintenance dose comprises 80 mg of at least one compound chosen from Compound (I) and pharmaceutically acceptable salts thereof.

37. The method of any one of claims 24 to 36, wherein the at least one weekly maintenance dose is effective to achieve a mean Cₘₐₓ at steady state in the patient of at least 400 ng/mL.

38. The method of any one of claims 24 to 37, wherein the at least one weekly maintenance dose is effective to achieve a mean Cₘₐₓ at steady state in the patient of not more than 3000 ng/mL.

39. The method of any one of claims 24 to 38, wherein the at least one weekly maintenance dose is effective to achieve a mean Cₘₐₓ at steady state in the patient from 400 ng/mL to 3000 ng/mL.

40. The method of claim 36, wherein the at least one weekly maintenance dose is effective to achieve a mean Cₘₐₓ at steady state in the patient from 50% to 150% of 1885 ng/mL.

41. The method of any one of claims 24 to 40, wherein the at least one weekly maintenance dose is effective to achieve a mean C_{av,ss} in the patient of at least 200 ng/mL.

42. The method of any one of claims 24 to 41, wherein the at least one weekly maintenance dose is effective to achieve a mean C_{av,ss} in the patient of not more than 2500 ng/mL.

43. The method of any one of claims 24 to 42, wherein the at least one weekly maintenance dose is effective to achieve a mean C_{av,ss} in the patient from 200 ng/mL to 2500 ng/mL.

44. The method of claim 36 or 40, wherein the at least one weekly maintenance dose is effective to achieve a mean C_{av,ss} in the patient from 50% to 150% of 1353 ng/mL.

45. The method of any one of claims 24 to 44, wherein the mean AUC_{τ} at steady state in the patient is not more than 400,000 ng·h/mL.

46. The method of any one of claims 24 to 45, the mean AUC_{τ} at steady state in the patient is not more than 300,000 ng·h/mL.

47. The method of any one of claims 24 to 46, wherein the mean AUC_{τ} at steady state in the patient is from 30,000 ng·h/mL to 400,000 ng·h/mL.

48. The method of any one of claims 36, 40, or 44, wherein the mean AUC_{τ} at steady state in the patient is from 50% to 150% of 227,230 ng·h/mL.

49. The method of any one of claims 24 to 48, wherein the loading dose is orally administered.

50. The method of any one of claims 24 to 49, wherein the loading dose is administered in at least one tablet.

51. The method of any one of claims 24 to 49, wherein the loading dose is administered in an oral suspension.

52. The method of any one of claims 24 to 51, wherein the loading dose is administered without food.

53. The method of any one of claims 24 to 51, wherein the loading dose is administered with food.

54. The method of any one of claims 24 to 53, wherein the at least one weekly maintenance dose is orally administered.

55. The method of any one of claims 24 to 54, wherein the at least one weekly maintenance dose is administered in at least one tablet.

56. The method of any one of claims 24 to 54, wherein the at least one weekly maintenance dose is administered in an oral suspension.

57. The method of any one of claims 24 to 56, wherein the at least one weekly maintenance dose is administered without food.

58. The method of any one of claims 24 to 56, wherein the at least one weekly maintenance dose is administered with food.

59. The method of any one of claims 24 to 58, further comprising administering at least one additional active pharmaceutical ingredient.

60. The method of claim 59, wherein the at least one additional active pharmaceutical ingredient is chosen from antipsychotics.

61. The method of any one of claims 24 to 60, comprising treating at least one negative symptom of schizophrenia.

62. The method of claim 61, wherein the at least one negative symptom of schizophrenia is chosen from anhedonia, loss of motivation, and reduced interest in social interaction.

63. The method of any one of claims 24 to 62, comprising treating at least one cognitive symptom of schizophrenia.

64. The method of claim 63, wherein the at least one cognitive symptom of schizophrenia is chosen from impaired verbal memory, impaired working memory, impaired motor function, impaired attention and processing speed, impaired verbal fluency, and impaired executive function.

65. The method of any one of claims 24 to 64, comprising modulating reward anticipation related brain activity in the patient.

66. The method of any one of claims 24 to 65, comprising modulating cerebral blood flow in the patient.

67. The method of any one of claims 24 to 66, comprising increasing ventral striatal activity during reward anticipation in the patient.

68. The method of any one of claims 24 to 67, comprising modulating dopamine release in the patient.

69. A pharmaceutical composition comprising:
more than 80 mg of at least one compound chosen from Compound (I): 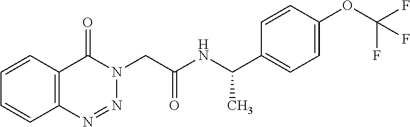 [SMILES: C[C@H](NC(=O)Cn1nnc2ccccc2c1=O)c1ccc(OC(F)(F)F)cc1]
and pharmaceutically acceptable salts thereof, and
at least one pharmaceutically acceptable excipient.

70. The pharmaceutical composition of claim 69, comprising more than 100 mg of at least one compound chosen from Compound (I) and pharmaceutically acceptable salts thereof.

71. The pharmaceutical composition of claim 69 or 70, comprising 120 mg of at least one compound chosen from Compound (I) and pharmaceutically acceptable salts thereof.

72. The pharmaceutical composition of claim 69 or 70, comprising 160 mg of at least one compound chosen from Compound (I) and pharmaceutically acceptable salts thereof.

73. The pharmaceutical composition of any one of claims 69 to 72, wherein the pharmaceutical composition is in the form of at least one tablet.

74. The pharmaceutical composition of any one of claims 69 to 72, wherein the pharmaceutical composition is in the form of an oral suspension.
